(19) United States
(12) Patent Application Publication
(10) Pub. No.: US 2017/0298443 A1
(43) Pub. Date: Oct. 19, 2017

(54) PROGNOSTIC TUMOR BIOMARKERS

(71) Applicant: MOFFITT GENETICS CORPORATION, Tampa, FL (US)

(72) Inventor: Hongyue A. Dai, Chesnut Hill, MA (US)

(21) Appl. No.: 15/514,147

(22) PCT Filed: Sep. 24, 2015
(86) PCT No.: PCT/US15/51868

§ 371 Date: Mar. 24, 2017

Related U.S. Application Data
(60) Provisional application No. 62/055,415, filed on Sep. 25, 2014.
(60) Provisional application No. 62/083,586, filed on Nov. 24, 2014.

(51) Int. Cl. C12Q 1/68

(52) U.S. Cl. CPC C12Q 1/6886; C12Q 2600/118; C12Q 2600/158

(57) ABSTRACT

Prognostic and predictive biomarkers are disclosed that can be used in systems and methods for predicting the prognosis of a subject with a cancer and to direct therapy based on that prognosis.

### CROSS-REFERENCE TO RELATED APPLICATIONS

This application claims benefit of U.S. Provisional Application No. 62/055,415, filed Sep. 25, 2014, and U.S. Provisional Application Ser. No. 62/083,586, filed Nov. 24, 2014, which are hereby incorporated herein by reference in their entirety.

### BACKGROUND

Cancer patients and their loved ones face many unknowns. Understanding their disease and what to expect can help patients and their loved ones make decisions about treatment, supportive and palliative care, rehabilitation, and personal matters, such as financial matters.

Many factors can influence the prognosis of a person with cancer. Among the most important are the type and location of the cancer, the stage of the disease (the extent to which the cancer has spread in the body), and the cancer's grade (how abnormal the cancer cells look under a microscope—an indicator of how quickly the cancer is likely to grow and spread). Other factors that affect prognosis include the biological and genetic properties of the cancer cells, the patient's age and overall general health, and the extent to which the patient's cancer responds to treatment.

Improved biomarkers and methods are needed to provide accurate and personalized prognosis for cancer patients.

### SUMMARY

Prognostic and predictive biomarkers are disclosed that were identified from gene expression profiling data from approximately 16,000 cancer subjects. These data were split into two parts. The first part, in combination with patient clinical data, was used to discover prognostic and predictive biomarkers for a series of different cancers capable and to train risk prediction models. These models were then validated using the second part of the gene expression profiling data. Therefore, systems and methods of using these biomarkers and predictive models are disclosed.

For example, a method for predicting prognosis of a patient with breast cancer is disclosed that involves the use of a composite model to predict the risk of bone metastasis and death. The method involves first determining gene expression intensities for several signature gene components from a tumor biopsy sample from the subject. In some embodiments, one of the components is estrogen receptor (ER) gene expression. In some embodiments, one of the components is human epidermal growth factor receptor 2 (HER2) gene expression. In some embodiments, one of the components is a proliferation signature gene score. This proliferation signature gene score can be generated using at least 1, 2, 3, 4, 5 6, 7, 8, 9, or 10 of the genes listed in Table 1, or genes highly correlated to the mean log expression of genes in Table 1, such as TPX2, CENPA, KIF2C, CCNB2, BUB1, HJURP, CDCA5, PTTG1, CEP55, and SKA1. In some embodiments, one of the components is an immune signature gene score. This immune signature gene score can be generated using at least 1, 2, 3, 4, 5 6, 7, 8, 9, or 10 of the genes listed in Table 2, or genes highly correlated to the mean log expression of genes in Table 2, such as CD3D, CD2, CD3E, ITK, TRBC1, TBC1D10C, ACAP1, CD247, SLAMF6, and IKZF1. The method can then involve calculating a breast cancer risk score from the gene expression intensities of each category, e.g., such that a high breast cancer risk score is an indication that the subject has a high risk for bone metastasis and/or death. The method can further involve treating the subject with more aggressive treatment if the subject has a high risk score. A more aggressive treatment for high score patients may include chemotherapy and bone metastasis preventive therapies like bisphosphonates, antibodies to RANKL or DKK1. For ER+ patients, more aggressive treatment for high score patients may include mTOR inhibitors, immune therapy like PD-1 inhibitors. For ER—patients, immune signature predicts relatively good outcome, so low-risk score in ER—maybe a selection factor for immune therapies like PD-1 or CTLA4 inhibitors. High risk patients could also be preferentially considered for genetic tests for targeted therapies like inhibitors for PI3K/AKT pathway. Patients with high immune signatures could be selected for immune therapies like anti-PD1. This prognostic model can be used to identify patients with unmet medical needs for new clinical trials for pharmaceutical companies, and to match case and control groups with similar prognostic levels for better clinical trial design for treatment efficacy.

Also disclosed is a method for predicting prognosis of a patient with lung cancer that also involves the use of a composite model to predict the risk of death. This method also involves first determining gene expression intensities for several signature gene components from a tumor biopsy sample from the subject. In some embodiments, one of the components is an immune signature gene score. This immune signature gene score can be generated using at least 1, 2, 3, 4, 5 6, 7, 8, 9, or 10 of the genes listed in Table 4, or genes highly correlated to the mean log expression of genes in Table 4, such as, CD2, ITGAL, IKZF1, CD3D, TRBC1, ACAP1, CD3E, TBC1D10C, CD247, and SLAMF6. In some embodiments, one of the components is a hypoxia signature gene score. This hypoxia signature gene score can be generated using at least 1, 2, 3, 4, 5 6, 7, 8, 9, or 10 of the genes listed in Table 5, or genes highly correlated to the mean log expression of genes in Table 5, such as SLC2A1, S100A2, KRT16, KRT6A, CD109, GJB3, SFN, MICALL1, RNTL2, and COL7A1. In some embodiments, one of the components is a lung cancer prognosis signature gene score. This lung cancer prognosis signature gene score can be generated using at least 1, 2, 3, 4, 5 6, 7, 8, 9, or 10 of the genes listed in Table 7, or genes highly correlated to the mean log expression of genes in Table 7, such as HLF, SCN7A, NR3C2, PCDP1, ABCA8, EMCN, IFT57, BDH2, MAMDC2, and ITGA8. In some embodiments, one of the components is a proliferation signature gene score. This proliferation score can be generated using at least 1, 2, 3, 4, 5 6, 7, 8, 9, or 10 of the genes listed in Table 8, or genes highly correlated to the mean log expression of genes in Table 8, such as TPX2, CENPA, KIF2C, CCNB2, CDCA5, HJURP, KIF4A, BIRC5, DLGAP5, and SKA1. The method can further involve determining the composite tumor stage. The method can then involve calculating a lung cancer risk score from the gene expression intensities of each category and the composite tumor stage, e.g., such that a high lung cancer risk score is an indication that the subject has a high risk for death. The method can further involve treating the subject with more aggressive treatment if the subject has a high risk score. For example, patients with high risk scores can be more aggressively treated with chemotherapies like cisplatin, carboplatin, docetaxel, or combinations. These patients could also be preferentially considered for genetic tests for targeted therapies like EGFR inhibitors or ALK inhibitors. Patients with high immune signatures could be selected for immune therapies like anti-PD1. This prognostic model can be used ti identify patients with unmet medical needs for new clinical trials for pharmaceutical companies, and to match case and control groups with similar prognostic levels for better clinical trial design for treatment efficacy.

Also disclosed is a method for predicting prognosis of a patient with colon cancer that also involves the use of a composite model to predict the risk of death. This method also involves first determining gene expression intensities for several signature gene components from a tumor biopsy sample from the subject. In some embodiments, one of the components is an immune signature gene score. This immune signature gene score can be generated using at least 1, 2, 3, 4, 5 6, 7, 8, 9, or 10 of the genes listed in Table 12, or genes highly correlated to the mean log expression of genes in Table 12, such as IKZF1, ITGAL, CD2, ITK, MAP4K1, CD3E, TBC1D10C, TRBC2, CD247, and CD3D. In some embodiments, one of the components is a hypoxia signature gene score. This hypoxia signature gene score can be generated using at least 1, 2, 3, 4, 5 6, 7, 8, 9, or 10 of the genes listed in Table 13, or genes highly correlated to the mean log expression of genes in Table 13, such as SLC2A1, RALA, ERO1L, ANLN, S100A2, PHLDA2, CDC20, LAMC2, PLAUR, and SLC16A3. In some embodiments, one of the components is a vimentin (VIM) correlated gene score. This VIM correlated gene score can be generated using at least 1, 2, 3, 4, 5 6, 7, 8, 9, or 10 of the genes listed in Table 14, or genes highly correlated to the mean log expression of genes in Table 14, such as CCDC80, VIM, HEG1, CNRIP1, RAB31, EFEMP2, GNB4, MRAS, CMTM3, and TIMP2. In some embodiments, one of the components is a CDH1 correlated gene score. This CDH1 correlated gene score can be generated using at least 1, 2, 3, 4, 5 6, 7, 8, 9, or 10 of the genes listed in Table 15, or genes highly correlated to the mean log expression of genes in Table 15, such as ELF3, CLDN7, CLDN4, CDH1, RAB25, ESRP1, ESRP2, ERBB3, AP1M2, and EPCAM. In some embodiments, one of the components is a first prognosis signature gene score. This first prognosis signature gene score can be generated using at least 1, 2, 3, 4, 5 6, 7, 8, 9, or 10 of the genes listed in Table 16, or genes highly correlated to the mean log expression of genes in Table 16, such as MZB1, OR6C4IGKV3-11 IGKV3D-11 IGKV3D-20 RHNO1, TNFRSF17, IGKC IGKV1D-39 IGKV1-39, IGHG1 IGH, IGLC1, IGKC IGKV1-16 IGKV1D-16, IGLV6-57, IGLV1-40 IGLV5-39, and IGJ. In some embodiments, one of the components is a second prognosis signature gene score. This second prognosis signature gene score can be generated using at least 1, 2, 3, 4, 5 6, 7, 8, 9, or 10 of the genes listed in Table 17, or genes highly correlated to the mean log expression of genes in Table 17, such as SPP1, CDH2, ITGB1, SERPINE1, PLOD2, COL4A1, NTM, MPRIP, PLIN2, and TIMP1. The method can further involve determining the composite tumor stage. The method can then involve calculating a colon cancer risk score from the gene expression intensities of each category and the composite tumor stage, e.g., such that a colon breast cancer risk score is an indication that the subject has a high risk of death. The method can further involve treating the subject with more aggressive treatment if the subject has a high risk score. For example, patients with high risk scores can be more aggressively treated with chemotherapies like 5_FU with leucovorin, or Camptosar and Eloxatin, or combinations. These patients could also be preferentially considered for genetic tests for targeted therapies like EGFR and VEGF inhibitors. Patients with high immune signatures could be selected for immune therapies like anti-PD1. This prognostic model can be used to identify patients with unmet medical needs for new clinical trials for pharmaceutical companies, and to match case and control groups with similar prognostic levels for better clinical trial design for treatment efficacy.

Also disclosed is a method for predicting prognosis of a patient with kidney cancer that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 22, or genes highly correlated to the mean log expression of genes in Table 22, such as CRY2, NR3C2, HLF, EMX2OS, FAM221B, BDH2, BCL2, ACADL, NDRG2, and NPR3. In some embodiments, one of the components is a second prognosis signature score. This second prognosis signature score can be generated using at least 1, 2, 3, 4, 5 6, 7, 8, 9, or 10 of the genes listed in Table 23, or genes highly correlated to the mean log expression of genes in Table 23, such as TPX2, CCNB2, AURKB, HJURP, CENPA, CENPF, SKA1, CEP55, PTTG1, and FOXM1. The method can then involve calculating a kidney cancer risk score from the gene expression intensities of each category, e.g., such that a high kidney cancer risk score is an indication that the subject has a high risk of death. The method can further involve treating the subject with more aggressive treatment if the subject has a high risk score. For example, patients with high risk scores can be more aggressively treated with immunotherapies and targeted with drugs like Sorafenib, Sunitinib, Tersirolimus, Everolimus, Avastin, Votrient, and Axitinib. This prognostic model can be used to identify patients with unmet medical needs for new clinical trials for pharmaceutical companies, and to match case and control groups with similar prognostic levels for better clinical trial design for treatment efficacy.

Also disclosed is a method for predicting prognosis of a patient with brain cancer that also involves the use of a composite model to predict the risk of death. This method also involves first determining gene expression intensities for several signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 26, or genes highly correlated to the mean log expression of genes in Table 26, such as HLF, CTBP2, CPEB3, SGMS1, CTBP2, ZRANB1, BTRC, ACADSB, ZC3H12B, and REPS2. In some embodiments, one of the components is a second prognosis signature score.

This second prognosis signature score can be generated using at least 1, 2, 3, 4, 5 6, 7, 8, 9, or 10 of the genes listed in Table 27, or genes highly correlated to the mean log expression of genes in Table 27, such as SKA1, TPX2, CCNB2, CENPA, BIRC5, RRM2, AURKA, AURKB, KIF2C, and CDCA8. In some embodiments, one of the components is a hypoxia signature score. This hypoxia signature score can be generated using at least 1, 2, 3, 4, 5 6, 7, 8, 9, or 10 of the genes listed in Table 28, or genes highly correlated to the mean log expression of genes in Table 28, such as TREM1, SERPINE1, HILPDA, RALA, AK2, SOD2, ARL4C, PGK1, ANGPTL4, and SLC16A3. The method can then involve calculating a brain cancer risk score from the gene expression intensities of each category, e.g., such that a high brain cancer risk score is an indication that the subject has a high risk of death. The method can further involve treating the subject with more aggressive treatment if the subject has a high risk score. For example, patients with high risk scores can be more aggressively treated with chemotherapies like cisplatin, carboplatin, methotrexate, or combinations. These patients could also be preferentially considered for genetic tests for targeted therapies like Avastin and Everolimus. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with prostate cancer that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 31, or genes highly correlated to the mean log expression of genes in Table 31, such as LMOD1, PGM5, MYLK, SYNPO2, SORBS1, PPP1R12B, DES, CNN1, MYH11, and MYOCD. In some embodiments, one of the components is a second prognosis signature score. This second prognosis signature score can be generated using at least 1, 2, 3, 4, 5 6, 7, 8, 9, or 10 of the genes listed in Table 32, or genes highly correlated to the mean log expression of genes in Table 32, such as TPX2, UBE2C, PTTG1, NUSAP1, CENPA, AURKA, CDCA5, NUSAP1, AURKB, and BIRC5. The method can then involve calculating a prostate cancer risk score from the gene expression intensities of each category, e.g., such that a high prostate cancer risk score is an indication that the subject has a high risk of death. The method can further involve treating the subject with more aggressive treatment if the subject has a high risk score. In general, prostate cancer patients have relatively good outcomes, so “watchful waiting” and hormonal therapies are common treatments for prostate cancer patients. However, patients with high risk scores have extremely poor outcome and should be treated aggressively by chemotherapies like docetaxel. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with pancreatic cancer that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 33, or genes highly correlated to the mean log expression of genes in Table 33, such as RUNDC3A, PCLO, SVOP, CELF4, CPLX2, SCG3, DNAJC6, AP3B2, SCN3B, and MPP2. In some embodiments, one of the components is a second prognosis signature score. This second prognosis signature score can be generated using at least 1, 2, 3, 4, 5 6, 7, 8, 9, or 10 of the genes listed in Table 33, or genes highly correlated to the mean log expression of genes in Table 33, such as SFN, LAMB3, TMPRSS4, PLEK2, MST1R, GJB3, S100A16, GPRC5A, PLAUR, and CAPG. The method can then involve calculating a pancreatic cancer risk score from the gene expression intensities of each category, e.g., such that a high pancreatic cancer risk score is an indication that the subject has a high risk of death. The method can further involve treating the subject with more aggressive treatment if the subject has a high risk score. In general, pancreatic cancer patients have very poor outcomes and should be treated aggressively. However, patients with low risk scores have good outcome and could be considered for less toxic treatments. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with endometrium cancer that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 35, or genes highly correlated to the mean log expression of genes in Table 35, such as PGR, UBXN10, SNTN, SPATA18, VWA3A, CDHR4, WDR96, STX18, ARMC3, and ESR1. In some embodiments, one of the components is a second prognosis signature score. This second prognosis signature score can be generated using at least 1, 2, 3, 4, 5 6, 7, 8, 9, or 10 of the genes listed in Table 36, or genes highly correlated to the mean log expression of genes in Table 36, such as MRGBP, UBE2S, GMPS, ACOT7, E2F1, CENPO, MRGBP, AURKA, BIRC5, and TPX2. The method can then involve calculating a endometrium cancer risk score from the gene expression intensities of each category, e.g., such that a high endometrium cancer risk score is an indication that the subject has a high risk of death. The method can further involve treating the subject with more aggressive treatment if the subject has a high risk score. In general, endometrium cancer patients have very poor outcomes and should be treated aggressively with chemo- and radiation-therapy. However, patients with low risk scores have good outcome and could be considered for less toxic treatments, like hormonal therapy. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with melanoma that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 37, or genes highly correlated to the mean log expression of genes in Table 37, such as IKZF3, CD3G, SH2D1A, SLAMF6, CD247, SLAMF6, SIRPG, TRAF3IP3, THEMIS, and TBC1D10C. In some embodiments, one of the components is a second prognosis signature score. This second prognosis signature score can be generated using at least 1, 2, 3, 4, 5 6, 7, 8, 9, or 10 of the genes listed in Table 38, or genes highly correlated to the mean log expression of genes in Table 38, such as ITFG3, TMEM201, TBC1D16, PPT2, GCAT, PAK4, OTUD7B, FITM2, PCGF2, and GCAT. The method can then involve calculating a melanoma risk score from the gene expression intensities of each category, e.g., such that a high melanoma risk score is an indication that the subject has a high risk of death. The method can further involve treating the subject with more aggressive treatment if the subject has a high risk score. In general, melanoma patients have very poor outcomes and should be treated aggressively. However, patients with low risk scores have good outcome and could be considered for less toxic treatments. This prognostic model can be used for identify patients with unmet medical needs for new clinical trials for pharmaceutical companies, and to match case and control groups with similar prognostic levels for better clinical trial design for treatment efficacy. One of the prognostic signatures is immune signature, and high immune signature score is correlated with good outcome, so the low risk score can also be used to select patients for immunotherapies like PD-1, PDL1 and CTLA4 antibodies. The melanoma prognosis model can also predict outcome of non-melanoma skin cancer patients.

Also disclosed is a method for predicting prognosis of a patient with soft tissue cancer that involves the use of correlated and anti-correlated biomarkers to predict the risk of death. This method involves first determining gene expression intensities for signature genes components from a tumor biopsy sample from the subject. In some embodiments, one of the components is a proliferation signature score. This proliferation signature score can be generated using at least 1, 2, 3, 4, 5 6, 7, 8, 9, or 10 of the genes listed in Table 44, or genes highly correlated to the mean log expression of genes in Table 44, such as TPX2, CCNB2, CENPA, SKA1, CCNB1, KIF2C, CDCA8, DEPDC1, CDCA5, BIRC5. In some embodiments, one of the components is a first prognosis signature score. This first prognosis signature score can be generated using at least 1, 2, 3, 4, 5 6, 7, 8, 9, or 10 of the genes listed in Table 40, or genes highly correlated to the mean log expression of genes in Table 40, such as EFCAB14, RGS5, EPS15, EFCAB14, IL33, SNRK, FBXL3, MBNL1, HIPK3, and CMAHP. In some embodiments, one of the components is a second prognosis signature score. This second prognosis signature score can be generated using at least 1, 2, 3, 4, 5 6, 7, 8, 9, or 10 of the genes listed in Table 41, or genes highly correlated to the mean log expression of genes in Table 41, such as MRPS12, ALYREF, SNRPB, LSM12, UBE2S, BANF1, LSM4, ANAPC11, HNRNPK, and RANBP1. The method can then involve calculating a soft tissue cancer risk score from the gene expression intensities of one or more of these components, e.g., such that a high soft tissue cancer risk score is an indication that the subject has a high risk of death. Treatment of soft tissue cancers includes surgery, radiation, chemo- and targeted therapies. The method can further involve treating the subject with more aggressive treatment if the subject has a high risk score. In general, soft tissue cancer patients have very poor outcomes and should be treated aggressively, including combinations of therapies. However, patients with low risk scores have good outcome and could be considered for less toxic treatments. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with uterine cancer that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 47, or genes highly correlated to the mean log expression of genes in Table 47, such as KIAA1324, CAPS, SCGB2A1, UBXN10, SOX17, RNF183, ASRGL1, UBXN10, SCGB1D2, and SPDEF. In some embodiments, one of the components is a second prognosis signature score. This second prognosis signature score can be generated using at least 1, 2, 3, 4, 5 6, 7, 8, 9, or 10 of the genes listed in Table 48, or genes highly correlated to the mean log expression of genes in Table 48, such as MRGBP, NUP155, GMPS, RYR1, FANCE, RFC4, UBE2S, ZNF623, ACOT7, and UCHL1. The method can then involve calculating a uterine cancer risk score from the gene expression intensities of each category, e.g., such that a high uterine cancer risk score is an indication that the subject has a high risk of death. The treatments to uterine cancer include surgery, radiation, hormonal (progestin) and chemotherapy. The method can further involve treating the subject with more aggressive treatment if the subject has a high risk score. In general, uterine cancer patients have very poor outcomes and should be treated aggressively, including combinations of therapies like hormonal+chemotherapies. However, patients with low risk scores have good outcome and could be considered for less toxic treatments like hormonal (progestin) only. Hormonal receptors like PGR and ESR1 are highly expressed in relative lower risk patients, making them a good target group for progestin treatment. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with ovarian cancer that involves stratification of patients using signature score by genes in Table 51, and then the use of correlated and anti-correlated biomarkers to predict the risk of death in the “signature-low” group. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 52, or genes highly correlated to the mean log expression of genes in Table 52, such as WDR96, DNAH6, TSNAXIP1, DNAH7, TTC18, PIFO, TTC25, NME5, WDR78, and DNAAF1. In some embodiments, one of the components is a second prognosis signature score. This second prognosis signature score can be generated using at least 1, 2, 3, 4, 5 6, 7, 8, 9, or 10 of the genes listed in Table 53, or genes highly correlated to the mean log expression of genes in Table 53, such as SPHK1, LINC00607, TNFAIP6, FAP, PTGIR, PLAU, TIMP3, INHBA, GPR68, and NTM. The method can then involve calculating an ovarian cancer risk score from the gene expression intensities of each category, e.g., such that a high ovarian cancer risk score is an indication that the subject has a high risk of death. The treatments for ovarian cancer include surgery and chemotherapy (platinum based and non-platinum based). The method can further involve treating the subject with more aggressive treatment if the subject has a high risk score. In general, ovarian cancer patients have very poor outcomes and should be treated aggressively. However, patients with low risk scores have good outcome and could be considered for less toxic treatments. This prognostic model can be used for identify patients with unmet medical needs for new clinical trials for pharmaceutical companies, and to match case and control groups with similar prognostic levels for better clinical trial design for treatment efficacy.

Also disclosed is a method for predicting prognosis of a patient with bladder cancer that involves the use of correlated and anti-correlated biomarkers to predict the risk of death. This method involves first determining gene expression intensities for two signature gene components from a tumor biopsy sample from the subject. In some embodiments, one of the components is a first prognosis signature score. This first prognosis signature score can be generated using at least 1, 2, 3, 4, 5 6, 7, 8, 9, or 10 of the genes listed in Table 57, or genes highly correlated to the mean log expression of genes in Table 57, such as ITGAL, IKZF1, CD3E, CD48, SLAMF6, CD2, TBC1D10C, PVRIG, CD5, and SLA2. In some embodiments, one of the components is a second prognosis signature score. This second prognosis signature score can be generated using at least 1, 2, 3, 4, 5 6, 7, 8, 9, or 10 of the genes listed in Table 58, or genes highly correlated to the mean log expression of genes in Table 58, such as KRT6B, DSC2, DSG3, FAM106B, KRT6A, KRT14, SPRR2D, RALA, SERPINB5, and RHCG. The method can then involve calculating bladder cancer risk score from the gene expression intensities of each category, e.g., such that a high bladder cancer risk score is an indication that the subject has a high risk of death. Treatment options for bladder cancer include surgery, radiation, chemo- and immune-therapies. The method can further involve treating the subject with more aggressive treatment if the subject has a high risk score. In general, bladder cancer patients have very poor outcomes and should be treated aggressively. However, patients with low risk scores have good outcome and could be considered for less toxic treatments, like immune therapies. One signature component is immune signature, and high immune signature is correlated with relatively good outcome. This suggests low-risk bladder patients are immune therapy target group. This prognostic model can be used for identify patients with unmet medical needs for new clinical trials for pharmaceutical companies, and to match case and control groups with similar prognostic levels for better clinical trial design for treatment efficacy.

In each of the above methods, risk scores can be calculate by any suitable computational predictive model, such as general linear regression, logistic regression, or simple linear/non-linear multivariate models with equal or unequal contributions from each component. In some case, the method involves simply summing the number of risk factors.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a graph showing that a 5-component model predicts average patient death rate in the validation set of primary breast cancer patients. X-axis: predicted death rate, Y-axis: actual average death rate, running average of 100 patients as ranked by the prediction.

FIG. 2 is a graph showing that the survival model predicts average bone metastasis rate in validation set of patients with primary tumor. X-axis: predicted death rate. Y-axis: average bone metastasis rate (running average of 100 samples ranked by predicted score).

FIG. 3 shows Kaplan-Meier plots for 1249 primary breast cancer patients in the validation set. Top curve: prediction score<0.15, Middle curve: score between 0.2 and 0.35, Bottom curve: score>0.35. The P-value for the Chi-square test is 0.

FIG. 4 is a graph showing that a 6-component model predicts average patient death rate in the validation set of lung cancer patients. X-axis: predicted death rate, Y-axis: actual average death rate, running average of 200 patients as ranked by the prediction.

FIG. 5 shows Kaplan-Meier plots for 1168 lung cancer patients in the validation set. Top curve: risk score<0.4, Middle curve: score between 0.4 and 0.7, Bottom curve: score>0.7. The P-value for the Chi-square test is 0.

FIG. 6 is a graph showing a 5-component model (based on reduced gene sets) predicts average patient death rate in the validation set of lung cancer patients. X-axis: predicted death rate, Y-axis: actual average death rate, running average of 200 patients as ranked by the prediction.

FIG. 7 shows Kaplan-Meier plots for 1168 lung cancer patients in the validation set (based on reduced gene sets). Top curve: risk score<0.4, Middle curve: score between 0.4 and 0.7, Bottom curve: score>0.7. The P-value for the Chi-square test is 0.

FIG. 8 is a graph showing microarray components (without tumor stage) predict average patient death rate in the validation set of lung cancer patients. X-axis: predicted death rate, Y-axis: actual average death rate, running average of 200 patients as ranked by the prediction.

FIG. 9 is a graph showing an 8-component model predicts average patient death rate in the validation set of colon cancer patients. X-axis: predicted death rate, Y-axis: actual average death rate, running average of 200 patients as ranked by the prediction.

FIG. 10 shows Kaplan-Meier plots for 1057 colon cancer patients in the validation set. Top curve: risk score<0.2, Middle curve: score between 0.2 and 0.5, Bottom curve: score>0.5. The P-value for the Chi-square test is 3.86×10⁻¹².

FIG. 11 is a graph showing a 7-component model predicts average patient death rate in colon cancer patients (based on reduced gene sets). X-axis: predicted death rate, Y-axis: actual average death rate, running average of 200 patients as ranked by the prediction.

FIG. 12 shows Kaplan-Meier plots for 1057 colon cancer patients in the validation set (based on reduced gene sets). Top curve: risk score<0.25, Middle curve: score between 0.25 and 0.5, Bottom curve: score>0.5. The P-value for the Chi-square test is 3.7×10⁻¹³.

FIG. 13 is a graph showing microarray components (without tumor stage) predict average patient death rate in colon cancer patients. X-axis: predicted death rate, Y-axis: actual average death rate, running average of 200 patients as ranked by the prediction.

FIG. 14 is a graph showing a 2-component model predicts average patient death rate in validation set of kidney cancer patients. X-axis: predicted death rate, Y-axis: actual average death rate, running average of 100 patients as ranked by the prediction.

FIG. 15 shows Kaplan-Meier plots for 444 kidney cancer patients in the validation set. Top curve: risk score<0.35, Middle curve: score between 0.35 and 0.6, Bottom curve: score>0.6. The P-value for the Chi-square test is 2.4×10⁻¹⁴. Note the K-M curves are biased given significant number of follow-up dates are missing for the good outcome patients. The chi-square test p-value is still correct since it only uses live/death information in each group).

FIG. 16 is a graph showing a 2-component model predicts average patient death rate in kidney cancer patients (based on reduced gene sets). X-axis: predicted death rate, Y-axis: actual average death rate, running average of 100 patients as ranked by the prediction.

FIG. 17 shows Kaplan-Meier plots for 444 kidney cancer patients in the validation set (based on reduced gene sets). Top curve: risk score<0.35, Middle curve: score between 0.35 and 0.6, Bottom curve: score>0.6. The P-value for the Chi-square test is 1.4×10⁻¹⁵. Note the K-M curves are biased given significant number of follow-up dates are missing for the good outcome patients. The chi-square test p-value is still correct since it only uses live/death information in each group).

FIG. 18 is a graph showing a 3-component model predicts average patient death rate in the validation set of brain cancer patients. X-axis: predicted death rate, Y-axis: actual average death rate, running average of 100 patients as ranked by the prediction.

FIG. 19 shows Kaplan-Meier plots for 257 brain cancer patients in the validation set. Top curve: risk score<0.4, Middle curve: score between 0.4 and 0.75, Bottom curve: score>0.75. The P-value for the Chi-square test is 3.2×10⁻¹³. Note the K-M curves are biased given significant number of follow-up dates are missing for the good outcome patients. The chi-square test p-value is still correct since it only uses live/death information in each group)

FIG. 20 is a graph showing a 3-component model predicts average patient death rate in brain cancer patients (based on reduced gene sets). X-axis: predicted death rate, Y-axis: actual average death rate, running average of 100 patients as ranked by the prediction.

FIG. 21 shows Kaplan-Meier plots for 257 brain cancer patients in the validation set (based on reduced gene sets). Top curve: risk score<0.4, Middle curve: score between 0.4 and 0.75,

Bottom curve: score>0.75. The P-value for the Chi-square test is 6.8×10⁻¹³. Note the K-M curves are biased given significant number of follow-up dates are missing for the good outcome patients. The chi-square test p-value is still correct since it only uses live/death information in each group).

FIG. 22 is a Kaplan-Meier plots for 151 prostate cancer patients in the validation set. Top curve: risk score<0.4, Bottom curve: score>0.4. The P-value for the Chi-square test is 0. Note the K-M curves are biased given significant number of follow-up dates are missing for the good outcome patients. The chi-square test p-value is still correct since it only uses live/death information in each group).

FIG. 23 is a Kaplan-Meier plots for 151 prostate cancer patients in the validation set (based on reduced gene sets). Top curve: risk score<0.4, Bottom curve: score>0.4. The P-value for the Chi-square test is 0. Note the K-M curves are biased given significant number of follow-up dates are missing for the good outcome patients. The chi-square test p-value is still correct since it only uses live/death information in each group).

FIG. 24 shows Kaplan-Meier plots for 263 pancreatic cancer patients in the validation set. Top curve: risk score<0.5, Bottom curve: score>0.5. The P-value for the Chi-square test is 5.82×10⁻⁹. Note the K-M curves are biased given significant number of follow-up dates are missing for the good outcome patients. The chi-square test p-value is still correct since it only uses live/death information in each group).

FIG. 25 shows Kaplan-Meier plots for 263 pancreatic cancer patients in the validation set (based on reduced gene sets). Top curve: risk score<0.5, Bottom curve: score>0.5. The P-value for the Chi-square test is 3.8×10⁻⁸. Note the K-M curves are biased given significant number of follow-up dates are missing for the good outcome patients. The chi-square test p-value is still correct since it only uses live/death information in each group.

FIG. 26 is a plot showing a 3-component model predicts average patient death rate in the validation set of endometrium cancer patients. X-axis: predicted death rate, Y-axis: actual average death rate, running average of 50 patients as ranked by the prediction.

FIG. 27 shows Kaplan-Meier plots for 184 endometrium cancer patients in the validation set (based on reduced gene sets). Top curve: risk score<0.2, Middle curve: score between 0.2 and 0.4, Bottom curve: score>0.4. The P-value for the Chi-square test is 9.7×10⁻⁵.

FIG. 28 shows Kaplan-Meier plots for 184 endometrium cancer patients in the validation set. Top curve: risk score<0.2, Middle curve: score between 0.2 and 0.4, Bottom curve: score >0.4. The P-value for the Chi-square test is 1.0×10⁻⁴.

FIG. 29 is a plot showing a 2-component model predicts average patient death rate in the validation set melanoma patients. X-axis: predicted death rate, Y-axis: actual average death rate, running average of 50 patients as ranked by the prediction.

FIG. 30 shows Kaplan-Meier plots for 153 melanoma patients in the validation set. Top curve: risk score<0.45, Middle curve: score between 0.45 and 0.65, Bottom curve: score>0.65. The P-value for the Chi-square test is 9.3×10⁻⁹.

FIG. 31 is a plot showing a 2-component model predicts average patient death rate in melanoma patients (based on reduced gene sets). X-axis: predicted death rate, Y-axis: actual average death rate, running average of 50 patients as ranked by the prediction.

FIG. 32 shows Kaplan-Meier plots for 153 melanoma patients in the validation set (based on reduced gene sets). Top curve: risk score<0.45, Middle curve: score between 0.45 and 0.6, Bottom curve: score>0.6. The P-value for the Chi-square test is 1.0×10⁻⁷.

FIG. 33 shows Kaplan-Meier plots for 152 other skin cancer patients excluding malignant melanoma. Top curve: risk score<0.45, Middle curve: score between 0.45 and 0.6, Bottom curve: score>0.6. The P-value for the Chi-square test is 9.2×10⁻⁴.

FIG. 34 is a graph showing a 2-component model predicts average patient death rate in the validation set of soft tissue cancer patients. X-axis: predicted death rate, Y-axis: actual average death rate, running average of 50 patients as ranked by the prediction.

FIG. 35 shows Kaplan-Meier plots for 95 soft tissue cancer patients in the validation set. Top curve: risk score<0.34, Middle curve: score between 0.34 and 0.55, Bottom curve: score >0.55. The P-value for the Chi-square test is 1.1×10⁻⁴. Note the K-M curves are biased given significant number of follow-up dates are missing for the good outcome patients. The chi-square test p-value is still correct since it only uses live/death information in each group).

FIG. 36 shows Kaplan-Meier plots for 95 soft tissue cancer patients in the validation set (based on reduced gene sets). Top curve: risk score<0.34, Middle curve: score between 0.34 and 0.55, Bottom curve: score>0.55. The P-value for the Chi-square test is 3.2×10⁻⁴. Note the K-M curves are biased given significant number of follow-up dates are missing for the good outcome patients. The chi-square test p-value is still correct since it only uses live/death information in each group).

FIG. 37 is a plot showing model based on proliferation signature predicts average patient death rate in soft tissue cancer patients. X-axis: predicted death rate, Y-axis: actual average death rate, running average of 50 patients as ranked by the prediction.

FIG. 38 shows Kaplan-Meier plots based on proliferation signature for 95 soft tissue cancer patients in the validation set. Top curve: risk score<0.42, Middle curve: score between 0.42 and 0.55, Bottom curve: score>0.55. The P-value for the Chi-square test is 2.3×10⁻⁴. Note the K-M curves are biased given significant number of follow-up dates are missing for the good outcome patients. The chi-square test p-value is still correct since it only uses live/death information in each group).

FIG. 39 shows Kaplan-Meier plots for 95 soft tissue cancer patients in the validation set (based on reduced proliferation geneset). Top curve: risk score<0.4, Middle curve: score between 0.4 and 0.55, Bottom curve: score>0.55. The P-value for the Chi-square test is 1.2×10⁻⁴. Note the K-M curves are biased given significant number of follow-up dates are missing for the good outcome patients. The chi-square test p-value is still correct since it only uses live/death information in each group).

FIG. 40 shows Kaplan-Meier plots for 95 soft tissue cancer patients in the validation set, by the average risk score. Top curve: risk score<0.4, Middle curve: score between 0.4 and 0.55, Bottom curve: score>0.55. The P-value for the Chi-square test is 1.2×10⁻⁴. Note the K-M curves are biased given significant number of follow-up dates are missing for the good outcome patients. The chi-square test p-value is still correct since it only uses live/death information in each group).

FIG. 41 shows Kaplan-Meier plots for 95 soft tissue cancer patients in the validation set, by the number of risk factors (RF). Top curve: RF =0, Middle RF =1, Bottom curve: RF =2. The P-value for the Chi-square test is 5.7×10⁻⁵. Note the K-M curves are biased given significant number of follow-up dates are missing for the good outcome patients. The chi-square test p-value is still correct since it only uses live/death information in each group).

FIG. 42 is a plot showing a 3-component model predicts average patient death rate in the validation set of uterus cancer patients. X-axis: predicted death rate, Y-axis: actual average death rate, running average of 50 patients as ranked by the prediction.

FIG. 43 shows Kaplan-Meier plots for 153 uterus cancer patients in the validation set. Top curve: risk score<0.32, Middle curve: score between 0.32 and 0.6, Bottom curve: score>0.6. The P-value for the Chi-square test is 2.1×10⁻⁹.

FIG. 44 is a plot showing a 3-component model predicts average patient death rate in uterus cancer patients (based on reduced gene sets). X-axis: predicted death rate, Y-axis: actual average death rate, running average of 50 patients as ranked by the prediction.

FIG. 45 shows Kaplan-Meier plots for 153 uterus cancer patients in the validation set (based on reduced gene sets). Top curve: risk score<0.32, Middle curve: score between 0.32 and 0.6, Bottom curve: score>0.6. The P-value for the Chi-square test is 1.3×10⁻⁹.

FIG. 46 is a histogram of X2 intensities (average of log2 intensities from all probes in Table 51).

FIG. 47 is a plot showing estrogen-receptor (ER) intensity vs. X2 intensity. High-X2 patients have uniform high ER levels.

FIG. 48 is a plot showing a 3-component model predicts average patient death rate in X2-ovarian cancer patients. X-axis: predicted death rate, Y-axis: actual average death rate, running average of 50 patients as ranked by the prediction.

FIG. 49 shows Kaplan-Meier plots for 170 X2-ovarian cancer patients in the validation set. Top curve: risk score<0.5, Middle curve: score between 0.5 and 0.7, Bottom curve: score >0.7. The P-value for the Chi-square test is 3.6×10⁻⁷. Note the K-M curves are biased given significant number of follow-up dates are missing for the good outcome patients. The chi-square test p-value is still correct since it only uses live/death information in each group.

FIGS. 50A and 50B show Kaplan-Meier plots for signatures (FIG. 50A) and tumor stage (FIG. 50B) in 170 X2-ovarian cancer patients of the validation set. In FIG. 50A, Top curve: risk score<0, Middle curve: score between 0 and 0.2, Bottom curve: score>0.2. The Chi-square for 2 degree of freedom is 34. In FIG. 50B, Top curve: tumor stage 0, 1, 2; Middle curve: tumor stage 3; Bottom curve: tumor stage 4. The Chi-square for 2 degree of freedom is 27.9.

FIG. 51 is a plot showing a 3-component model predicts average patient death rate in X2-ovarian cancer patients (based on reduced gene sets). X-axis: predicted death rate, Y-axis: actual average death rate, running average of 50 patients as ranked by the prediction.

FIG. 52 shows Kaplan-Meier plots for 170 X2-ovarian cancer patients in the validation set. Top curve: risk score<0.5, Middle curve: score between 0.5 and 0.7, Bottom curve: score >0.7. The P-value for the Chi-square test is 2.1×10⁻⁷. Note the K-M curves are biased given significant number of follow-up dates are missing for the good outcome patients. The chi-square test p-value is still correct since it only uses live/death information in each group.

FIGS. 53A and 53B are histograms of immune signature score for X2-(FIG. 53A) and X2+ (FIG. 53B) patients.

FIG. 54 shows the correlation between CDH6 and X2 (correlation=0.61).

FIGS. 55A and 55B are Kaplan-Meier curves for X2-population (FIG. 55A) and X2+ population (FIG. 55B).

FIG. 56 shows Kaplan-Meier plots for 136 bladder cancer patients in the validation set. Top curve: risk score<0.66, Middle curve: score between 0.66 and 0.75, Bottom curve: score >0.75. The P-value for the Chi-square test is 1.3×10⁻³. Note the K-M curves are biased given significant number of follow-up dates are missing for the good outcome patients. The chi-square test p-value is still correct since it only uses live/death information in each group.

FIG. 57 shows Kaplan-Meier plots for 136 bladder cancer patients in the validation set (based on reduced gene sets). Top curve: risk score<0.5, Middle curve: score between 0.5 and 0.75, Bottom curve: score>0.75. The P-value for the Chi-square test is 2.2×10⁻³. Note the K-M curves are biased given significant number of follow-up dates are missing for the good outcome patients. The chi-square test p-value is still correct since it only uses live/death information in each group.

### DETAILED DESCRIPTION

Prognostic and predictive biomarkers are disclosed that can be used in systems and methods for predicting the prognosis of a cancer patient, which can be used to guide therapeutic and palliative treatment of the patient. The methods generally involve determining gene expression of a panel of biomarkers and use of these gene expression intensities calculate predictive risk scores.

Gene Expression Assays

Methods of “determining gene expression levels” include methods that quantify levels of gene transcripts as well as methods that determine whether a gene of interest is expressed at all. A measured expression level may be expressed as any quantitative value, for example, a fold-change in expression, up or down, relative to a control gene or relative to the same gene in another sample, or a log ratio of expression, or any visual representation thereof, such as, for example, a “heat crap” where a color intensity is representative of the amount of gene expression detected. Exemplary methods for detecting the level of expression of a gene include, but are not limited to, Northern blotting, dot or slot blots, reporter gene matrix, nuclease protection, RT-PCR, microarray profiling, differential display, 2D gel electrophoresis, SELDI-TOF, ICAT, enzyme assay, antibody assay, and MNAzyme-based detection methods. Optionally a gene whose level of expression is to be detected may be amplified, for example by methods that may include one or more of: polymerase chain reaction (PCR), strand displacement amplification (SDA), loop-mediated isothermal amplification (LAMP), rolling circle amplification (RCA), transcription-mediated amplification (TMA), self-sustained sequence replication (3SR), nucleic acid sequence based amplification (NASBA), or reverse transcription polymerase chain reaction (RT- PCR).

A number of suitable high throughput formats exist for evaluating expression patterns and profiles of the disclosed genes. Numerous technological platforms for performing high throughput expression analysis are known. Generally, such methods involve a logical or physical array of either the subject samples, the biomarkers, or both. Common array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., xMAP® technology from Luminex (Austin, Tex.), the SECTOR® Imager with MULTI-ARRAY® and MULTI-SPOT® technologies from Meso Scale Discovery (Gaithersburg, Md.), the ORCA™ system from Beckman-Coulter, Inc. (Fullerton, Calif.) and the ZYMATETM systems from Zymark Corporation (Hopkinton, Mass.), miRCURY LNA™ microRNA Arrays (Exiqon, Woburn, Mass.).

Alternatively, a variety of solid phase arrays can favorably be employed to determine expression patterns in the context of the disclosed methods, assays and kits. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

Microarray expression may be detected by scanning the microarray with a variety of laser or CCD-based scanners, and extracting features with numerous software packages, for example, IMAGENE™ (Biodiscovery), Feature Extraction Software (Agilent), SCANLYZE™ (Stanford Univ., Stanford, Calif.), GENEPIX™ (Axon Instruments).

In some cases, single molecule sequencing methods are used determining gene expression patterns. In some embodiments, amplified cDNA is sequenced by whole transcriptome shotgun sequencing (also referred to herein as (“RNA-Seq”). Whole transcriptome shotgun sequencing (RNA-Seq) can be accomplished using a variety of next-generation sequencing platforms such as the Illumina Genome Analyzer platform, ABI Solid Sequencing platform, or Life Science's 454 Sequencing platform.

In some embodiments, the nCounter® Analysis system (Nanostring Technologies, Seattle, Wash.) is used to detect intrinsic gene expression. This system is described in International Patent Application Publication No. WO 08/124,847 and U.S. Pat. No. 8,415,102, which are each incorporated herein by reference in their entireties for the teaching of this system. The basis of the nCounter® Analysis system is the unique code assigned to each nucleic acid target to be assayed.

The code is composed of an ordered series of colored fluorescent spots which create a unique barcode for each target to be assayed. A pair of probes is designed for each DNA or RNA target, a biotinylated capture probe and a reporter probe carrying the fluorescent barcode. This system is also referred to, herein, as the nanoreporter code system.

Specific reporter and capture probes can be synthesized for each target. Briefly, sequence-specific DNA oligonucleotide probes are attached to code-specific reporter molecules. Preferably, each sequence specific reporter probe comprises a target specific sequence capable of hybridizing to no more than one target and optionally comprises at least two, at least three, or at least four label attachment regions, said attachment regions comprising one or more label monomers that emit light. Capture probes are made by ligating a second sequence-specific DNA oligonucleotide for each target to a universal oligonucleotide containing biotin. Reporter and capture probes are all pooled into a single hybridization mixture, the “probe library”.

The relative abundance of each target is measured in a single multiplexed hybridization reaction. The method comprises contacting a biological sample with a probe library, the library comprising a probe pair for gene target, such that the presence of the target in the sample creates a probe pair target complex. The complex is then purified. More specifically, the sample is combined with the probe library, and hybridization occurs in solution. After hybridization, the tripartite hybridized complexes (probe pairs and target) are purified in a two-step procedure using magnetic beads linked to oligonucleotides complementary to universal sequences present on the capture and reporter probes, This dual purification process allows the hybridization reaction to be driven to completion with a large excess of target-specific probes, as they are ultimately removed, and, thus, do not interfere with binding and imaging of the sample. All post hybridization steps are handled robotically on a custom liquid-handling robot (Prep Station, NanoString Technologies).

Purified reactions are deposited by the Prep Station into individual flow cells of a sample cartridge, bound to a streptavidin-coated surface via the capture probe, electrophoresed to elongate the reporter probes, and immobilized. After processing, the sample cartridge is transferred to a fully automated imaging and data collection device (Digital Analyzer, NanoString Technologies). The expression level of a target is measured by imaging each sample and counting the number of times the code for that target is detected. Data is output in simple spreadsheet format listing the number of counts per target, per sample.

This system can be used along with nanoreporters. Additional disclosure regarding nanoreporters can be found in International Publication No. WO 07/076,129 and WO 07/076,132, and US Patent Publication No. 2010/0015607 and 2010/0261026, the contents of which are incorporated herein in their entireties. Further, the term nucleic acid probes and nanoreporters can include the rationally designed (e.g, synthetic sequences) described in International Publication No. WO 2010/019826 and US Patent Publication No. 2010/0047924, incorporated herein by reference in its entirety.

Calculation of Risk Score

From the disclosed gene expression values, a dataset can be generated and inputted into an analytical classification process that uses the data to classify the biological sample with a risk score. The data may be obtained via any technique that results in an individual receiving data associated with a sample. For example, an individual may obtain the dataset by generating the dataset himself by methods known to those in the art. Alternatively, the dataset may be obtained by receiving a dataset or one or more data values from another individual or entity. For example, a laboratory professional may generate certain data values while another individual, such as a medical professional, may input all or part of the dataset into an analytic process to generate the result.

Prior to input into the analytical process, the data in each dataset can be collected by measuring the values for each biomarker gene, usually in duplicate or triplicate or in multiple replicates. The data may be manipulated, for example raw data may be transformed using standard curves, and the average of replicate measurements used to calculate the average and standard deviation for each patient. These values may be transformed before being used in the models.

For example, it is often useful to pre-process gene expression data, for example, by addressing missing data, translation, scaling, normalization, weighting, etc. Multivariate projection methods, such as principal component analysis (PCA) and partial least squares analysis (PLS), are so-called scaling sensitive methods. By using prior knowledge and experience about the type of data studied, the quality of the data prior to multivariate modeling can be enhanced by scaling and/or weighting. Adequate scaling and/or weighting can reveal important and interesting variation hidden within the data, and therefore make subsequent multivariate modeling more efficient. Scaling and weighting may be used to place the data in the correct metric, based on knowledge and experience of the studied system, and therefore reveal patterns already inherently present in the data.

If possible, missing data, for example gaps in column values, should be avoided. However, if necessary, such missing data may replaced or “filled” with, for example, the mean value of a column (“mean fill”); a random value (“random fill”); or a value based on a principal component analysis (“principal component fill”). In some cases, there are multiple genes from the same pathway signature, and the missing data of a particular genes can be modeled by correlated genes in the same pathway.

“Translation” of the descriptor coordinate axes can be useful. Examples of such translation include normalization and mean centering. “Normalization” may be used to remove sample-to-sample variation. Some commonly used methods for calculating normalization factor include: (i) global normalization that uses all genes on the array; (ii) housekeeping genes normalization that uses constantly expressed housekeeping/invariant genes; and (iii) internal controls normalization that uses known amount of exogenous control genes added during hybridization. In some embodiments, the intrinsic genes disclosed herein can be normalized to control housekeeping genes. It will be understood by one of skill in the art that the methods disclosed herein are not bound by normalization to any particular housekeeping genes, and that any suitable housekeeping gene(s) known in the art can be used.

Many normalization approaches are possible, and they can often be applied at any of several points in the analysis. In one embodiment, data is normalized using the LOWESS method, which is a global locally weighted scatter plot smoothing normalization function. In another embodiment, data is normalized to the geometric mean of set of multiple housekeeping genes.

“Mean centering” may also be used to simplify interpretation. Usually, for each descriptor, the average value of that descriptor for all samples is subtracted. In this way, the mean of a descriptor coincides with the origin, and all descriptors are “centered” at zero. In “unit variance scaling,” data can be scaled to equal variance. Usually, the value of each descriptor is scaled by 1/StDev, where StDev is the standard deviation for that descriptor for all samples. “Pareto scaling” is, in some sense, intermediate between mean centering and unit variance scaling. In pareto scaling, the value of each descriptor is scaled by 1/sqrt(StDev), where StDev is the standard deviation for that descriptor for all samples. In this way, each descriptor has a variance numerically equal to its initial standard deviation. The pareto scaling may be performed, for example, on raw data or mean centered data.

“Logarithmic scaling” may be used to assist interpretation when data have a positive skew and/or when data spans a large range, e.g., several orders of magnitude. Usually, for each descriptor, the value is replaced by the logarithm of that value. In “equal range scaling,” each descriptor is divided by the range of that descriptor for all samples. In this way, all descriptors have the same range, that is, 1. However, this method is sensitive to presence of outlier points. In “autoscaling,” each data vector is mean centered and unit variance scaled. This technique is a very useful because each descriptor is then weighted equally, and large and small values are treated with equal emphasis. This can be important for genes expressed at very low, but still detectable, levels.

Data can also be normalized by the method described by Welsh et al. BMC Bioinformatics. 2013 14:153, which is incorporated by reference for its teaching of these algorithms and methods.

The methods described herein may be implemented and/or the results recorded using any device capable of implementing the methods and/or recording the results. Examples of devices that may be used include but are not limited to electronic computational devices, including computers of all types. When the methods described herein are implemented and/or recorded in a computer, the computer program that may be used to configure the computer to carry out the steps of the methods may be contained in any computer readable medium capable of containing the computer program. Examples of computer readable medium that may be used include but are not limited to diskettes, CD-ROMs, DVDs, ROM, RAM, and other memory and computer storage devices. The computer program that may be used to configure the computer to carry out the steps of the methods and/or record the results may also be provided over an electronic network, for example, over the internet, an intranet, or other network.

This data can then be input into the analytical process with defined parameter, The analytic classification process may be any type of learning algorithm with defined parameters, or in other words, a predictive model. In general, the analytical process will be in the form of a model generated by a statistical analytical method such as those described below. Examples of such analytical processes may include a linear algorithm, a quadratic algorithm, a polynomial algorithm, a decision tree algorithm, or a voting algorithm.

Using any suitable learning algorithm, an appropriate reference or training dataset can be used to determine the parameters of the analytical process to be used for classification, i.e., develop a predictive model. The reference or training dataset ⁻to be used will depend on the desired classification to be determined, The dataset may include data from two, three, four or more classes,

The number of features that may be used by an analytical process to classify a test subject with adequate certainty is 2 or more, in some embodiments, it is 3 or more, 4 or more, 10 or more, or between 10 and 74. Depending on the degree of certainty sought, however, the number of features used in an analytical process can be more or less, but in all cases is at least 2. In one embodiment, the number of features that may be used by an analytical process to classify a test subject is optimized to allow a classification of a test subject with high certainty.

Suitable data analysis algorithms are known in the art. In one embodiment, a data analysis algorithm of the disclosure comprises Classification and Regression Tree (CART), Multiple Additive Regression Tree (MART), Prediction Analysis for Microarrays (PAM), or Random Forest analysis. Such algorithms classify complex spectra from biological materials to distinguish subjects as normal or as possessing biomarker levels characteristic of a particular disease state. In other embodiments, a data analysis algorithm of the disclosure comprises ANOVA and nonparametric equivalents, linear discriminant analysis, logistic regression analysis, nearest neighbor classifier analysis, neural networks, principal component analysis, quadratic discriminant analysis, regression classifiers and support vector machines. While such algorithms may be used to construct an analytical process and/or increase the speed and efficiency of the application of the analytical process and to avoid investigator bias, one of ordinary skill in the art will realize that computer-based algorithms are not required to carry out the methods of the present disclosure.

As will be appreciated by those of skill in the art, a number of quantitative criteria can be used to communicate the performance of the comparisons made between a test marker profile and reference marker profiles. These include area under the curve (AUC), hazard ratio (HR), relative risk (RR), reclassification, positive predictive value (PPV), negative predictive value (NPV), accuracy, sensitivity and specificity, Net reclassification Index, Clinical Net reclassification Index. In addition, other constructs such a receiver operator curves (ROC) can be used to evaluate analytical process performance.

Predicting Cancer Survivability

The disclosed biomarkers, systems, methods, assays, and kits can be used to predict the survivability of a subject with a cancer. The disclosed biomarkers, methods, assays, and kits are particularly useful to predict the benefit of aggressive treatment. For example, the cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

Adjuvant Therapy

The calculated risk scores can be used to predict the benefit of an adjuvant therapy for a subject based on their expected survivability. In some embodiments, the method also predicts the efficacy of adjuvant therapy in the subject. Adjuvant therapy is additional treatment given after surgery to reduce the risk that the cancer will come back. Adjuvant treatment may include chemotherapy (the use of drugs to kill cancer cells) and/or radiation therapy (the use of high energy x-rays to kill cancer cells).

The disclosed risk scores can be used to identify whether the subject will have improve survivability if treated with adjuvant chemotherapy (ACT) and may also predict benefit of radiation therapy. For example, the method can involve administering ACT and/or radiation therapy to the subject if a high risk score is calculated.

Definitions

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “prognosis” refers to a predicted clinical outcome that can be used by a clinician to select an appropriate treatment. This term includes estimations of survival, tumor progression (e.g., metastasis), and/or responsiveness to treatment.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A number of embodiments of the invention have been described. Nevertheless, it will be understood that various modifications may be made without departing from the spirit and scope of the invention. Accordingly, other embodiments are within the scope of the following claims.

### EXAMPLES

Gene expression profiling data was generated for approximately 16,000 cancer subjects. This dataset is the biggest and one of the best quality dataset in the world. It was generated using a uniform protocol (NuGen) on a uniform platform (Merck version of Affymetrix® arrays).

The gene expression data in combination with patient clinical follow-up data (overall survival, response to standard care treatments, etc.) was used to discover prognostic or predictive biomarkers. There are more than 10 tumor types or subtypes with adequate number of samples to derive the prognosis signatures. For example, there are nearly 4,000 breast cancer samples, 500 brain tumors, 880 kidney tumors, 3,000 lung tumors and more than 2,000 colon tumors in the profiling dataset.

For those tumor types or subtypes with adequate number of samples, the approach for biomarker discovery was to divide the samples equally into two parts: the first half samples used for biomarker discovery and model training, and the second half used for validation.

Within the training samples, a modified method based on a previous publication (Dai H, et al. Cancer Res. 2005 65(10):4059-66) was used to discover two groups of biomarkers (correlated and anti-correlated to the survival). The mean log expression level of each biomarker group in each sample was computed, and the mean log expression of each group, or the difference of the mean log expression between these two groups of biomarkers was used to build a survival prediction model in the training samples. The same model was then applied to the reserved validation samples to estimate the performance.

For tumor-types with more than one or two mechanisms involved in affecting the final outcome, a composite model was developed to include these factors. For example, the factors can be pathway scores, single gene markers, or histo-pathological parameters.

### Example 1

### Prognostic Model for Breast Cancer

Proliferation is a strong predictor of metastasis or death in ER+breast cancer patients. Studies also linked estrogen receptor (ER) level and Her2 level to breast cancer patient outcome. In addition, it was observed in the dataset that the immune signature is related to good outcome in breast cancer patient, especially in ER-patients. For a strong predictor, all these factors can be included.

A composite model was therefore built in 2,000 breast cancer training samples. The model contained ER and HER2 expression levels as measured by array probes, average proliferation score measured by 100 proliferation genes, and immune score measured by 100 immune related genes.

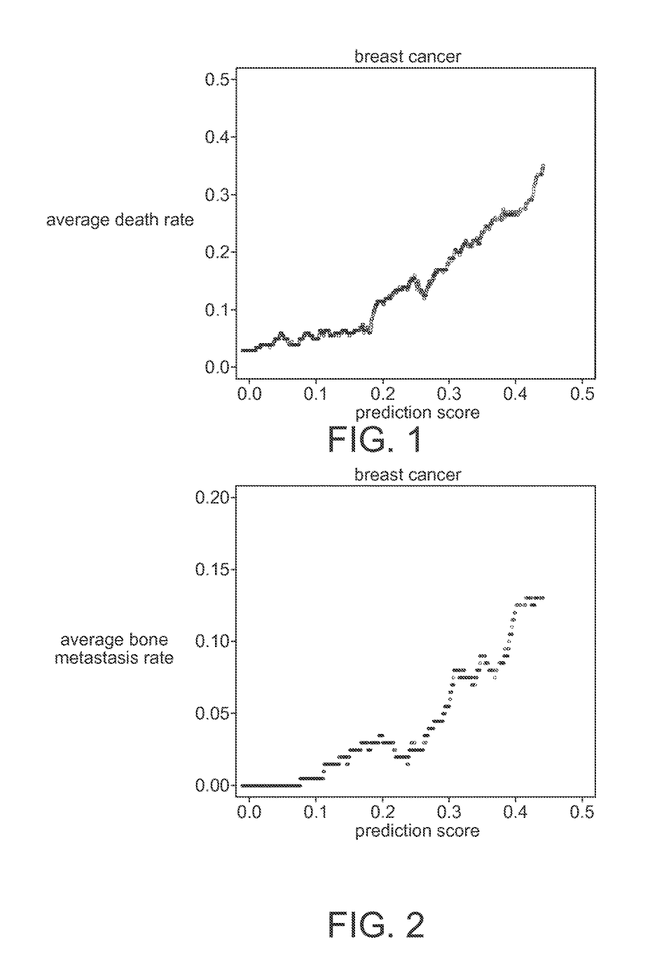

The performance of this model was evaluated in reserved validation set of 2,000 samples. The validation set contains 1249 unique primary patients and 166 unique metastatic patients, with some samples profiled multiple times. FIG. 1 shows the predicted death rate vs. the actual average (running average of 100 samples as ranked by the prediction score) death rate in unique primaries. As shown in the Figure, the model predicts the average death rate very well.

The odds ratio in all 1,249 validation primary patients is 5.99, 95% CI [4.00, 8.98]. The predictor is independently predictive in each well define clinical sub-populations. In ER+patients, the odds ratio was 5.4, 95% CI [3.3, 8.9]. In ER-patients, the odds ratio was 4.8, 95% CI [2.2, 10.3]. In the metastatic population, the odds ratio was 8.4, 95% CI [3.1, 22.6].

This same model also predicts the bone metastasis in primary breast cancer patients. FIG. 2 shows the actual average bone metastasis rate vs. the predicted death rate. A strong correlation is observed between these two rates. Among 672 patients with low predicted score, 6 developed metastasis (0.9%), whereas in the 577 patients with high predicted score, 41 developed bone metastasis (7.1%), Fisher's exact test P-value is 4.2×10⁻⁹.

Based on the predictive score by the model, patients can be further divided into good (score <0.2), medium (0.2<score<0.35) and poor (score>0.35) prognosis groups. The actual death rates from the primary validation sets were 4.8% (32/672), 16.6% (62/373) and 34.8% (71/204).

In the validation set, there were 637 primary patients with lymph node negative (LNO) and 496 primary patients with lymph node positive (LN1, 2, 3) breast cancer. When the model was applied to the LN− and LN+positive groups, the odds ratios for the overall survival were 5.78, 95% CI[3.12, 10.69], and 5.06, 95% CI[2.54, 10.07] respectively. For the bone metastasis, in the LN−, the total bone metastasis rat is 1% (7/637), hence the prediction is not significant. In the LN+ group, the bone metastasis rates were 0.0% (0/179) and 9.8% (31/317), P-value=7.4×10⁻⁷.

When patients were divided up into age groups (less than 55 years and great than 55 years), the overall survival odds ratios were 9.15, 95% CI[3.57, 23.44], and 5.96, 95% CI[3.75, 9.45] respectively. The bone metastasis rates in the younger patient group were 1.9% (4/208) vs. 8.8% (23/261) for the low and high risk score groups (P=0.001). For the older patient group, the rates were 0.4% (2/464) vs. 5.7% (18/316), P-value=4.8×10⁻⁸.

When patients were divided into tumor grade groups 1&2, and 3, the overall survival odds ratios were 6.18 95% CI[3.78, 10.12] and 6.11, 95% CI[2.86, 13.07], respectively. In grade 1&2 patients, the bone metastasis rates were 0.4% (2/491) vs. 7.8% (22/282) for the low and high risk groups, P-value=1.6×10⁻⁸. For grade 3 patients, the rates were 2.2% (4/181) vs. 6.4% (19/295), P-value=0.05.

Materials & Methods

The 5 components used to determine a breast cancer risk score were: ER, measured by gene expression probe targeting NM_000125, in log2 scale; HER2, measured by gene expression probe, targeting NM_03_2339, in log2 scale; proliferation signature score, measured by mean log2 intensities of the genes in Table 1; immune signature score, measured by mean log2 intensities of the genes in Table 2; and composite stage based on histology and clinical stage.

The formulas used for calculating the breast prediction score were:

Breast Cancer Risk Score=0.653031+(−0.027485*ER)+(0.004901*HER2)+(0.047574*Proliferation)+(−0.071552*immune) (Formula 1a),

where a high score means high risk.

Breast Cancer Risk Score=0.546072+(−0.025403*ER)+(−0.004187*HER2)+(0.042013*Proliferation)+(−0.073342*immune)+(0.126162*stage) (Formula 1b),

where a high score means high risk.

TABLE 1
100 Proliferation genes

| Probe | Gene |
|---|---|
| merck-CR596700_a_at | RRM2 |
| merck2-AL517462_s_at | — |
| merck-NM_145060_at | SKA1 |
| merck-NM_198436_s_at | AURKA |
| merck2-NM_001039535_a_at | SKA1 |
| merck2-NM_145060_a_at | SKA1 |
| merck-ENST00000333706_x_at | BIRC5 |
| merck-AK223428_a_at | BIRC5 |
| merck-NM_004219_x_at | PTTG1 |
| merck-NM_012310_at | KIF4A GDPD2 |
| merck-NM_001809_at | CENPA |
| merck2-ENST00000333706_s_at | — |
| merck-NM_001276_at | CHI3L1 |
| merck-NM_018101_at | CDCA8 |
| merck-ENST00000360566_at | RRM2 |
| merck2-BC001651_at | CDCA8 |
| merck2-AF098158_at | TPX2 |
| merck-NM_012112_at | TPX2 |
| merck-NM_005733_at | KIF20A CDC23 |
| merck-U63743_a_at | KIF2C |
| merck2-AK123247_at | MYH11 NDE1 |
| merck2-ENST00000331944_s_at | — |
| merck-NM_181802_at | UBE2C |
| merck2-NM_018410_at | HJURP |
| merck2-BT006759_at | KIF2C |
| merck2-M87338_at | RFC2 |
| merck-NM_152637_at | METTL7B ITGA7 |
| merck-NM_182513_at | SPC24 |
| merck-NM_018154_at | ASF1B PRKACA |
| merck2-AL519719_a_at | BIRC5 |
| merck2-BC007417_at | POC1A |
| merck-NM_021953_at | FOXM1 |
| merck-NM_016426_at | GTSE1 TRMU |
| merck-CR602926_s_at | CCNB1 |
| merck-NM_014791_at | MELK |
| merck-NM_006342_at | TACC3 |
| merck-NM_004701_at | CCNB2 |
| merck-NM_004217_at | AURKB |
| merck-NM_144569_s_at | SPOCD1 |
| merck2-NM_001168_at | BIRC5 |
| merck2-BC006325_at | GTSE1 TRMU |
| merck-NM_018131_at | CEP55 |
| merck-AY605064_at | CLSPN |
| merck-NM_004336_at | BUB1 RGPD6 |
| merck-NM_031299_at | CDCA3 GNB3 |
| merck2-AF043294_at | BUB1 RGPD6 |
| merck2-NM_014397_at | NEK6 |
| merck-NM_001255_s_at | CDC20 |
| merck2-ENST00000370966_a_at | DEPDC1 OTUD7A |
| merck-ENST00000243201_a_at | HJURP |
| merck-NM_003258_at | TK1 |
| merck-CR602847_a_at | KIAA0101 |
| merck-NM_006547_at | IGF2BP3 AMOTL1 MALSU1 |
| merck2-BC006325_x_at | GTSE1 TRMU |
| merck-BC075828_a_at | GTSE1 |
| merck-NM_014750_at | DLGAP5 |
| merck-NM_203394_at | E2F7 |
| merck-ENST00000308604_s_at | LINC00152 MIR4435-1HG |
| merck-AF469667_a_at | MLF1IP |
| merck-BI868409_a_at | MKI67 |
| merck-NM_016639_at | TNFRSF12A CLDN9 |
| merck-CR607300_a_at | MKI67 |
| merck-NM_001237_a_at | CCNA2 EXOSC9 |
| merck-NM_152515_at | CKAP2L |
| merck-AK055931_a_at | SHCBP1 |
| merck-NM_005192_at | CDKN3 |
| merck2-AK000490_a_at | DEPDC1 |
| merck-NM_012291_at | ESPL1 PFDN5 |
| merck-BC106033_s_at | SMC4 |
| merck2-BC034607_at | ASPM |
| merck-NM_152562_s_at | CDCA2 |
| merck-NM_004237_at | TRIP13 |
| merck2-AK026140_at | — |
| merck-NM_001813_at | CENPE |
| merck2-BC005978_at | KPNA2 |
| merck2-NM_024745_at | SHCBP1 |
| merck-CR610123_a_at | POC1A |
| merck-NM_001790_at | CDC25C |
| merck2-Y00472_a_at | SOD2 |
| merck2-BC025232_at | CDC6 |
| merck2-NM_017779_at | DEPDC1 |
| merck-NM_004526_at | MCM2 |
| merck2-BC107750_at | CDK1 RHOBTB1 |
| merck-BX649059_at | GAS2L3 |
| merck-NM_005480_at | TROAP |
| merck-NM_007243_a_at | NRM |
| merck2-NM_031966_at | CCNB1 |
| merck-NM_001024466_s_at | SOD2 |
| merck2-BC005978_s_at | KPNA2 |
| merck-NM_080668_at | CDCA5 |
| merck-NM_004911_at | PDIA4 |
| merck-BC004202_a_at | CHEK1 |
| merck-NM_003504_at | CDC45 |
| merck2-BC098582_at | KIF14 |
| merck2-M36693_a_at | SOD2 |
| merck-NM_012145_a_at | DTYMK |
| merck-NM_017581_at | CHRNA9 |
| merck2-BM464374_at | CENPE |
| merck-NM_001845_at | COL4A1 |
| merck2-DQ890621_at | CDC45 |

TABLE 2
100 immune signature genes

| probe | Gene |
|---|---|
| merck-NM_003151_a_at | STAT4 |
| merck2-AJ515553_at | AMICA1 |
| merck-NM_153206_s_at | AMICA1 |
| merck-NM_006682_s_at | FGL2 CCDC146 |
| merck-NM_000733_at | CD3E |
| merck-BC030533_s_at | TRBC1 TRBV19 |
| merck-NM_001767_at | CD2 |
| merck-BC014239_s_at | PTPRC |
| merck-NM_001040067_s_at | TRBC2 TRBV3-1 |
| | TRBV5-4 TRBV6-5 TRBV7-2 |
| merck-NM_002209_at | ITGAL |
| merck-NM_080612_at | GAB3 |
| merck2-ENST00000390420_at | TRBV3-1 TRBV5-4 TRBV6-5 |
| | TRBV7-2 |
| merck2-AA669142_at | — |
| merck-NM_002104_at | GZMK |
| merck-NM_005546_at | ITK CYFIP2 |
| merck-NM_018384_at | GIMAP5 GIMAP1-GIMAP5 |
| merck2-ENST00000390409_at | TRBC1 TRBV19 |
| merck-NM_153236_at | GIMAP7 |
| merck2-ENST00000390420_s_at | — |
| merck2-ENST00000390537_s_at | — |
| merck-NM_003650_at | CST7 |
| merck-NM_001504_at | CXCR3 |
| merck-NM_000732_at | CD3D |
| merck-AI281804_at | GPR174 |
| merck-ENST00000382913_s_at | TRAC TRAJ17 TRAV20 TRDV2 |
| merck2-NM_198196_a_at | CD96 |
| merck-NM_001558_at | IL10RA |
| merck-NM_002832_at | PTPN7 |
| merck-NM_005335_at | HCLS1 |
| merck2-NM_001558_at | IL10RA |
| merck2-AL833681_at | CD96 |
| merck-NM_175900_s_at | C16orf54 QPRT |
| merck-AK021632_at | ANKRD44 |
| merck2-NM_175900_at | C16orf54 QPRT |
| merck-NM_003978_at | PSTPIP1 |
| merck-NM_032214_at | SLA2 |
| merck-NM_014207_at | CD5 |
| merck2-NM_005816_a_at | CD96 |
| merck2-NM_001114380_x_at | ITGAL |
| merck2-DB317311_at | GIMAP1 |
| merck-NM_001781_at | CD69 |
| merck-NM_030767_at | AKNA |
| merck-ENST00000318430_s_at | TMC8 |
| merck2-AW798052_at | AKNA |
| merck2-NM_002209_x_at | ITGAL |
| merck-NM_016388_at | TRAT1 |
| merck-NM_002298_s_at | LCP1 |
| merck-NM_007360_at | KLRK1 KLRC4-KLRK1 |
| merck-NM_024070_at | PVRIG |
| merck-NM_005816_at | CD96 |
| merck2-BM977026_at | — |
| merck-NM_017424_at | CECR1 |
| merck-NM_032496_at | ARHGAP9 |
| merck-NM_130848_s_at | C5orf20 |
| merck2-NM_177405_a_at | CECR1 |
| merck-NM_001037631_at | CTLA4 ICOS |
| merck2-NM_145642_a_at | APOL3 |
| merck-BC017813_a_at | FGL2 CCDC146 |
| merck-AK025758_at | NFATC2 |
| merck2-NM_014349_a_at | APOL3 |
| merck2-NM_145640_a_at | APOL3 |
| merck-BE856897_s_at | NFATC2 |
| merck2-NM_030644_a_at | APOL3 |
| merck2-NM_145639_a_at | APOL3 |
| merck-ENST00000381961_at | IL7R |
| merck2-AA278761_at | — |
| merck-NM_014716_at | ACAP1 |
| merck-NM_000206_at | IL2RG |
| merck2-NM_007360_at | KLRK1 KLRC4-KLRK1 |
| merck-ENST00000343625_s_at | RASAL3 |
| merck-BG271748_s_at | GIMAP1 |
| merck-NM_000734_at | CD247 |
| merck-NM_003387_at | WIPF1 |
| merck-NM_005541_at | INPP5D |
| merck2-NM_145641_a_at | APOL3 |
| merck-BX648371_at | LINC00861 |
| merck2-NM_017424_a_at | CECR1 |
| merck-NM_001838_at | CCR7 |
| merck-CR617832_a_at | MS4A1 |
| merck2-BX640915_at | TIGIT |
| merck-NM_006725_at | CD6 |
| merck-NM_198517_at | TBC1D10C |
| merck-BC028068_s_at | JAK3 INSL3 |
| merck2-NM_006120_at | HLA-DMA BRD2 |
| merck-NM_001079_at | ZAP70 |
| merck-AF402776_at | MIR155HG |
| merck-NM_014879_at | P2RY14 |
| merck-NM_052931_at | SLAMF6 |
| merck-NM_022141_at | PARVG |
| merck-NM_018460_at | ARHGAP15 |
| merck-NM_001025265_at | CXorf65 |
| merck-NM_024898_s_at | DENND1C CRB3 |
| merck-NM_001001895_at | UBASH3A |
| merck-ENST00000316577_s_at | TESPA1 |
| merck2-BC020657_at | GIMAP4 |
| merck-NM_004877_at | GMFG |
| merck-M21624_s_at | TRDC |
| merck2-BM678246_at | CD37 |
| merck-NM_018556_s_at | SIRPG |
| merck-NM_145641_s_at | APOL3 |

The number of genes in each pathway was reduced to 10 genes.

Proliferation:
Probe IDs: merck-NM_012112_at, merck-NM_001809 at merck-U63743_a_at, merck-NM_004701_at, merck2-AF043294_at, merck-ENST00000243201_a_at, merck-NM_080668_at, merck-NM_004219_x_at merck-NM_018131_at merck-NM_145060_at
Gene symbols: TPX2, CENPA, KIF2C, CCNB2, BUB1, HJURP, CDCA5, PTTG1, CEP55, SKA1

Immune Signature:
Probe IDs: merck-NM_000732_at, merck-NM_001767_at, merck-NM_000733_at, merck-NM_005546_at, merck2-ENST00000390409_at, merck-NM_198517_at, merck-NM_014716_at, merck-NM_000734_at, merck-NM_052931_at, merck2-B1519527_at
Gene symbols: CD3D, CD2, CD3E, ITK, TRBC1, TBC1D10C, ACAP1, CD247, SLAMF6, IKZF1

The scores derived from these 10-genes correlated to the original scores at the level of 0.99 for both proliferation and immune score. The formula for calculating the prediction score is:

Breast Cancer Risk Score=0.404457 (−0.026432*ER)+(−0.001974*HER2)+(0.034656*Proliferation)+(−0.054045*immune)+(0.127414*stage) (Formula 2).

This model predicts breast cancer patient outcome (overall survival) in 1249 primary breast cancer validation set. For example, at the threshold of 0.2, the odds ratio is 5.31 (95% CI: 3.57-7.88). The Fisher's Exact Test P-value is 9.8×10⁻²⁰.

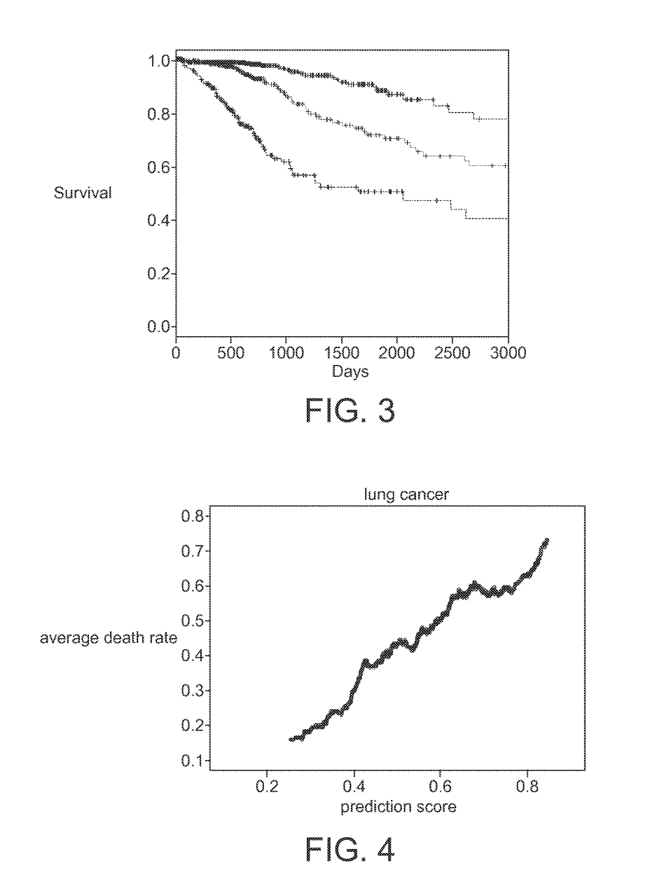

The validation patients can be further divided into good, medium and poor prognosis groups. FIG. 3 shows the Kaplan-Meier curves for patients with prediction score<0.2 (good prognosis), 0.2-0.35 (medium prognosis) and >0.35 (poor prognosis) respectively. The P-value based on Chi-square test is 0.

The risk of death increases linearly with the prediction score. Table 3 illustrates the death rate and bone metastasis rate vs. prediction scores.

TABLE 3
Death rate and bone metastasis rate verses prediction score

| Prediction | Number of | Number of | | | Bone Mets |
|---|---|---|---|---|---|
| score | samples | deaths | Death rate | Bone mets | rate |
| <0 | 110 | 1 | 0.009 | 0 | 0.000 |
| 0-0.1 | 252 | 12 | 0.048 | 0 | 0.000 |
| 0.1-0.2 | 300 | 21 | 0.070 | 7 | 0.023 |
| 0.2-0.3 | 278 | 40 | 0.144 | 7 | 0.025 |
| 0.3-0.4 | 166 | 36 | 0.217 | 14 | 0.084 |
| >0.4 | 143 | 55 | 0.385 | 19 | 0.133 |

### Example 2

### Prognostic Model for Lung Cancer

This example describes a lung cancer prognosis model which uses gene expression profiling data and tumor stage. The model contains multiple gene expression signatures as components and the tumor stage. In the second part of the example, the number of genes in each signature is reduced to 10 genes to simplify the implementation of this prognosis model.

There are numerous studies of prognoses using gene expression alone, or histopathology/clinical data alone. Here we combine both to further improve the prognosis.

A total of 2,978 samples were profiled by Affymetrix® expression arrays. A composite model was built using the first half of samples and the model validated using the second half of samples. In the first half of samples, 1,456 samples had outcome data (live or death), and 1,339 patients had tumor stage measurement. In the second half of samples, 1,486 had outcome data, and 1,168 patients had stage measurement.

The model was built in the training set using a general linear model (from the R package) using the following equation:

Lung Cancer Risk Score=−0.54238+(−0.04826*imscore)+(0.04317*hscore)+(0.03468*ras)+(−0.01188*prg)+(0.09167*pscore)+(0.07474*stage) (Formula 3),

where “imscore” is an immune score calculated from immune signature genes in Table 4, “hscore” is a hypoxia score from hypoxia signature genes in Table 5, “ras” is a score from ras signature genes in Table 6, “prg” is a score calculated from prognosis genes listed in Table 7, “pscore” is a proliferation score from the proliferation signature genes in Table 8, and the stage is the composite tumor stage. Scores for each signature was computed simply by averaging the log2 expression level of the genes in the signature.

TABLE 4
Immune signature genes

| probe | Gene |
|---|---|
| merck-NM_005356_at | LCK |
| merck-NM_006144_at | GZMA |
| merck-NM_014207_at | CD5 |
| merck-NM_005608_at | PTPRCAP |
| merck-NM_007181_at | MAP4K1 |
| merck-NM_002738_at | PRKCB |
| merck-Y00638_s_at | PTPRC |
| merck-BC014239_s_at | PTPRC |
| merck-NM_130446_at | KLHL6 |
| merck-NM_005546_at | ITK CYFIP2 |
| merck-NM_006257_at | PRKCQ |
| merck-NM_002104_at | GZMK |
| merck-NM_001504_at | CXCR3 |
| merck-NM_001001895_at | UBASH3A |
| merck-NM_002832_at | PTPN7 |
| merck-NM_018460_at | ARHGAP15 |
| merck-NM_001838_at | CCR7 |
| merck-NM_002209_at | ITGAL |
| merck-NM_006725_at | CD6 |
| merck-BC028068_s_at | JAK3 INSL3 |
| merck-NM_001079_at | ZAP70 |
| merck-NM_005541_at | INPP5D |
| merck-ENST00000318430_s_at | TMC8 |
| merck-NM_006564_at | CXCR6 |
| merck-NM_007237_s_at | SP140 |
| merck-NM_178129_at | P2RY8 |
| merck-NM_000647_s_at | CCR2 |
| merck-BU428565_s_at | P2RY8 |
| merck-NM_002351_s_at | SH2D1A |
| merck-NM_001040033_at | CD53 |
| merck-NM_005816_at | CD96 |
| merck-NM_198517_at | TBC1D10C |
| merck-NM_000733_at | CD3E |
| merck-NM_002163_at | IRF8 |
| merck-NM_000655_at | SELL |
| merck-NM_003037_at | SLAMF1 |
| merck-NM_003151_a_at | STAT4 |
| merck-NM_001007231_s_at | ARHGAP25 |
| merck-NM_018326_at | GIMAP4 |
| merck-NM_000377_at | WAS |
| merck-NM_001558_at | IL10RA |
| merck-NM_002985_at | CCL5 |
| merck-DT807100_at | CD3D CD3G |
| merck-NM_001465_at | FYB |
| merck-BP339517_a_at | FYB |
| merck-NM_030767_at | AKNA |
| merck-NM_005565_at | LCP2 |
| merck-NM_001040031_at | CD37 |
| merck-NM_002872_at | RAC2 |
| merck-NM_019604_at | CRTAM |
| merck-NM_005263_at | GFI1 |
| merck-NM_001037631_at | CTLA4 ICOS |
| merck-NM_016388_at | TRAT1 |
| merck-NM_014450_at | SIT1 RMRP |
| merck-NM_000732_at | CD3D |
| merck-NM_000073_at | CD3G |
| merck-NM_007360_at | KLRK1 KLRC4-KLRK1 |
| merck-NM_013351_at | TBX21 |
| merck-NM_032214_at | SLA2 |
| merck-NM_000639_at | FASLG |
| merck-NM_001242_at | CD27 |
| merck-ENST00000381961_at | IL7R |
| merck-NM_153206_s_at | AMICA1 |
| merck-NM_001025598_at | ARHGAP30 USF1 |
| merck-NM_001768_at | CD8A |
| merck-NM_003978_at | PSTPIP1 |
| merck-NM_014716_at | ACAP1 |
| merck-AK128740_s_at | IL16 |
| merck-NM_006060_a_at | IKZF1 |
| merck-BC075820_at | IKZF1 |
| merck-NM_016293_at | BIN2 |
| merck-NM_012092_at | ICOS |
| merck-NM_005442_at | EOMES LOC100996624 |
| merck-NM_007074_at | CORO1A |
| merck-NM_000206_at | IL2RG |
| merck-NM_005041_at | PRF1 |
| merck-NM_024898_s_at | DENND1C CRB3 |
| merck-NM_173799_at | TIGIT |
| merck-NM_001767_at | CD2 |
| merck-NM_002348_at | LY9 |
| merck-X60502_s_at | SPN QPRT |
| merck-NM_153236_at | GIMAP7 |
| merck-NM_005601_at | NKG7 |
| merck-NM_032496_at | ARHGAP9 |
| merck-NM_004877_at | GMFG |
| merck-NM_021181_at | SLAMF7 |
| merck-NM_018384_at | GIMAP5 GIMAP1-GIMAP5 |
| merck-NM_181780_at | BTLA |
| merck-NM_001017373_at | SAMD3 |
| merck-NM_000734_at | CD247 |
| merck-NM_003650_at | CST7 |
| merck-NM_172101_at | CD8B |
| merck-NM_001803_at | CD52 |
| merck-NM_001778_at | CD48 |
| merck-NM_001025265_at | CXorf65 |
| merck-NM_198929_at | PYHIN1 |
| merck-ENST00000379833_at | GVINP1 |
| merck-NM_052931_at | SLAMF6 |
| merck-NM_001024667_s_at | FCRL3 |
| merck-NM_002258_at | KLRB1 |
| merck-NM_018556_s_at | SIRPG |
| merck-AK090431_s_at | NLRC3 |
| merck-NM_018990_at | SASH3 XPNPEP2 |
| merck-NM_175900_s_at | C16orf54 QPRT |
| merck-ENST00000316577_s_at | TESPA1 |
| merck-NM_024070_at | PVRIG |
| merck-AY190088_s_at | — |
| merck-NM_001040067_s_at | TRBC2 TRBV3-1 TRBV5-4 |
| | TRBV6-5 TRBV7-2 |
| merck-NM_130848_s_at | C5orf20 |
| merck-ENST00000381153_at | C11orf21 |
| merck-ENST00000382913_s_at | TRAC TRAJ17 TRAV20 TRDV2 |
| merck-BC030533_s_at | TRBC1 TRBV19 |
| merck-ENST00000244032_a_at | ZNF831 |
| merck-ENST00000371030_at | ZNF831 |
| merck-ENST00000343625_s_at | RASAL3 |
| merck-AF143887_at | — |
| merck-AK128436_at | IKZF3 |
| merck-AI281804_at | GPR174 |
| merck-AF086367_at | — |
| merck-CR598049_at | LINC00426 |
| merck-BM700951_at | KLRK1 KLRC4-KLRK1 |
| merck-BX648371_at | LINC00861 |
| merck-BC070382_at | — |
| merck2-AW798052_at | AKNA |
| merck2-BX640915_at | TIGIT |
| merck2-BM678246_at | CD37 |
| merck2-NM_025228_at | TRAF3IP3 |
| merck2-XM_033379_at | WDFY4 |
| merck2-AJ515553_at | AMICA1 |
| merck2-BP262340_at | IL16 |
| merck2-AK225623_at | DENND1C CRB3 |
| merck2-AL833681_at | CD96 |
| merck2-BF111803_at | ARHGAP15 |
| merck2-BX406128_at | CD3G |
| merck2-NM_153701_at | — |
| merck2-BC020657_at | GIMAP4 |
| merck2-AY185344_at | PYHIN1 |
| merck2-DR159064_at | EOMES LOC100996624 |
| merck2-ENST00000390420_at | TRBV3-1 TRBV5-4 TRBV6-5 |
| | TRBV7-2 |
| merck2-ENST00000390420_s_at | — |
| merck2-NM_001010923_at | THEMIS |
| merck2-ENST00000390409_at | TRBC1 TRBV19 |
| merck2-AX721088_at | — |
| merck2-ENST00000390393_at | TRBV19 |
| merck2-AW341086_at | — |
| merck2-AA278761_at | — |
| merck2-AA278761_x_at | — |
| merck2-ENST00000390394_s_at | — |
| merck2-AA669142_at | — |
| merck2-AW007991_at | PTPRC |
| merck2-BG743900_at | PRKCB |
| merck2-X06318_at | PRKCB |
| merck2-BI519527_at | IKZF1 |
| merck2-ENST00000390537_s_at | — |
| merck2-AY292266_x_at | — |
| merck2-NM_005816_a_at | CD96 |
| merck2-NM_198196_a_at | CD96 |
| merck2-NM_001114380_x_at | ITGAL |
| merck2-NM_007237_a_at | SP140 |
| merck2-NM_007237_at | SP140 |
| merck2-NM_052931_at | SLAMF6 |
| merck2-NM_001558_at | IL10RA |
| merck2-NM_007360_at | KLRK1 KLRC4-KLRK1 |
| merck2-NM_002209_x_at | ITGAL |
| merck2-NM_175900_at | C16orf54 QPRT |

TABLE 5
Hypoxia signature genes

| probe | Gene |
|---|---|
| merck-NM_002627_at | PFKP PITRM1 |
| merck-NM_000302_at | PLOD1 |
| merck-NM_001216_at | CA9 RMRP |
| merck-ENST00000377093_at | KIF1B |
| merck-BC004202_a_at | CHEK1 |
| merck-NM_030949_at | PPP1R14C |
| merck-CR593119_a_at | CLIC4 |
| merck-NM_001255_s_at | CDC20 |
| merck-BG679113_s_at | KRT6A KRT6B KRT6C |
| merck-NM_002421_at | MMP1 |
| merck-BQ217236_a_at | SERPINB5 |
| merck-NM_001793_at | CDH3 |
| merck-NM_001238_at | CCNE1 |
| merck-BU597348_s_at | SYNCRIP |
| merck-NM_006516_at | SLC2A1 |
| merck-BX648425_a_at | DSC2 |
| merck-X15014_a_at | RALA |
| merck-NM_018685_at | ANLN |
| merck-CR614206_a_at | ERO1L |
| merck-NM_001124_at | ADM |
| merck-NM_015440_at | MTHFD1L |
| merck-ENST00000367307_a_at | MTHFD1L |
| merck-NM_058179_at | PSAT1 |
| merck-NM_031415_s_at | GSDMC |
| merck-NM_005557_x_at | KRT16 |
| merck-NM_053016_at | PALM2 PALM2-AKAP2 |
| merck-CR602579_a_at | CTPS1 |
| merck-NM_001428_s_at | ENO1 |
| merck-ENST00000305850_at | CENPN CMC2 |
| merck-NM_005978_at | S100A2 |
| merck-NM_018643_at | TREM1 |
| merck-NM_006505_at | PVR |
| merck-NM_080655_s_at | MSANTD3 |
| merck-NM_001012507_at | CENPW |
| merck-ENST00000258005_a_at | NHSL1 |
| merck-AK129763_at | LINC00673 |
| merck-XM_927868_s_at | PGK1 |
| merck-XM_928117_x_at | FAM106B |
| merck-AL359337_at | ADM |
| merck-AA148856_s_at | SYNCRIP |
| merck2-AI989728_at | SERPINB5 |
| merck2-DQ892208_at | CA9 RMRP |
| merck2-AK022036_at | WWTR1 |
| merck2-AA677426_at | — |
| merck2-AA677426_s_at | — |
| merck2-BC004856_at | NCS1 |
| merck2-BG252150_at | PFKP |
| merck2-BC007633_at | AGO2 |
| merck2-BG400371_at | — |
| merck2-DQ891441_at | — |
| merck2-NM_017522_AS_at | LRP8 |
| merck2-AF039652_at | RNASEH1 |
| merck2-AV714642_at | ANLN |
| merck2-AB030656_at | CORO1C |
| merck2-NM_000291_at | PGK1 |
| merck2-NM_005554_at | KRT6A |
| merck2-BC002829_at | S100A2 |
| merck2-BU681245_at | — |
| merck2-AK225899_a_at | CTPS1 |
| merck2-BC062635_a_at | XPO5 |
| merck2-AF257659_a_at | CALU |
| merck2-CA308717_at | — |
| merck2-X56807_at | DSC2 |
| merck2-CR936650_at | ANLN |
| merck2-AY423725_a_at | PGK1 |
| merck2-BC103752_a_at | PGK1 |

TABLE 6
Ras signature genes

| probe | Gene |
|---|---|
| merck-NM_002205_at | ITGA5 |
| merck-NM_000376_at | VDR |
| merck-NM_002203_at | ITGA2 |
| merck-NM_002658_at | PLAU |
| merck-CD014069_s_at | TNFRSF1A |
| merck-NM_004419_at | DUSP5 |
| merck-NM_021199_s_at | SQRDL |
| merck-NM_016639_at | TNFRSF12A CLDN9 |
| merck-NM_002068_at | GNA15 |
| merck-NM_005562_at | LAMC2 |
| merck-BG677853_a_at | LAMC2 |
| merck-BM980789_s_at | LAMC2 |
| merck-ENST00000265539_s_at | FOSL2 |
| merck-NM_013451_at | MYOF |
| merck-ENST00000371489_s_at | MYOF |
| merck-NM_003670_at | BHLHE40 |
| merck-NM_000577_s_at | IL1RN |
| merck-NM_000228_at | LAMB3 |
| merck-NM_003897_a_at | IER3 LINC00243 |
| merck-NM_003955_at | SOCS3 |
| merck-NM_001002857_at | ANXA2 |
| merck-NM_080388_at | S100A16 |
| merck-NM_022162_at | NOD2 |
| merck-NM_003461_at | ZYX |
| merck-NM_002966_at | S100A10 |
| merck-NM_004240_at | TRIP10 |
| merck-NM_005194_at | CEBPB |
| merck-NM_005620_at | S100A11 |
| merck-NM_002090_at | CXCL3 |
| merck-NM_000418_at | IL4R |
| merck-NM_001005377_s_at | PLAUR |
| merck-NM_001005376_at | PLAUR |
| merck-NM_001511_at | CXCL1 |
| merck-BC053563_s_at | MIR21 |
| merck-ENST00000333244_at | AHNAK2 |
| merck2-AI701192_at | LAMC2 |
| merck2-AI701192_x_at | LAMC2 |
| merck2-AI858819_at | — |
| merck2-AK075141_at | RNF149 |
| merck2-AK092006_s_at | — |
| merck2-CA445253_at | MYOF |
| merck2-BT009912_at | — |
| merck2-BT009912_x_at | — |
| merck2-NM_000700_at | ANXA1 |
| merck2-BC001405_at | UPP1 |
| merck2-NM_001005377_at | PLAUR |
| merck2-M62898_x_at | ANXA2 |
| merck2-BG680883_at | — |
| merck2-BC082238_at | BHLHE40 |
| merck2-BG675923_x_at | — |
| merck2-BM543893_x_at | PLAUR |
| merck2-X74039_at | PLAUR |

TABLE 7
Prognosis signature genes

| probe | Gene |
|---|---|
| merck-CN269476_a_at | PCDP1 |
| merck-NM_002126_at | HLF |
| merck-NM_031911_a_at | C1QTNF7 |
| merck2-BX647781_at | C1QTNF7 |
| merck-NM_000901_at | NR3C2 |
| merck-NM_021117_at | CRY2 |
| merck-BU681386_at | SCN7A |
| merck2-AI949138_at | PCDP1 |
| merck-AJ315514_a_at | NR3C2 |
| merck-NM_153267_at | MAMDC2 |
| merck-NM_007037_at | ADAMTS8 |
| merck2-BM684168_at | — |
| merck-NM_006030_at | CACNA2D2 |
| merck-NM_001029996_at | PCDP1 |
| merck-NM_033053_s_at | DMRTC1 DMRTC1B |
| merck2-NM_001080851_s_at | — |
| merck2-BC128418_at | CBX7 |
| merck-AK057720_s_at | OBFC1 |
| merck-NM_002976_at | SCN7A |
| merck-AI027436_at | — |
| merck-AL832580_at | RNF180 |
| merck-NM_004962_at | GDF10 |
| merck-AK124663_a_at | WDFY3-AS2 |
| merck-AF329839_a_at | C1QTNF7 |
| merck2-CB999963_at | RNF180 |
| merck-NM_175709_at | CBX7 |
| merck-NM_007106_at | UBL3 |
| merck-AA129758_a_at | EIF4E3 |
| merck-AK023631_at | — |
| merck2-BC036093_at | HLF |
| merck2-BM976317_at | ANKDD1B |
| merck-BC038509_a_at | RCAN2 |
| merck2-NM_020139_at | BDH2 |
| merck-NM_004469_at | FIGF PIR-FIGF |
| merck-BQ709647_a_at | HLF |
| merck-BG678236_at | SAR1B |
| merck-NM_152606_at | ZNF540 |
| merck-NM_007168_at | ABCA8 |
| merck2-NM_020139_a_at | BDH2 |
| merck2-AL832100_at | ZNF540 |
| merck-AK090989_at | — |
| merck-NM_030569_at | ITIH5 |
| merck-NM_014774_at | EFCAB14 |
| merck-NM_183075_at | CYP2U1 |
| merck-NM_020899_s_at | ZBTB4 |
| merck-BC095414_a_at | BDH2 |
| merck-NM_032411_at | C2orf40 |
| merck2-H45244_at | — |
| merck-NM_006856_at | ATF7 LOC100652999 |
| merck-NM_018488_at | TBX4 |
| merck-NM_018010_at | IFT57 |
| merck-NM_021965_s_at | PGM5 |
| merck2-BC062365_at | SLIT3 |
| merck-NM_172193_at | KLHDC1 |
| merck-NM_005181_at | CA3 |
| merck-CX782760_at | TAPT1 |
| merck-DB366031_s_at | CREBRF |
| merck-NM_199454_at | PRDM16 |
| merck2-AI478811_at | EMCN |
| merck-ENST00000374232_at | SNX30 |
| merck-NM_001008710_s_at | RBPMS |
| merck-NM_152459_at | C16orf89 SEC14L5 |
| merck-AK075495_at | NDFIP1 |
| merck2-CN308012_at | EFCAB14 |
| merck-NM_021977_at | SLC22A3 |
| merck-BX537534_at | BTBD9 |
| merck-NM_001174_s_at | ARHGAP6 |
| merck-AY312852_s_at | GTF2IRD2 GTF2IRD2B GTF2I |
| merck-NM_003206_a_at | TCF21 |
| merck2-NM_001018108_at | SERF2 |
| merck-NM_014880_at | CD302 LY75-CD302 |
| merck-NM_030923_s_at | TMEM163 |
| merck-AL133118_at | EMCN |
| merck2-BG674122_a_at | HLF |
| merck-NM_003099_at | SNX1 CSNK1G1 |
| merck-AL161983_at | EIF4E3 |
| merck2-NM_173537_s_at | — |
| merck-AK130274_at | — |
| merck-BC073920_at | LOC100652999 |
| merck-NM_004614_s_at | TK2 |
| merck-NM_198901_at | SRI |
| merck2-NM_024768_at | EFCC1 |
| merck2-CR598366_at | — |
| merck-NM_014701_at | SECISBP2L |
| merck-ENST00000382101_a_at | DLC1 |
| merck-NM_015328_at | AHCYL2 |
| merck-BX106890_a_at | ITGA8 LOC101928678 |
| merck-BC023330_at | LINC00849 |
| merck-NM_014232_at | VAMP2 |
| merck-BC050653_a_at | NICN1 AMT |
| merck-AK096254_at | — |
| merck-ENST00000283296_a_at | GPR116 LOC101926962 |
| merck2-BX115850_at | IFT57 |
| merck-NM_032866_at | CGNL1 |
| merck-NM_174934_at | SCN4B |
| merck-NM_024513_s_at | FYCO1 |
| merck2-NM_001003795_s_at | — |
| merck-NM_021902_s_at | FXYD1 |
| merck-NM_152913_at | TMEM130 |
| merck-BC030082_at | SORBS2 |

TABLE 8
Proliferation signature genes

| probe | Gene |
|---|---|
| merck-NM_003318_at | TTK |
| merck-NM_014791_at | MELK |
| merck-NM_001786_a_at | CDK1 RHOBTB1 |
| merck-NM_001790_at | CDC25C |
| merck-NM_014176_at | UBE2T |
| merck-BF511624_s_at | BUB1B |
| merck-NM_005030_at | PLK1 |
| merck-NM_181802_at | UBE2C |
| merck-NM_004217_at | AURKB |
| merck-NM_201567_at | CDC25A |
| merck-NM_198436_s_at | AURKA |
| merck-NM_001255_s_at | CDC20 |
| merck-NM_003579_at | RAD54L |
| merck-NM_004336_at | BUB1 RGPD6 |
| merck-NM_031299_at | CDCA3 GNB3 |
| merck-NM_004237_at | TRIP13 |
| merck-BC001459_s_at | RAD51 |
| merck-NM_012484_at | HMMR |
| merck-AB042719_a_at | MCM10 |
| merck-NM_018518_at | MCM10 |
| merck-NM_012291_at | ESPL1 PFDN5 |
| merck-NM_014750_at | DLGAP5 |
| merck-NM_199413_at | PRC1 |
| merck-NM_130398_at | EXO1 |
| merck-NM_199420_s_at | POLQ |
| merck-NM_005733_at | KIF20A CDC23 |
| merck-NM_004856_at | KIF23 |
| merck-NM_004701_at | CCNB2 |
| merck-NM_014321_at | ORC6 |
| merck-NM_002466_at | MYBL2 |
| merck-NM_030919_at | FAM83D |
| merck-NM_003504_at | CDC45 |
| merck-BC075828_a_at | GTSE1 |
| merck-NM_016426_at | GTSE1 TRMU |
| merck-NM_001012409_at | SGOL1 |
| merck-NM_018136_s_at | ASPM |
| merck-NM_018685_at | ANLN |
| merck-NM_012112_at | TPX2 |
| merck-NM_018101_at | CDCA8 |
| merck-NM_001237_a_at | CCNA2 EXOSC9 |
| merck-NM_018454_at | NUSAP1 |
| merck-NM_001211_at | BUB1B |
| merck-U63743_a_at | KIF2C |
| merck-CR596700_a_at | RRM2 |
| merck-NM_012310_at | KIF4A GDPD2 |
| merck-NM_013277_a_at | RACGAP1 |
| merck-NM_018154_at | ASF1B PRKACA |
| merck-BC024211_a_at | NCAPH |
| merck-NM_152515_at | CKAP2L |
| merck-NM_018131_at | CEP55 |
| merck-NM_002417_at | MKI67 |
| merck-CR607300_a_at | MKI67 |
| merck-BI868409_a_at | MKI67 |
| merck-NM_001813_at | CENPE |
| merck-CR602926_s_at | CCNB1 |
| merck-NM_001809_at | CENPA |
| merck-NM_080668_at | CDCA5 |
| merck-AK223428_a_at | BIRC5 |
| merck-NM_005480_at | TROAP |
| merck-NM_021953_at | FOXM1 |
| merck-NM_144508_at | CASC5 |
| merck-NM_019013_at | FAM64A PITPNM3 |
| merck-hCT1776373.2_s_at | DEPDC1 OTUD7A |
| merck-NM_004091_at | E2F2 |
| merck-NM_004219_x_at | PTTG1 |
| merck-NM_002263_a_at | KIFC1 |
| merck-AF331796_a_at | NCAPG |
| merck-NM_145060_at | SKA1 |
| merck-BC048988_a_at | SKA3 |
| merck-NM_152259_s_at | TICRR KIF7 |
| merck-ENST00000243201_a_at | HJURP |
| merck-ENST00000333706_x_at | BIRC5 |
| merck-ENST00000335534_s_at | KIF18B |
| merck-AY605064_at | CLSPN |
| merck2-AK097710_at | CDC25C |
| merck2-AF043294_at | BUB1 RGPD6 |
| merck2-AU132185_at | MKI67 |
| merck2-BC098582_at | KIF14 |
| merck2-BT006759_at | KIF2C |
| merck2-BC006325_at | GTSE1 TRMU |
| merck2-BC006325_x_at | GTSE1 TRMU |
| merck2-AL832036_at | CKAP2L |
| merck2-DQ890621_at | CDC45 |
| merck2-NM_005196_at | CENPF |
| merck2-AV714642_at | ANLN |
| merck2-BC034607_at | ASPM |
| merck2-BC001651_at | CDCA8 |
| merck2-AF098158_at | TPX2 |
| merck2-NM_001168_at | BIRC5 |
| merck2-AK023483_at | NUSAP1 |
| merck2-NM_145061_at | SKA3 |
| merck2-NM_018410_at | HJURP |
| merck2-AL517462_s_at | — |
| merck2-ENST00000333706_s_at | — |
| merck2-BX648516_at | SGOL1 |
| merck2-AK000490_a_at | DEPDC1 |
| merck2-ENST00000370966_a_at | DEPDC1 OTUD7A |
| merck2-AB046790_at | CASC5 |
| merck2-CR936650_at | ANLN |
| merck2-AL519719_a_at | BIRC5 |
| merck2-NM_145060_a_at | SKA1 |
| merck2-NM_001039535_a_at | SKA1 |

The performance of this model was evaluated in reserved validation set of 1,486 samples. FIG. 4 shows the predicted death rate vs. the actual average (running average of 200 samples as ranked by the prediction score) death rate. As shown in the Figure, the model predicts the average death rate very well.

The detailed information about number of samples, number of deaths, and the death rate in each prediction score bin are summarized in Table 9.

TABLE 9
Average death rate versus prediction score.

| Prediction | Number of | Number of | |
|---|---|---|---|
| score | samples | deaths | Rate |
| <0.3 | 151 | 25 | 0.165562914 |
| 0.3-0.4 | 132 | 25 | 0.189393939 |
| 0.4-0.5 | 171 | 68 | 0.397660819 |
| 0.5-0.6 | 207 | 94 | 0.45410628 |
| 0.6-0.7 | 203 | 118 | 0.581280788 |
| 0.7-0.8 | 144 | 82 | 0.569444444 |
| >0.8 | 160 | 122 | 0.7625 |

Using a threshold of 0.4, the odds ratio for overall survival was 5.62 (95% CI: 4.03-7.85), Fisher's Exact Test p-value=2.9×10⁻²⁹.

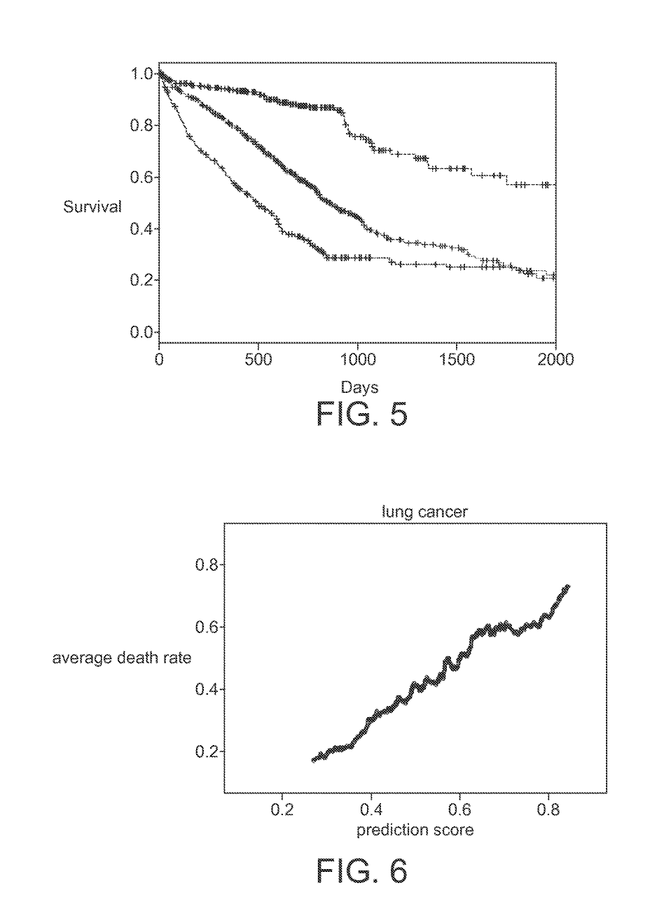

Patients can be further divided into good (risk score<0.4), medium (score 0.4-0.7) and poor (score>0.7) prognosis groups. FIG. 5 shows the Kaplan-Meier curves for these 3 groups. The Chi-square on 2 degrees of freedom is 128 (P=0).

The number of genes in each pathway was reduced to 10 genes.

Immune Signature:
Probe IDs: merck-NM_001767_at, merck2-NM_002209_x_at, merck2-BI519527_at, merck-NM_000732_at, merck2-ENST00000390409_at, merck-NM_014716_at, merck-NM_000733_at, merck-NM_198517_at, merck-NM_000734_at, merck2-NM_052931_at
Gene symbols: CD2, ITGAL, IKZF1, CD3D, TRBC1, ACAP1, CD3E, TBC1D10C, CD247, SLAMF6

Hypoxia:
Probe IDs: merck-NM_006516_at, merck2-BC002829_at, merck-NM_005557_x_at, merck2-NM_005554_at, merck-BX641095_a_at, merck-NM_024009_at, merck-NM_006142_at, merck-NM_033386_s_at, merck-NM_020183_s_at, merck-NM_000094_at
Gene symbols: SLC2A1, S100A2, KRT16, KRT6A, CD109, GJB3, SFN, MICALL1, ARNTL2, COL7A1

Ras Signature:
Probe IDs: merck-NM_005620_at, merck2-AI701192_at, merck2-M62898_x_at, merck-NM_002658_at, merck2-X74039_at, merck-NM_080388_at, merck-NM_000418_at, merck-NM_002068_at, merck-NM_013451_at, merck-NM_000228_at
Gene symbols: S100A11, LAMC2, ANXA2, PLAU, PLAUR, S100A16, IL4R, GNA15, MYOF, LAMB3

Prognosis:
Probe IDs: merck-NM_002126_at, merck-BU681386_at, merck-NM_000901_at, merck2-AI949138_at, merck-NM_007168_at, merck2-AI478811_at, merck-NM_018010_at, merck-BC095414_a_at, merck-NM_153267_at, merck-ENST00000378076_at
Gene symbols: HLF, SCN7A, NR3C2, PCDP1, ABCA8, EMCN, IFT57, BDH2, MAMDC2, ITGA8

Proliferation:
Probe IDs: merck-NM_012112 at merck-NM_001809_at merck-U63743_a_at merck-NM_004701_at merck-NM_080668_at merck-ENST00000243201_a_at merck-NM_012310_at merck-ENST00000333706_x_at merck-NM_014750_at merck-NM_145060_at
Gene symbols: TPX2, CENPA, KIF2C, CCNB2, CDCA5, HJURP, KIF4A, BIRC5, DLGAP5, SKA1

The scores derived from these 10-genes correlated to the original scores at the level of 0.99 for both proliferation and immune scores, 0.98 for ras signature, 0.97 for the prognosis signature and 0.92 for the hypoxia signature.

The ras signature was marginally predictive in the original model, and is not significant after the number of genes was reduced for all these pathways. Hence it was excluded from the model. The formula for the updated model (based on small number of genes) is:

Lung Cancer Risk Score=−0.2853866+(−0.0328615*imscore)+(0.0269496*hscore)+(−0.0006368*prg)+(0.0928468*pscore)+(0.0757314*stage) (Formula 4).

Note, the exact coefficients change depending on the final selection of the technology platform (RNAseq vs. arrays, PCR), and the probe sets or gene lists.

FIG. 6 shows the predicted death rate vs. the actual average (running average of 200 samples as ranked by the prediction score) death rate for this updated model. As shown in the Figure, the model predicts the average death rate very well.

The detailed information about number of samples, number of deaths, and the death rate in each prediction score bin are summarized in Table 10.

TABLE 10
Average death rate versus prediction score.

| Prediction | Number of | Number of | |
|---|---|---|---|
| score | samples | deaths | Rate |
| <0.3 | 141 | 22 | 0.156028369 |
| 0.3-0.4 | 135 | 29 | 0.214814815 |
| 0.4-0.5 | 166 | 60 | 0.361445783 |
| 0.5-0.6 | 220 | 99 | 0.45 |
| 0.6-0.7 | 201 | 116 | 0.577114428 |
| 0.7-0.8 | 140 | 81 | 0.578571429 |
| >0.8 | 165 | 127 | 0.76969697 |

Using a threshold of 0.4, the odds ratio for overall survival was 5.21 (95% CI: 3.74-7.26), Fisher's Exact Test p-value=7.3×10⁻²⁷.

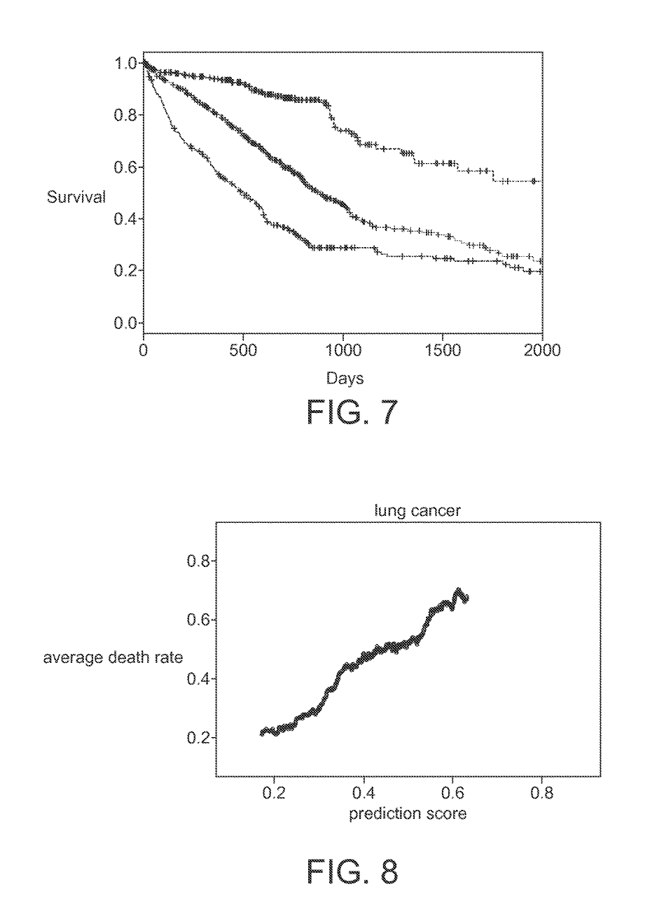

Patients can be further divided into good (risk score<0.4), medium (score 0.4-0.7) and poor (score>0.7) prognosis groups. FIG. 7 shows the Kaplan-Meier curves for these 3 groups. The Chi-square on 2 degrees of freedom is 123 (P=0).

This multicomponent model included both microarray measurement and tumor stage. Each of the components is significant in the model according to the AVOVA analysis in the training set (Table 11).

TABLE 11
ANOVA test of fit model in the training set.

| | Df | Sum Sq | Mean Sq | F value | Pr(>F) |
|---|---|---|---|---|---|
| imscore_f[mke1] | 1 | 5.123 | 5.1230 | 25.269 | 5.664e−07 *** |
| hscore_f[mke1] | 1 | 19.755 | 19.7553 | 97.444 | <2.2e−16 *** |
| prg1_f[mke1] | 1 | 11.888 | 11.8880 | 58.638 | 3.623e−14 *** |
| pscore_f[mke1] | 1 | 11.084 | 11.0838 | 54.671 | 2.509e−13 *** |
| stage[mke1] | 1 | 8.959 | 8.9592 | 44.192 | 4.330e−11 *** |
| Residuals | 1333 | 270.247 | 0.2027 | | |

When microarray components (gene sets) were grouped together using the coefficients from the model, and applied to the validation set, the microarray part of the model was independently predictive of the patient outcome (FIG. 8). The F-static was 142.7 on 1 and 1166 degrees of freedom, P<2×10⁻¹⁶. The tumor stage was also a strong prognostic factor (F-static 103.9 on 1 and 1166 degrees of freedom P<2×10⁻¹⁶).

### Example 3

### Prognostic Model for Colon Cancer

This example describes a colon cancer prognosis model that uses gene expression profiling data and tumor stage. The model contains multiple gene expression signatures as components and the tumor stage. In the second part of the example, the number of genes in each signature is reduced to 10 genes to simplify the implementation of this prognosis model.

There are numerous studies of prognoses using gene expression alone, or histopathology/clinical data alone. Here both are combined to further improve the prognosis.

A total of 2,233 samples were profiled by Affymetrix® expression arrays, among them, 2,203 samples had outcome data (survival vs. death). A composite model was built using the first half of samples and the model validated using the second half of samples. In the first half of samples, 1,091 samples had outcome data (live or death), and 1,076 patients had tumor stage measurement. In the second half of samples, 1,112 had outcome data, and 1,057 patients had stage measurement.

A colon cancer risk model was built in the training set using a general linear model (from the R package) using the following equation:

Colon Cancer Risk Score=−1.109036+(−0.003155 *imscore)+(0.056980*hscore)+(−0.059340*emtscorel)+(−0.040061*emtscore2)+(−0.013334*prg1)+(0.285552*prg2)+(−0.015176*prg3)+(0.084259*stage) (Formula 5),

where “imscore” is an immune score calculated from the immune signature gene in Table 11, “hscore” is a hypoxia score from hypoxia signature genes in Table 13, “emtscorel” is a score from the VIM correlated genes in Table 14, “emtscore2” is a score from the CDH1 correlated genes in Table 15, “prg1” is a score from prognosis genes in Table 16, “prg2” is a score from prognosis genes in Table 17, “prg3” is a score from prognosis genes in Table 18, and “stage” is the composite tumor stage. Scores from the signatures genes were computed simply by averaging the log2 expression level of the genes in the signature.

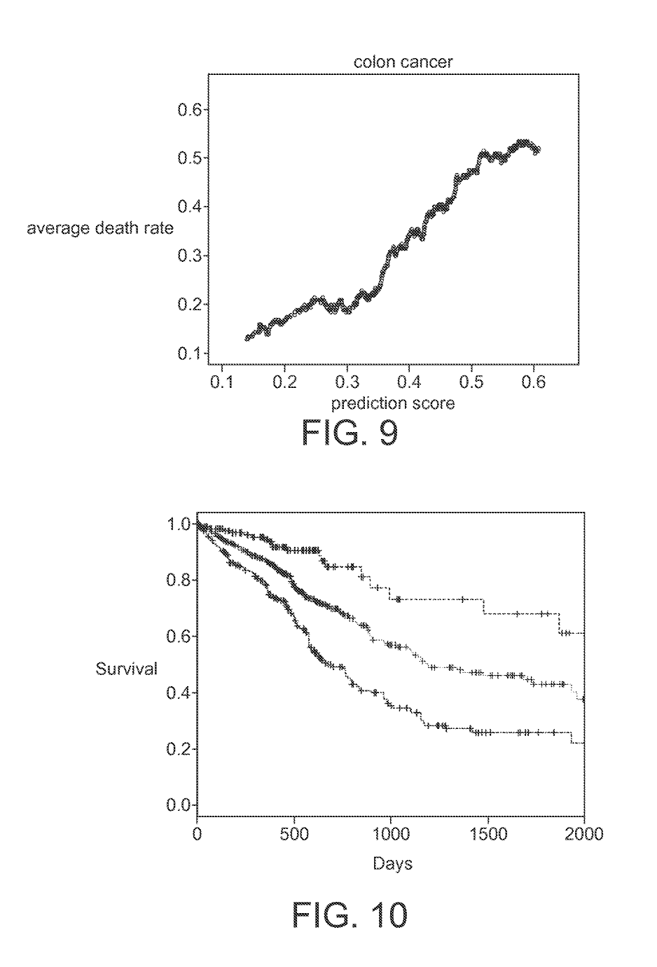

The performance of this model was evaluated using the reserved validation set of 1,057 samples. FIG. 9 shows the predicted death rate vs. the actual average (running average of 200 samples as ranked by the prediction score) death rate. As shown in the Figure, the model predicts the average death rate very well.

The detailed information about number of samples, number of deaths, and the death rate in each prediction score bin are summarized in Table 19.

TABLE 19
Average death rate versus prediction score

| Prediction | Number of | Number of | |
|---|---|---|---|
| score | samples | deaths | Rate |
| <0.2 | 179 | 20 | 0.111731844 |
| 0.2-0.3 | 178 | 39 | 0.219101124 |
| 0.3-0.4 | 194 | 45 | 0.231958763 |
| 0.4-0.5 | 220 | 90 | 0.409090909 |
| >0.5 | 286 | 149 | 0.520979021 |

Using a threshold of 0.48, the odds ratio for overall survival was 3.47 (95% CI: 2.63-4.59), Fisher's Exact Test p-value=1.5×10⁻¹⁷.

Patients can be further divided into good (risk score<0.2), medium (score 0.2-0.5) and poor (score>0.5) prognosis groups. FIG. 10 shows the Kaplan-Meier curves for these 3 groups. The Chi-square on 2 degrees of freedom is 52.6 (P=3.86×10⁻¹²). If the model is applied to the stage 1, 2, 3 patients (excluding stage 4) in the validation set, the Chi-square is 30.5 on 2 degrees of freedom (P=2.3×10⁻⁷, patients in 3 groups, Risk score<0.2, 0.2-0.5 and >0.5). The model is still predictive even if applied to stage 1 & 2 patients in the validation set. The Chi-square is 20.5 on 2 degrees of freedom (P=3.6×10⁻⁵, patients in 3 groups: Risk score<0.2, 0.2-0.4 and >0.4).

The number of genes in each pathway was reduced to 10 genes or less.

Immune signature:
Probe IDs: merck2-BI519527_at, merck2-NM_002209_x_at, merck-NM_001767_at, merck-NM_005546_at, merck-NM_007181_at, merck-NM_000733_at, merck-NM_198517_at, merck-NM_001040067_s_at, merck-NM_000734_at, merck-NM_000732 at
Gene symbols: IKZF1, ITGAL, CD2, ITK, MAP4K1, CD3E, TBC1D10C, TRBC2, CD247, CD3D

Hypoxia:
Probe IDs: merck-NM_006516_at, merck-X15014_a_at, merck-CR614206_a_at, merck-NM_018685_at, merck-NM_005978_at, merck2-AK223027_at, merck-NM_001255_s_at, merck-BG677853_a_at, merck2-X74039_at, merck2-NM001042422_at
Gene symbols: SLC2A1, RALA, ERO1L, ANLN, S100A2, PHLDA2, CDC20, LAMC2, PLAUR, SLC16A3

VIM Correlated Signature:
Probe IDs: merck2-AB266387_s_at,merck2-BQ632060_x_at, merck-ENST00000311127_a_at, merck2-NM_015463_at, merck-NM_006868_at, merck-BU625463_s_at, merck-AK091332_at, merck-NM_012219_s_at, merck-NM_144601_at, merck-NM_003255_s_at
Gene symbols: CCDC80, VIM, HEG1, CNRIP1, RAB31, EFEMP2, GNB4, MRAS, CMTM3, TIMP2

CDH1 Correlated Signature:
Probe IDs: merck-NM_004433_a_at, merck2-NM_001307_at, merck2-NM_001305_at, merck-NM_004360_at, merck-NM_020387_at, merck2-CK818800_at, merck-BC069241_a_at, merck2-NM_001982_at, merck-NM_005498_at, merck-ENST00000378957_a_at
Gene symbols: ELF3, CLDN7, CLDN4, CDH1, RAB25, ESRP1, ESRP2, ERBB3, AP1M2, EPCAM

Prognosis Component 1:
Probe IDs: merck-NM_002126_at, merck-BU681386_at, merck-NM_000901_at, merck2-AI949138_at, merck-NM_007168_at, merck2-AI478811_at, merck-NM_018010_at, merck-BC095414_a_at, merck-NM_153267_at, merck-ENST00000378076_at
Gene symbols: MZB1, OR6C4 IGKV3-11 IGKV3D-11 IGKV3D-20 RHNO1, TNFRSF17, IGKC IGKV1D-39 IGKV1-39, IGHA1 IGHG1 IGH, IGLC1, IGKC IGKV1-16 IGKV1D-16, IGLV6-57, IGLV1-40 IGLV5-39, IGJ

Prognosis Component 2:
Probe IDs: merck2-DQ892544_at, merck2-S42303_at, merck2-NM_133376_a_at, merck-BC010860_a_at, merck-AK125700_a_at, merck2-AL572880_at, merck2-EF043567_at, merck2-AI765059_at, merck2-CB115148_at, merck-NM_003254_at
Gene symbols: SPP1, CDH2, ITGB1, SERPINE1, PLOD2, COL4A1, NTM, MPRIP, PLIN2, TIMP1

The scores derived from these 10-genes correlated to the original scores at the level of 0.99 for both VIM and CDH1 correlated signature scores, and 0.98 for immune signature, 0.90 for the hypoxia signature, 0.99 for the prognosis component 1, and 0.90 for prognosis component 2.

Prognosis component 3 was marginally prognostic in the original model, and was not significant after the signatures reduced to 10 genes, hence was excluded from further models. The formula for the updated model (based on small number of genes) is:

Colon Cancer Risk Score=0.109098+(−0.029915*imscore)+(0.062785*hscore)+(−0.050770*emtscorel)+(−0.042210*emtscore2)+(−0.007858*prgl)+(0.099507*prg2)+(0.088208*stage) (Formula 6).

Note, the exact coefficients will change depending on the final selection of the technology platform (RNAseq vs. arrays, PCR), and the probe sets or gene lists.

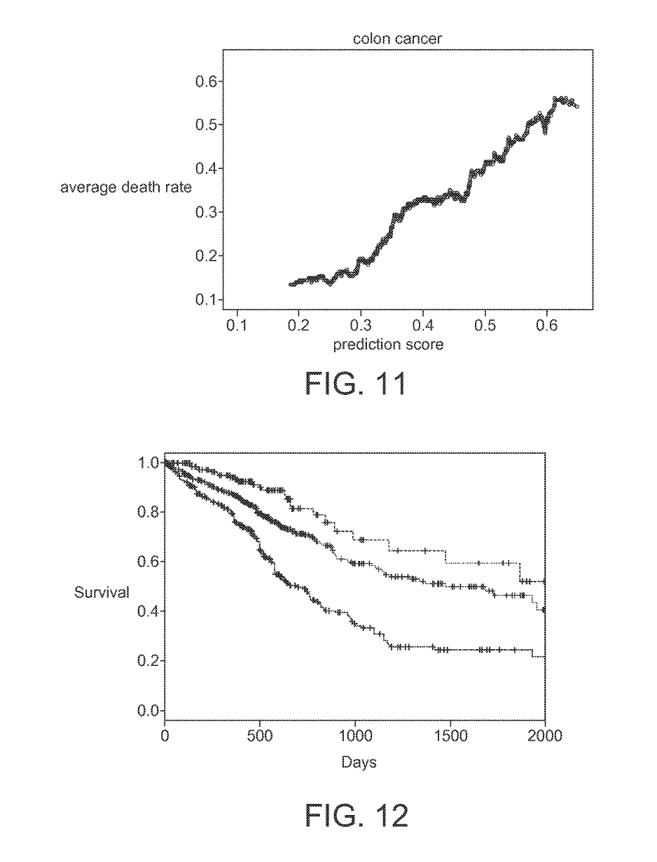

FIG. 11 shows the predicted death rate vs. the actual average (running average of 200 samples as ranked by the prediction score) death rate for this updated model. As shown in the figure, the model predicts the average death rate very well.

The detailed information about number of samples, number of deaths, and the death rate in each prediction score bin are summarized in Table 20.

TABLE 20
Average death rate versus prediction score.

| Prediction | Number of | Number of | |
|---|---|---|---|
| Score | Samples | Deaths | Rate |
| <0.2 | 115 | 13 | 0.113043478 |
| 0.2-0.3 | 148 | 24 | 0.162162162 |
| 0.3-0.4 | 233 | 59 | 0.253218884 |
| 0.4-0.5 | 232 | 82 | 0.353448276 |
| 0.5-0.6 | 175 | 83 | 0.474285714 |
| >0.6 | 154 | 82 | 0.532467532 |

Using a threshold of 0.48, the odds ratio for overall survival was 3.03 (95% CI: 2.31-3.96), Fisher's Exact Test p-value=9.0×10⁻¹⁶.

Patients can be further divided into good (risk score<0.25), medium (score 0.25-0.5) and poor (score>0.5) prognosis groups. FIG. 12 shows the Kaplan-Meier curves for these 3 groups. The Chi-square on 2 degrees of freedom is 57.2 (P=3.7×10⁻¹³).

This multicomponent model included both microarray measurement and tumor stage. Each of the components were significant in the model according to the AVOVA analysis in the training set (Table 21).

TABLE 21
ANOVA test of fit model in the training set.

| | Df | Sum Sq | Mean Sq | F value | Pr(>F) |
|---|---|---|---|---|---|
| imscore_f[mke1] | 1 | 4.070 | 4.0698 | 18.6763 | 1.694e−05 *** |
| hscore_f[mke1] | 1 | 3.738 | 3.7384 | 17.1555 | 3.716e−05 *** |
| emtscore1_f[mke1] | 1 | 4.272 | 4.2722 | 19.6051 | 1.050e−05 *** |
| emtscore2_f[mke1] | 1 | 3.441 | 3.4413 | 15.7923 | 7.544e−05 *** |
| prg1_f[mke1] | 1 | 0.870 | 0.8705 | 3.9946 | 0.0459 * |
| prg2_f[mke1] | 1 | 7.949 | 7.9490 | 36.4783 | 2.128e−09 *** |
| stage[mke1] | 1 | 8.694 | 8.6937 | 39.8956 | 3.924e−10 *** |
| Residuals | 1068 | 232.730 | 0.2179 | | |

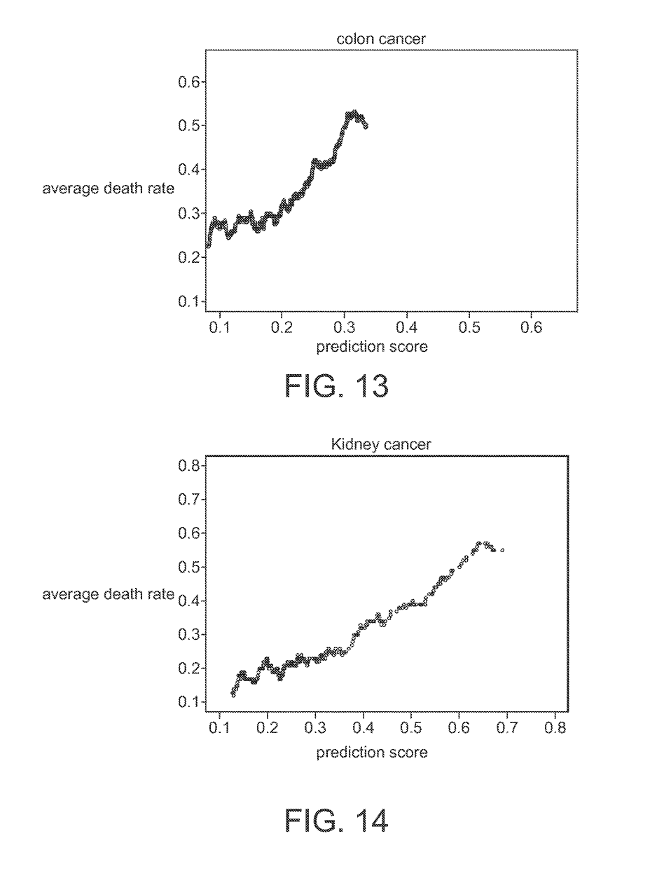

When microarray components (gene sets) were grouped together using the coefficients from the model, and applied to the validation set, the microarray part of the model was independently predictive of the patient outcome (FIG. 13). The F-static is 47.72 on 1 and 1055 degrees of freedom, P=8.5×10⁻¹². The strongest prognostic factor was tumor stage (F-static 84.7 on 1 and 1055 degrees of freedom, P<2×10⁻¹⁶).

TABLE 12
Immune signature genes

| probe | Gene |
|---|---|
| merck-NM_005356_at | LCK |
| merck-NM_006144_at | GZMA |
| merck-NM_014207_at | CD5 |
| merck-NM_005608_at | PTPRCAP |
| merck-NM_007181_at | MAP4K1 |
| merck-NM_002738_at | PRKCB |
| merck-Y00638_s_at | PTPRC |
| merck-BC014239_s_at | PTPRC |
| merck-NM_130446_at | KLHL6 |
| merck-NM_005546_at | ITK CYFIP2 |
| merck-NM_006257_at | PRKCQ |
| merck-NM_002104_at | GZMK |
| merck-NM_001504_at | CXCR3 |
| merck-NM_001001895_at | UBASH3A |
| merck-NM_002832_at | PTPN7 |
| merck-NM_018460_at | ARHGAP15 |
| merck-NM_001838_at | CCR7 |
| merck-NM_002209_at | ITGAL |
| merck-NM_006725_at | CD6 |
| merck-BC028068_s_at | JAK3 INSL3 |
| merck-NM_001079_at | ZAP70 |
| merck-NM_005541_at | INPP5D |
| merck-ENST00000318430_s_at | TMC8 |
| merck-NM_006564_at | CXCR6 |
| merck-NM_007237_s_at | SP140 |
| merck-NM_178129_at | P2RY8 |
| merck-NM_000647_s_at | CCR2 |
| merck-BU428565_s_at | P2RY8 |
| merck-NM_002351_s_at | SH2D1A |
| merck-NM_001040033_at | CD53 |
| merck-NM_005816_at | CD96 |
| merck-NM_198517_at | TBC1D10C |
| merck-NM_000733_at | CD3E |
| merck-NM_002163_at | IRF8 |
| merck-NM_000655_at | SELL |
| merck-NM_003037_at | SLAMF1 |
| merck-NM_003151_a_at | STAT4 |
| merck-NM_001007231_s_at | ARHGAP25 |
| merck-NM_018326_at | GIMAP4 |
| merck-NM_000377_at | WAS |
| merck-NM_001558_at | IL10RA |
| merck-NM_002985_at | CCL5 |
| merck-DT807100_at | CD3D CD3G |
| merck-NM_001465_at | FYB |
| merck-BP339517_a_at | FYB |
| merck-NM_030767_at | AKNA |
| merck-NM_005565_at | LCP2 |
| merck-NM_001040031_at | CD37 |
| merck-NM_002872_at | RAC2 |
| merck-NM_019604_at | CRTAM |
| merck-NM_005263_at | GFI1 |
| merck-NM_001037631_at | CTLA4 ICOS |
| merck-NM_016388_at | TRAT1 |
| merck-NM_014450_at | SIT1 RMRP |
| merck-NM_000732_at | CD3D |
| merck-NM_000073_at | CD3G |
| merck-NM_007360_at | KLRK1 KLRC4-KLRK1 |
| merck-NM_013351_at | TBX21 |
| merck-NM_032214_at | SLA2 |
| merck-NM_000639_at | FASLG |
| merck-NM_001242_at | CD27 |
| merck-ENST00000381961_at | IL7R |
| merck-NM_153206_s_at | AMICA1 |
| merck-NM_001025598_at | ARHGAP30 USF1 |
| merck-NM_001768_at | CD8A |
| merck-NM_003978_at | PSTPIP1 |
| merck-NM_014716_at | ACAP1 |
| merck-AK128740_s_at | IL16 |
| merck-NM_006060_a_at | IKZF1 |
| merck-BC075820_at | IKZF1 |
| merck-NM_016293_at | BIN2 |
| merck-NM_012092_at | ICOS |
| merck-NM_005442_at | EOMES LOC100996624 |
| merck-NM_007074_at | CORO1A |
| merck-NM_000206_at | IL2RG |
| merck-NM_005041_at | PRF1 |
| merck-NM_024898_s_at | DENND1C CRB3 |
| merck-NM_173799_at | TIGIT |
| merck-NM_001767_at | CD2 |
| merck-NM_002348_at | LY9 |
| merck-X60502_s_at | SPN QPRT |
| merck-NM_153236_at | GIMAP7 |
| merck-NM_005601_at | NKG7 |
| merck-NM_032496_at | ARHGAP9 |
| merck-NM_004877_at | GMFG |
| merck-NM_021181_at | SLAMF7 |
| merck-NM_018384_at | GIMAP5 GIMAP1-GIMAP5 |
| merck-NM_181780_at | BTLA |
| merck-NM_001017373_at | SAMD3 |
| merck-NM_000734_at | CD247 |
| merck-NM_003650_at | CST7 |
| merck-NM_172101_at | CD8B |
| merck-NM_001803_at | CD52 |
| merck-NM_001778_at | CD48 |
| merck-NM_001025265_at | CXorf65 |
| merck-NM_198929_at | PYHIN1 |
| merck-ENST00000379833_at | GVINP1 |
| merck-NM_052931_at | SLAMF6 |
| merck-NM_001024667_s_at | FCRL3 |
| merck-NM_002258_at | KLRB1 |
| merck-NM_018556_s_at | SIRPG |
| merck-AK090431_s_at | NLRC3 |
| merck-NM_018990_at | SASH3 XPNPEP2 |
| merck-NM_175900_s_at | C16orf54 QPRT |
| merck-ENST00000316577_s_at | TESPA1 |
| merck-NM_024070_at | PVRIG |
| merck-AY190088_s_at | — |
| merck-NM_001040067_s_at | TRBC2 TRBV3-1 TRBV5-4 |
| | TRBV6-5 TRBV7-2 |
| merck-NM_130848_s_at | C5orf20 |
| merck-ENST00000381153_at | C11orf21 |
| merck-ENST00000382913_s_at | TRAC TRAJ17 TRAV20 TRDV2 |
| merck-BC030533_s_at | TRBC1 TRBV19 |
| merck-ENST00000244032_a_at | ZNF831 |
| merck-ENST00000371030_at | ZNF831 |
| merck-ENST00000343625_s_at | RASAL3 |
| merck-AF143887_at | — |
| merck-AK128436_at | IKZF3 |
| merck-AI281804_at | GPR174 |
| merck-AF086367_at | — |
| merck-CR598049_at | LINC00426 |
| merck-BM700951_at | KLRK1 KLRC4-KLRK1 |
| merck-BX648371_at | LINC00861 |
| merck-BC070382_at | — |
| merck2-AW798052_at | AKNA |
| merck2-BX640915_at | TIGIT |
| merck2-BM678246_at | CD37 |
| merck2-NM_025228_at | TRAF3IP3 |
| merck2-XM_033379_at | WDFY4 |
| merck2-AJ515553_at | AMICA1 |
| merck2-BP262340_at | IL16 |
| merck2-AK225623_at | DENND1C CRB3 |
| merck2-AL833681_at | CD96 |
| merck2-BF111803_at | ARHGAP15 |
| merck2-BX406128_at | CD3G |
| merck2-NM_153701_at | — |
| merck2-BC020657_at | GIMAP4 |
| merck2-AY185344_at | PYHIN1 |
| merck2-DR159064_at | EOMES LOC100996624 |
| merck2-ENST00000390420_at | TRBV3-1 TRBV5-4 TRBV6-5 |
| | TRBV7-2 |
| merck2-ENST00000390420_s_at | — |
| merck2-NM_001010923_at | THEMIS |
| merck2-ENST00000390409_at | TRBC1 TRBV19 |
| merck2-AX721088_at | — |
| merck2-ENST00000390393_at | TRBV19 |
| merck2-AW341086_at | — |
| merck2-AA278761_at | — |
| merck2-AA278761_x_at | — |
| merck2-ENST00000390394_s_at | — |
| merck2-AA669142_at | — |
| merck2-AW007991_at | PTPRC |
| merck2-BG743900_at | PRKCB |
| merck2-X06318_at | PRKCB |
| merck2-BI519527_at | IKZF1 |
| merck2-ENST00000390537_s_at | — |
| merck2-AY292266_x_at | — |
| merck2-NM_005816_a_at | CD96 |
| merck2-NM_198196_a_at | CD96 |
| merck2-NM_001114380_x_at | ITGAL |
| merck2-NM_007237_a_at | SP140 |
| merck2-NM_007237_at | SP140 |
| merck2-NM_052931_at | SLAMF6 |
| merck2-NM_001558_at | IL10RA |
| merck2-NM_007360_at | KLRK1 KLRC4-KLRK1 |
| merck2-NM_002209_x_at | ITGAL |
| merck2-NM_175900_at | C16orf54 QPRT |

TABLE 13
Hypoxia signature genes

| probe | Gene |
|---|---|
| merck-NM_002627_at | PFKP PITRM1 |
| merck-NM_000302_at | PLOD1 |
| merck-NM_001216_at | CA9 RMRP |
| merck-ENST00000377093_at | KIF1B |
| merck-BC004202_a_at | CHEK1 |
| merck-NM_030949_at | PPP1R14C |
| merck-CR593119_a_at | CLIC4 |
| merck-NM_001255_s_at | CDC20 |
| merck-BG679113_s_at | KRT6A KRT6B KRT6C |
| merck-NM_002421_at | MMP1 |
| merck-BQ217236_a_at | SERPINB5 |
| merck-NM_001793_at | CDH3 |
| merck-NM_001238_at | CCNE1 |
| merck-BU597348_s_at | SYNCRIP |
| merck-NM_006516_at | SLC2A1 |
| merck-BX648425_a_at | DSC2 |
| merck-X15014_a_at | RALA |
| merck-NM_018685_at | ANLN |
| merck-CR614206_a_at | ERO1L |
| merck-NM_001124_at | ADM |
| merck-NM_015440_at | MTHFD1L |
| merck-ENST00000367307_a_at | MTHFD1L |
| merck-NM_058179_at | PSAT1 |
| merck-NM_031415_s_at | GSDMC |
| merck-NM_005557_x_at | KRT16 |
| merck-NM_053016_at | PALM2 PALM2-AKAP2 |
| merck-CR602579_a_at | CTPS1 |
| merck-NM_001428_s_at | ENO1 |
| merck-ENST00000305850_at | CENPN CMC2 |
| merck-NM_005978_at | S100A2 |
| merck-NM_018643_at | TREM1 |
| merck-NM_006505_at | PVR |
| merck-NM_080655_s_at | MSANTD3 |
| merck-NM_001012507_at | CENPW |
| merck-ENST00000258005_a_at | NHSL1 |
| merck-AK129763_at | LINC00673 |
| merck-XM_927868_s_at | PGK1 |
| merck-XM_928117_x_at | FAM106B |
| merck-AL359337_at | ADM |
| merck-AA148856_s_at | SYNCRIP |
| merck2-AI989728_at | SERPINB5 |
| merck2-DQ892208_at | CA9 RMRP |
| merck2-AK022036_at | WWTR1 |
| merck2-AA677426_at | — |
| merck2-AA677426_s_at | — |
| merck2-BC004856_at | NCS1 |
| merck2-BG252150_at | PFKP |
| merck2-BC007633_at | AGO2 |
| merck2-BG400371_at | — |
| merck2-DQ891441_at | — |
| merck2-NM_017522_AS_at | LRP8 |
| merck2-AF039652_at | RNASEH1 |
| merck2-AV714642_at | ANLN |
| merck2-AB030656_at | CORO1C |
| merck2-NM_000291_at | PGK1 |
| merck2-NM_005554_at | KRT6A |
| merck2-BC002829_at | S100A2 |
| merck2-BU681245_at | — |
| merck2-AK225899_a_at | CTPS1 |
| merck2-BC062635_a_at | XPO5 |
| merck2-AF257659_a_at | CALU |
| merck2-CA308717_at | — |
| merck2-X56807_at | DSC2 |
| merck2-CR936650_at | ANLN |
| merck2-AY423725_a_at | PGK1 |
| merck2-BC103752_a_at | PGK1 |

TABLE 14
VIM correlated genes

| probe | Gene |
|---|---|
| merck-NM_005211_at | CSF1R |
| merck-NM_001699_at | AXL |
| merck-NM_032525_at | TUBB6 |
| merck-AL710269_a_at | CDK14 |
| merck-NM_152653_s_at | UBE2E2 |
| merck-NM_032777_s_at | GPR124 |
| merck-AF085983_s_at | ZEB2 |
| merck-NM_002510_at | GPNMB |
| merck-NM_002444_at | MSN |
| merck-NM_016938_at | EFEMP2 |
| merck-NM_031934_at | RAB34 |
| merck-NM_016815_at | GYPC |
| merck-NM_005429_at | VEGFC |
| merck-NM_003380_a_at | VIM |
| merck-ENST00000316623_a_at | FBN1 |
| merck-NM_003873_at | NRP1 |
| merck-BU625463_s_at | EFEMP2 |
| merck-NM_003255_s_at | TIMP2 |
| merck-CA447839_at | FAM49A |
| merck-AY548106_a_at | CCDC80 |
| merck-BC086876_a_at | CCDC80 |
| merck-NM_006317_at | BASP1 |
| merck-NM_006832_at | FERMT2 |
| merck-NM_003118_s_at | SPARC |
| merck-NM_005461_at | MAFB |
| merck-NM_013352_at | DSE |
| merck-NM_002017_at | FLI1 |
| merck-NM_020856_at | TSHZ3 |
| merck-NM_014737_at | RASSF2 |
| merck-NM_014795_at | ZEB2 |
| merck-BC025730_at | ZEB2 |
| merck-NM_144601_at | CMTM3 |
| merck-NM_016429_at | COPZ2 |
| merck-NM_012219_s_at | MRAS |
| merck-NM_001425_at | EMP3 TMEM143 |
| merck-NM_012072_at | CD93 |
| merck-NM_016274_s_at | PLEKHO1 |
| merck-NM_206853_s_at | QKI |
| merck-NM_006868_at | RAB31 |
| merck-DB025966_a_at | RAB31 |
| merck-AL833176_at | CHST11 |
| merck-AF055376_at | MAF LOC101928230 |
| merck-CR616358_s_at | DCN |
| merck-NM_001031679_at | MSRB3 |
| merck-CR604988_a_at | CLEC2B |
| merck-NM_015150_at | RFTN1 |
| merck-NM_052966_at | FAM129A |
| merck-NM_024579_at | C1orf54 |
| merck-XM_087386_at | HEG1 |
| merck-ENST00000311127_a_at | HEG1 |
| merck-ENST00000252031_at | C20orf194 |
| merck-ENST00000252032_a_at | C20orf194 |
| merck-AK123315_a_at | LOC100132891 |
| merck-AK091332_at | GNB4 |
| merck2-AF086016_at | NRP1 |
| merck2-NM_199511_at | CCDC80 |
| merck2-NM_003768_at | PEA15 |
| merck2-BC010410_at | TIMP2 |
| merck2-BM468535_at | — |
| merck2-BC023509_at | CMTM3 |
| merck2-G43223_a_at | VIM |
| merck2-NM_001920_at | DCN |
| merck2-NM_015463_at | CNRIP1 |
| merck2-CB240675_at | — |
| merck2-AA664657_x_at | VIM |
| merck2-BX352133_s_at | — |
| merck2-BM754248_at | FBN1 |
| merck2-AB266387_s_at | CCDC80 |
| merck2-AK075210_a_at | CCDC80 |
| merck2-CX871427_at | BASP1 |
| merck2-DQ892556_a_at | DCN LOC101928584 |
| merck2-BQ632060_x_at | VIM |
| merck2-BM999558_x_at | VIM |

TABLE 15
CDH1 correlated genes

| probe | Gene |
|---|---|
| merck-NM_002773_at | PRSS8 |
| merck-NM_020770_at | CGN |
| merck-M34309_a_at | ERBB3 |
| merck-NM_002273_x_at | KRT8 |
| merck-NM_004360_at | CDH1 TANGO6 |
| merck-NM_024729_s_at | MYH14 KCNC3 |
| merck-NM_052886_at | MAL2 |
| merck-BC069241_a_at | ESRP2 |
| merck-NM_002670_at | PLS1 |
| merck-NM_004433_a_at | ELF3 |
| merck-ENST00000367284_at | ELF3 |
| merck-NM_001034915_s_at | ESRP1 |
| merck-BC016153_s_at | TMEM45B |
| merck-BX364926_at | IRF6 |
| merck-NM_006147_at | IRF6 |
| merck-ENST00000378957_a_at | EPCAM |
| merck-NM_001305_at | CLDN4 |
| merck-NM_007183_at | PKP3 |
| merck-NM_001008844_at | DSP |
| merck-NM_020387_at | RAB25 |
| merck-NM_173853_s_at | KRTCAP3 |
| merck-NM_005498_at | AP1M2 |
| merck-NM_199187_x_at | KRT18 |
| merck-NM_001017967_at | MARVELD3 PHLPP2 |
| merck-NM_000346_at | SOX9 |
| merck-NM_024320_at | PRR15L |
| merck-NM_001307_at | CLDN7 |
| merck-NM_144724_at | MARVELD2 |
| merck-NM_173481_t | MISP |
| merck-AK093149_a_at | MYO5B |
| merck-AK026517_at | EHF |
| merck-CB160685_s_at | HNF4A |
| merck-AF086028_at | ERBB3 |
| merck2-NM_001982_at | ERBB3 |
| merck2-AI052130_at | TMEM45B |
| merck2-CK818800_at | ESRP1 |
| merck2-AB209992_at | DSP |
| merck2-CN341876_at | IRF6 GRM7 |
| merck2-NM_002354_at | EPCAM |
| merck2-NM_001305_at | CLDN4 |
| merck2-NM_199187_x_at | — |
| merck2-NM_001307_at | CLDN7 |
| merck2-BE542388_at | CDH1 TANGO6 |
| merck2-AK025901_a_at | ESRP2 |
| merck2-CA314539_at | NFATC3 |
| merck2-BM981128_at | — |
| merck2-ENST00000367021_at | IRF6 |
| merck2-AJ011497_a_at | CLDN7 |
| merck2-NM_182517_at | C1orf210 |

TABLE 16
Prognosis component 1 (prg1) genes

| Probe | Gene |
|---|---|
| merck-NM_001192_at | TNFRSF17 |
| merck-NM_144646_at | IGJ |
| merck2-AF343666_at | — |
| merck2-DQ884395_a_at | IGJ |
| merck-NM_016459_at | MZB1 |
| merck2-AK125079_s_at | — |
| merck2-BX648616_s_at | — |
| merck-NM_006235_at | POU2AF1 |
| merck-AX747748_s_at | IGHA1 IGHA2 IGH |
| merck2-BC020889_at | IGJ |
| merck2-BF174271_at | MZB1 |
| merck-NM_001783_at | CD79A |
| merck2-BC007782_at | IGLC1 |
| merck2-U52682_at | IRF4 |
| merck-NM_006875_at | PIM2 |
| merck-ENST00000290730_s_at | DERL3 |
| merck2-ENST00000304187_x_at | — |
| merck2-ENST00000390629_x_at | — |
| merck-ENST00000379877_x_at | IGHA1 IGHG1 IGH |
| merck2-ENST00000390243_x_at | — |
| merck-AF343662_at | FCRL5 |
| merck2-ENST00000390290_x_at | — |
| merck-BC070352_x_at | IGLV3-21 |
| merck2-XM_037686_at | DERL3 |
| merck-ENST00000241813_at | TNFRSF17 |
| merck-NM_014879_at | P2RY14 |
| merck2-ENST00000390273_x_at | IGKC IGKV1-16 IGKV1D-16 |
| merck2-ENST00000390243_at | — |
| merck-NM_017709_at | FAM46C |
| merck2-DB327580_at | FCRL5 |
| merck2-ENST00000379900_x_at | — |
| merck2-ENST00000390290_at | — |
| merck-AF035036_x_at | IGK IGKV3-20 IGKV3D-20 |
| merck-BC042060_x_at | LOC100509541 |
| merck2-ENST00000390615_x_at | — |
| merck2-L37307_x_at | — |
| merck-ENST00000333289_x_at | IGLV6-57 |
| merck-U07440_x_at | OR6C4 IGKV3-11 IGKV3D-11 IGKV3D-20 RHNO1 |
| merck-AK091834_at | FENDRR |
| merck-X57809_x_at | — |
| merck2-ENST00000390615_at | — |
| merck2-U07440_x_at | — |
| merck2-ENST00000390630_x_at | — |
| merck-AK024399_at | TSPAN11 |
| merck2-CD703280_at | IGKC IGK IGKV3-11 IGKV3-20 IGKV3D-20 |
| merck2-BE935035_at | — |
| merck2-NM_017773_at | LAX1 |
| merck-NM_001242_at | CD27 |
| merck-ENST00000360329_at | KIAA0125 |
| merck2-ENST00000359488_x_at | IGKC IGKV1D-39 IGKV1-39 |
| merck2-ENST00000390272_x_at | IGKV1D-17 |
| merck2-Z47250 x_at | — |
| merck-NM_017773_at | LAX1 |
| merck-CR605298_s_at | FENDRR |
| merck2-AF408729_x_at | IGKC IGKV2-30 IGKV2D-30 |
| merck-NM_002460_at | IRF4 |
| merck-ENST00000382880_x_at | CYAT1 IGLL5 IGLC1 IGLC2 IGLC3 IGLJ3 IGLV1- |
| | 44 IGLV3-25 IGLV4-3 |
| merck2-S67637_x_at | — |
| merck2-AF035036_x_at | IGKV3-20 |
| merck-ENST00000304187_x_at | IGK IGKV1-5 IGKV3-15 IGKV3D-15 |
| merck2-ENST00000390299_x_at | IGLV1-40 IGLV5-39 |
| merck-BC022823_x_at | IGLV3-25 |
| merck-NM_014792_at | KIAA0125 |
| merck2-BC022823_x_at | IGLV3-25 |
| merck-NM_003037_at | SLAMF1 |
| merck-NM_021181_at | SLAMF7 |
| merck-NM_031281_at | FCRL5 |
| merck-NM_001775_at | CD38 |
| merck-NM_000036_at | AMPD1 |
| merck2-ENST00000390276_x_at | — |
| merck2-ENST00000390285_at | IGLV6-57 |
| merck-ENST00000358611_x_at | IGKC IGKV1D-16 |
| merck-DB350188_a_at | IGHG1 IGHG3 IGHM |
| merck-NM_001002862_at | DERL3 SMARCB1 |
| merck-AI676062_at | TCONS_00024492 LOC101928582 LOC146513 |
| | TCONS_00024764 |
| merck-AJ004955_at | IGKV4-1 |
| merck2-BC009851_at | IGHM |
| merck-AK097071_s_at | IGHM |
| merck-AAS02609_a_at | TRPA1 |
| merck2-CR749861_x_at | — |
| merck2-ENST00000390265_x_at | IGKC IGKV1-33 IGKV1D-33 |
| merck-NM_145285_s_at | NKX2-3 |
| merck-NM_020939_at | CPNE5 |
| merck2-M34461_at | CD38 |
| merck2-ENST00000379894_x_at | — |
| merck-ENST00000331195_x_at | — |
| merck-NM_002986_s_at | CCL11 |
| merck2-S67987_x_at | — |
| merck2-AF076199_at | — |
| merck2-XM_001133802_at | LOC101928582 TCONS_00024492 LOC146513 |
| | TCONS_00024764 |
| merck-ENST00000359488_x_at | IGKV1D-39 IGKV@ IGKV1-39 |
| merck-X57817_x_at | IGLJ3 |
| merck2-AF076199_x_at | — |
| merck-ENST00000379884_x_at | IGHG1 IGHV1-46 |
| merck-L43092_x_at | CKAP2 IGLJ3 IGLV3-19 |
| merck-BX648045_s_at | ANKRD36B |
| merck2-BC017850_at | CCL11 |
| merck-NM_030764_s_at | FCRL2 |
| merck2-ENST00000390593_at | IGHM IGHV6-1 |
| merck2-Z14216_x_at | IGHV3-15 |

TABLE 17
Prognosis component 2 (prg2) genes

| probe | Gene |
|---|---|
| merck-NM_001017962_at | P4HA1 |
| merck2-BX648829_at | P4HA1 |
| merck2-DQ892544_at | SPP1 |
| merck2-AK124671_a_at | TMCC1 |
| merck-BC039859_a_at | TMCC1 |
| merck2-BM985119_a_at | VEGFA |
| merck-NM_000582_at | SPP1 |
| merck-ENST00000373907_a_at | DLGAP4 |
| merck-ENST00000199940_a_at | MAP2 |
| merck-AK021681_a_at | SEPT10 |
| merck2-Z29328_a_at | UBE2H |
| merck-BP311362_a_at | LUZP6 MTPN |
| merck-NM_181552_at | CUX1 |
| merck-AF125392_a_at | INSIG2 |
| merck2-BE900907_a_at | UBE2H |
| merck-NM_054034_a_at | FN1 |
| merck-NM_199235_at | COLEC11 |
| merck-X54315_a_at | CDH2 |
| merck2-BQ277651_at | CDH2 |
| merck-AK125666_a_at | VEGFA |
| merck-NM_002182_at | IL1RAP |
| merck2-AF277174_at | EGLN1 |
| merck-AF028828_at | SNTB1 |
| merck-DA993973_a_at | KBTBD2 |
| merck-ENST00000377499_a_at | LMO7 |
| merck-BF056045_a_at | MPRIP |
| merck-CR612713_s_at | MAPK14 |
| merck-AK056350_s_at | DCBLD2 |
| merck2-AI765059_at | MPRIP |
| merck2-CB115148_at | PLIN2 |
| merck-ENST00000367307_a_at | MTHFD1L |
| merck2-NM_133376_a_at | ITGB1 |
| merck-BG706780_s_at | RHEB |
| merck2-BG699831_at | INSIG2 |
| merck-ENST00000369578_a_at | ZNF292 |
| merck2-DB483456_at | YWHAG |
| merck-NM_053043_at | RBM33 |
| merck-NM_022347_at | TOR1AIP2 |
| merck2-BX647140_at | DCBLD2 |
| merck2-AA446940_at | DLGAP4 |
| merck-BU538528_s_at | MAP2 |
| merck2-DB498046_x_at | HSP90AB1 |
| merck-BC010860_a_at | SERPINE1 |
| merck-ENST00000382881_a_at | ZMYM2 |
| merck2-S42303_at | CDH2 |
| merck-AK125700_a_at | PLOD2 |
| merck2-BQ000301_at | NAB1 LOC101927315 |
| merck-NM_177444_s_at | PPFIBP1 |
| merck-M94010_a_at | F5 |
| merck-AK057337_at | LINC00924 |
| merck2-BE669868_a_at | ANKLE2 |
| merck-ENST00000376200_s_at | NALCN |
| merck2-AF322916_at | UACA LOC101929151 |
| merck-BQ440605_a_at | ITGB1 |
| merck-DB226799_a_at | PTK2 |
| merck-NM_006516_at | SLC2A1 |
| merck-CR624299_s_at | GRB10 |
| merck-AK000990_a_at | UACA |
| merck2-NM_178826_at | ANO4 UTP20 |
| merck-NM_005401_at | PTPN14 |
| merck-BX640712_a_at | TMCC1 |
| merck-BX451561_a_at | ARHGEF7 |
| merck-AF075090_a_at | MET |
| merck-BI917224_a_at | PLIN2 |
| merck-DA409370_a_at | MAP4K3 |
| merck2-AW162846_at | — |
| merck-NM_001084_at | PLOD3 |
| merck2-CA423142_a_at | MLLT4 KIF25 |
| merck2-DB498046_at | HSP90AB1 |
| merck2-NM_000908_at | NPR3 |
| merck-NM_015852_at | ZNF117 |
| merck-NM_000908_at | NPR3 |
| merck-NM_001792_a_at | CDH2 |
| merck2-BC018124_at | HSPH1 |
| merck-NM_021175_at | HAMP |
| merck-BC065279_a_at | IWS1 |
| merck-BC001136_a_at | PLEKHA1 |
| merck-AV717806_a_at | HSPH1 |
| merck2-M16967_at | F5 |
| merck-NM_018433_s_at | KDM3A |
| merck2-BQ217998_a_at | ANKLE2 |

TABLE 18
Prognosis component 3 genes

| probe | Gene |
|---|---|
| merck-NM_001013029_at | IGFBP1 |
| merck-BG567539_a_at | FGA |
| merck2-NM_021871_at | FGA |
| merck2-BC106760_at | FGB |
| merck-NM_005141_at | FGB |
| merck2-AI174982_at | FGB |
| merck-NM_000509_at | FGG |
| merck2-NM_021870_at | FGG |
| merck-NM_002216_at | ITIH2 |
| merck2-BC007058_at | APCS |
| merck-NM_001639_at | APCS |
| merck2-NM_000567_at | CRP |
| merck-NM_000567_at | CRP |
| merck-NM_000583_at | GC |
| merck2-AV645562_a_at | ALB |
| merck2-U22961_a_at | ALB |
| merck2-AF119840_at | ALB |
| merck2-DQ891414_x_at | ALB |
| merck2-AY960291_x_at | ALB |

### Example 4

### Prognostic Model for Kidney Cancer

This example describes a kidney cancer prognosis model based on gene expression profiling data. The model contains two gene expression signatures as components. In the second part of the example, the number of genes in each signature is reduced to 10 genes to simplify the implementation of this prognosis model.

A total of 893 samples were profiled by Affymetrix® expression arrays. A composite model was built using the first half of samples and the model was validated using the second half of samples. In the first half of samples, 443 samples had outcome data (live or death). In the second half of samples, 444 had outcome data. The detailed last follow-up dates for the good outcome patients are incomplete. In the first half of samples, 106 out of 283 good outcome patients did not have the last follow-up date. In the second half of samples, 146/315 good outcome patients did not have the last follow-up date. In poor outcome patients, all but one had last follow-up dates.

Two groups of genes (100 Affymetrix® probe-sets each) were identified in 443 training samples which are either correlated or anti-correlated with poor outcome. These two groups of genes are displayed in Tables 22 & 23. Genes in Table 23 are highly enriched for cell cycle and cell proliferation pathways.

TABLE 22
Prognosis signature component 1
(anti-correlated with poor outcome) genes

| probe | Gene |
|---|---|
| merck-NM_000901_at | NR3C2 |
| merck-M13994_a_at | BCL2 |
| merck2-BM977883_at | FAM221B |
| merck-NM_021117_at | CRY2 |
| merck-NM_001280_a_at | CIRBP |
| merck2-BC036093_at | HLF |
| merck-NM_018945_s_at | PDE7B |
| merck-NM_138333_at | FAM122A |
| merck-BQ709647_a_at | HLF |
| merck-NM_014014_at | SNRNP200 |
| merck2-AF316873_at | PINK1 DDOST |
| merck-H05603_a_at | THRA NR1D1 |
| merck2-NM_182517_at | C1orf210 |
| merck2-AB075482_at | — |
| merck2-BF433548_at | — |
| merck2-NM_003250_at | — |
| merck-NM_025202_at | EFHD1 |
| merck-NM_182517_at | C1orf210 |
| merck2-CK005338_at | — |
| merck-ENST00000375138_s_at | MINOS1 |
| merck2-NM_003250_a_at | THRA NR1D1 |
| merck-ENST00000377991_at | TMEM8B FAM221B |
| merck-ENST00000269197_at | ASXL3 |
| merck2-BG674122_a_at | HLF |
| merck-ENST00000264431_s_at | RAPGEF2 |
| merck-NM_014234_a_at | HSD17B8 |
| merck-NM_015316_at | PPP1R13B |
| merck2-BU159596_at | BCL2 |
| merck-NM_024563_at | NPR3 |
| merck-ENST00000307249_at | EPB41L4A-AS2 |
| merck-NM_000633_at | BCL2 |
| merck-AY117034_a_at | EMX2OS |
| merck-NM_201536_s_at | NDRG2 |
| merck-NM_175709_at | CBX7 |
| merck2-BF940198_at | LIFR-AS1 LIFR |
| merck-AJ315514_a_at | NR3C2 |
| merck-NM_002126_at | HLF |
| merck2-AF070541_at | LOC284244 |
| merck-BX335786_s_at | FAM47E |
| merck-AK126966_at | TADA2B |
| merck2-BC128418_at | CBX7 |
| merck-BC063296_at | MTMR10 FAN1 |
| merck2-BX408834_at | NDRG2 |
| merck-NM_080597_at | OSBPL1A |
| merck2-AK021580_at | PPP1R13B |
| merck-NM_014828_at | TOX4 METTL3 |
| merck-NM_017719_at | SNRK |
| merck-NM_032385_at | FAXDC2 |
| merck2-AW612403_at | CCDC176 ALDH6A1 |
| merck-BX437500_at | SCAI |
| merck-NM_000908_at | NPR3 |
| merck-NM_145689_s_at | APBB1 SMPD1 |
| merck-NM_004928_at | C21orf2 |
| merck2-NM_030807_at | SLC2A11 |
| merck2-AI927896_at | — |
| merck-BG536817_a_at | TMEM245 |
| merck2-NM_000908_at | NPR3 |
| merck-NM_001042_at | SLC2A4 |
| merck-ENST00000332811_at | ZNRF3 |
| merck-NM_024900_at | PHF17 |
| merck-AK091971_a_at | PKHD1 |
| merck-NM_006393_at | NEBL |
| merck-NM_031889_at | ENAM |
| merck-AK021616_at | OTUD7A |
| merck-BC038509_a_at | RCAN2 |
| merck-AK123831_at | CDS2 |
| merck2-NM_003991_at | EDNRB |
| merck-ENST00000344980_s_at | ZNF433 |
| merck2-DQ890997_a_at | APBB1 |
| merck-NM_013381_at | TRHDE |
| merck-AK001936_a_at | EIF4EBP2 |
| merck-BC095414_a_at | BDH2 |
| merck-NM_032717_at | AGPAT9 |
| merck-ENST00000377448_a_at | ZNF204P |
| merck-AK021522_a_at | VAMP2 |
| merck2-AW966622_at | NEBL |
| merck2-ENST00000377187_at | NEBL |
| merck-BC014248_a_at | TMEM245 |
| merck-AB007969_at | CLMN |
| merck-NM_001979_at | EPHX2 |
| merck-BM925725_a_at | LIFR |
| merck-NM_153281_s_at | HYAL1 |
| merck2-AA043801_at | SYNJ2BP |
| merck-NM_032233_at | SETD3 BCL11B |
| merck-NM_004098_s_at | EMX2 |
| merck2-BF945736_at | C21orf2 |
| merck2-XM_085862_s_at | ILF3-AS1 |
| merck-DA383742_a_at | EMX2OS |
| merck-NM_182758_at | WDR72 |
| merck2-NM_023926_a_at | ZSCAN18 |
| merck-BC042390_s_at | VTI1B |
| merck-NM_021229_at | NTN4 |
| merck-NM_152444_at | PTGR2 |
| merck2-BU687744_at | — |
| merck-NM_020698_at | TMCC3 |
| merck2-BC032376_at | PHF17 |
| merck-NM_030911_at | CDADC1 |
| merck2-AI761584_at | — |
| merck2-BC034387_at | SLC2A4 |
| merck-AK055143_s_at | — |

TABLE 23
Prognosis signature component 2 (correlated with poor outcome) genes

| probe | Gene |
|---|---|
| merck2-AF043294_at | BUB1 RGPD6 |
| merck-NM_004336_at | BUB1 RGPD6 |
| merck-NM_005733_at | KIF20A CDC23 |
| merck2-NM_005196_at | CENPF |
| merck-NM_012112_at | TPX2 |
| merck-NM_181802_at | UBE2C |
| merck-NM_001809_at | CENPA |
| merck2-BC006325_at | GTSE1 TRMU |
| merck-NM_004701_at | CCNB2 |
| merck2-AF098158_at | TPX2 |
| merck2-BC006325_x_at | GTSE1 TRMU |
| merck-NM_001786_a_at | CDK1 RHOBTB1 |
| merck-ENST00000243201_a_at | HJURP |
| merck-NM_001255_s_at | CDC20 |
| merck-NM_004219_x_at | PTTG1 |
| merck2-BC034607_at | ASPM |
| merck2-BC098582_at | KIF14 |
| merck2-AV714642_at | ANLN |
| merck-NM_018131_at | CEP55 |
| merck-NM_002497_at | NEK2 |
| merck-NM_001067_at | TOP2A |
| merck-NM_018685_at | ANLN |
| merck-BC075828_a_at | GTSE1 |
| merck-NM_031299_at | CDCA3 GNB3 |
| merck2-BC107750_at | CDK1 RHOBTB1 |
| merck-NM_004217_at | AURKB |
| merck2-NM_018410_at | HJURP |
| merck-CR596700_a_at | RRM2 |
| merck-NM_016343_at | CENPF |
| merck-BI868409_a_at | MKI67 |
| merck2-CR936650_at | ANLN |
| merck-BF511624_s_at | BUB1B |
| merck-NM_018101_at | CDCA8 |
| merck-U63743_a_at | KIF2C |
| merck2-NM_145060_a_at | SKA1 |
| merck2-BC001651_at | CDCA8 |
| merck-NM_001211_at | BUB1B |
| merck-NM_012484_at | HMMR |
| merck-NM_014750_at | DLGAP5 |
| merck-NM_018136_s_at | ASPM |
| merck2-NM_031966_at | CCNB1 |
| merck-NM_021953_at | FOXM1 |
| merck2-AL519719_a_at | BIRC5 |
| merck-NM_130398_at | EXO1 |
| merck-NM_014176_at | UBE2T |
| merck-NM_005030_at | PLK1 |
| merck-NM_145060_at | SKA1 |
| merck2-AL517462_s_at | — |
| merck-NM_145697_at | NUF2 |
| merck-NM_016426_at | GTSE1 TRMU |
| merck-NM_153824_a_at | PYCR1 |
| merck2-NM_001168_at | BIRC5 |
| merck2-NM_001039535_a_at | SKA1 |
| merck-NM_017947_at | MOCOS |
| merck-NM_152515_at | CKAP2L |
| merck-ENST00000333706_x_at | BIRC5 |
| merck-NM_003318_at | TTK |
| merck-AK223428_a_at | BIRC5 |
| merck-AK024080_a_at | TOP2A |
| merck-NM_002466_at | MYBL2 |
| merck-NM_005480_at | TROAP |
| merck2-ENST00000370966_a_at | DEPDC1 OTUD7A |
| merck-NM_080668_at | CDCA5 |
| merck-ENST00000335534_s_at | KIF18B |
| merck2-ENST00000372927_at | CENPI |
| merck2-BX349325_at | PRR11 |
| merck-BF308644_s_at | CENPI |
| merck-NM_012310_at | KIF4A GDPD2 |
| merck-NM_018304_s_at | PRR11 |
| merck-NM_001790_at | CDC25C |
| merck-CR602926_s_at | CCNB1 |
| merck2-ENST00000333706_s_at | — |
| merck-NM_002417_at | MKI67 |
| merck2-NM_145061_at | SKA3 |
| merck-NM_182513_at | SPC24 |
| merck-NM_019013_at | FAM64A PITPNM3 |
| merck2-NM_001761_at | CCNF |
| merck2-BT006759_at | KIF2C |
| merck-NM_004237_at | TRIP13 |
| merck-NM_152463_s_at | EME1 |
| merck-NM_014791_at | MELK |
| merck-NM_005192_at | CDKN3 |
| merck-AK055931_a_at | SHCBP1 |
| merck-NM_018234_at | STEAP3 |
| merck-AF331796_a_at | NCAPG |
| merck-NM_152259_s_at | TICRR KIF7 |
| merck-NM_198436_s_at | AURKA |
| merck2-AL832036_at | CKAP2L |
| merck2-AK097710_at | CDC25C |
| merck2-NM_017779_at | DEPDC1 |
| merck2-NM_024745_at | SHCBP1 |
| merck-NM_001813_at | CENPE |
| merck2-BG497357_at | NUF2 |
| merck-NM_199413_at | PRC1 |
| merck-hCT1776373.2_s_at | DEPDC1 OTUD7A |
| merck-BC048988_a_at | SKA3 |
| merck2-DQ892840_a_at | CDC6 |
| merck-NM_018248_at | NEIL3 |
| merck-NM_001237_a_at | CCNA2 EXOSC9 |
| merck-NM_033300_at | LRP8 |

A kidney cancer risk model was built from the training set using a general linear model (from the R package) using the following equation:

Kidney Cancer Risk Score=1.54563−(0.19522*prg1)+(0.06519*prg2) (Formula 7),

where “prg1” is a score calculated from the prognosis genes in Table 22 and “prg2” is a score calculated from prognosis genes in Table 23. These scores are calculated by averaging the log2(intensity) of each probe in the geneset.

The performance of this model was evaluated in reserved validation set of 444 samples. FIG. 14 shows the predicted death rate vs. the actual average (running average of 100 samples as ranked by the prediction score) death rate. As shown in the Figure, the model predicts the average death rate very well.

The detailed information about number of samples, number of deaths, and the death rate in each prediction score bin are summarized in Table 24.

TABLE 24
Average death rate versus prediction score.

| Prediction score | Number of samples | Number of deaths | Rate |
|---|---|---|---|
| <0.2 | 138 | 22 | 0.15942029 |
| 0.2-0.3 | 109 | 22 | 0.201834862 |
| 0.3-0.4 | 56 | 13 | 0.232142857 |
| 0.4-0.5 | 33 | 10 | 0.303030303 |
| 0.5-0.6 | 33 | 16 | 0.484848485 |
| 0.6-0.7 | 29 | 13 | 0.448275862 |
| >0.7 | 46 | 33 | 0.717391304 |

Using a threshold of 0.4, the odds ratio for overall survival was 4.5 (95% CI: 2.9-7.0), Fisher's Exact Test p-value=1.2×10⁻¹¹.

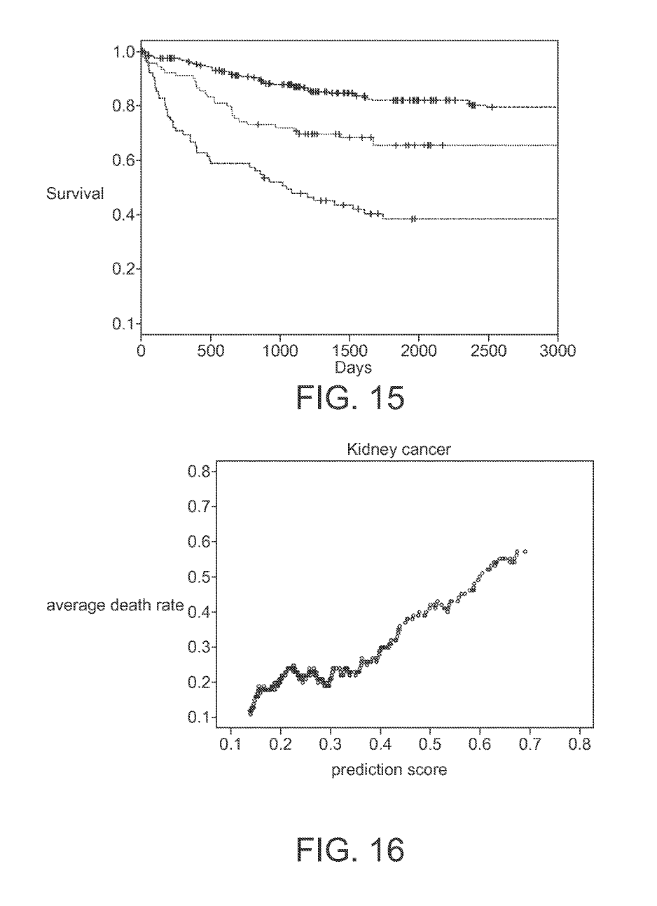

Patients can be further divided into good (risk score<0.35), medium (score 0.35-0.6) and poor (score>0.6) prognosis groups. FIG. 15 shows the Kaplan-Meier curves for these 3 groups. The Chi-square on 2 degrees of freedom is 62.7 (P=2.4×10⁻¹⁴).

The number of genes in each pathway was reduced to 10 genes.

Prognosis Signature Component 1 (prg1):
Probe IDs: merck-NM_021117_at, merck-NM_000901_at, merck2-BC036093_at, merck-AY117034_a_at, merck2-BM977883_at, merck2-NM_020139_at, merck-M13994 a_at, merck2-NM_001608_at, merck-NM_201536_s_at, merck-NM_024563_at
Gene symbols: CRY2, NR3C2, HLF, EMX2OS, FAM221B, BDH2, BCL2, ACADL, NDRG2, NPR3

Prognosis Signature Component 2 (prg2):
Probe IDs: merck-NM_012112_at, merck-NM_004701_at, merck-NM_004217_at, merck-ENST00000243201_a_at, merck-NM_001809_at, merck2-NM_005196_at, merck-NM_145060_at, merck-NM_018131_at, merck-NM_004219 x at, merck-NM_021953_at
Gene symbols: TPX2, CCNB2, AURKB, HJURP, CENPA, CENPF, SKA1, CEP55, PTTG1, FOXM1

The scores derived from these 10-genes correlated to the original scores at the level of 0.97 for prg1 and 0.99 for prg2.

Using the reduced gene sets, the updated predictive model is:

Kidney Cancer Risk Score=0.65473+(−0.10355*prg1)+(0.08053*prg2) (Formula 8).

Note, the exact coefficients will change depending on the final selection of the technology platform (RNAseq vs. arrays, PCR), and the probe sets or gene lists.

FIG. 16 shows the predicted death rate vs. the actual average (running average of 100 samples as ranked by the prediction score) death rate for this updated model. As shown in the Figure, the model predicts the average death rate very well.

The detailed information about number of samples, number of deaths, and the death rate in each prediction score bin are summarized in Table 25.

TABLE 25
Average death rate versus prediction score.

| Prediction score | Number of samples | Number of deaths | Rate |
|---|---|---|---|
| <0.2 | 126 | 20 | 0.158730159 |
| 0.2-0.3 | 121 | 26 | 0.214876033 |
| 0.3-0.4 | 58 | 15 | 0.25862069 |
| 0.4-0.5 | 39 | 11 | 0.282051282 |
| 0.5-0.6 | 28 | 11 | 0.392857143 |
| 0.6-0.7 | 26 | 15 | 0.576923077 |
| >0.7 | 46 | 31 | 0.673913043 |

Using a threshold of 0.42, the odds ratio for overall survival was 4.4 (95% CI: 2.8-6.9), Fisher's Exact Test p-value=4.3×10⁻¹¹.

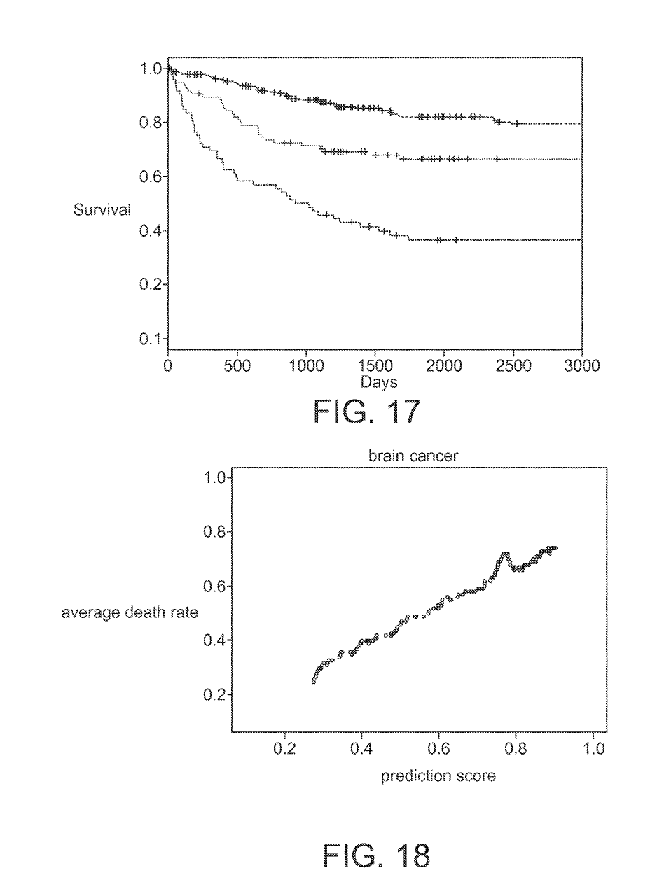

Patients can be further divided into good (risk score<0.35), medium (score 0.35-0.6) and poor (score>0.6) prognosis groups. FIG. 17 shows the Kaplan-Meier curves for these 3 groups. The Chi-square on 2 degrees of freedom is 68.4 (P=1.4×10⁻¹⁵).

### Example 5

### Prognostic Model for Brain Cancer

This example describes a brain cancer prognosis model based on gene expression profiling data. The model contains three gene expression signatures as components. In the second part of the example, the number of genes in each signature is reduced to 10 genes to simplify the implementation of this prognosis model.

A total of 517 samples were profiled by Affymetrix® expression arrays. A composite model was built using the first half of samples and the model validated using the second half of samples. In the first half of samples, 257 samples had outcome data (live or death). In the second half of samples, also 257 had outcome data. The detailed last follow-up dates for the good outcome patients was incomplete. In the first half of samples, 32 out of 95 good outcome patients did not have the last follow-up date. In the second half of samples, 49/121 good outcome patients did not have the last follow-up date. In poor outcome patients, training and validation set each had one without the last follow-up date.

Two groups of genes (100 Affymetrix® probe-sets each) were identified in 257 training samples which were either correlated or anti-correlated with poor outcome. These two groups of genes are displayed in Tables 26 & 27. Genes in Table 27 are highly enriched for cell cycle and cell proliferation pathways.

TABLE 26
Prognosis signature component 1
(anti-correlated with poor outcome) genes

| probe | Gene |
|---|---|
| merck-NM_021117_at | CRY2 |
| merck-NM_152754_at | SEMA3D |
| merck2-NM_001329_at | CTBP2 |
| merck-NM_014912_at | CPEB3 |
| merck-NM_004962_at | GDF10 |
| merck2-BF055210_a_at | CTBP2 |
| merck-ENST00000369884_at | CYP17A1-AS1 |
| merck-NM_002126_at | HLF |
| merck2-BM975249_at | SGMS1 |
| merck-ENST00000344293_s_at | TAF3 |
| merck-AK026683_a_at | SGMS1 |
| merck2-NM_001047160_at | NET1 |
| merck-BM450726_at | ZRANB1 |
| merck2-NM_004657_at | SDPR |
| merck-ENST00000308281_a_at | NET1 |
| merck-NM_001010888_s_at | ZC3H12B |
| merck2-AW591673_at | — |
| merck-BQ709647_a_at | HLF |
| merck-NM_147156_at | SGMS1 |
| merck2-BC036093_at | HLF |
| merck-BC035870_a_at | MIPOL1 |
| merck2-AK125919_at | SCAPER |
| merck2-DB321909_at | SYT15 |
| merck2-BM728590_at | SESN1 |
| merck-NM_173576_s_at | MKX |
| merck-BC016475_a_at | SDPR |
| merck2-BF055210_at | — |
| merck2-BG674122_a_at | HLF |
| merck2-BM555890_a_at | SDPR |
| merck-BC036444_a_at | CPEB3 |
| merck-ENST00000374390_s_at | 8-Mar |
| merck-NM_144591_a_at | C10orf32 |
| merck2-BM728590_a_at | SESN1 |
| merck-ENST00000335753_at | — |
| merck-AK123201_at | MTMR7 VPS37A |
| merck-NM_001609_at | ACADSB |
| merck2-R56002_at | TTC33 |
| merck-NM_019036_s_at | HMGCLL1 |
| merck2-ENST00000379483_at | — |
| merck2-ENST00000308161_at | HMGCLL1 |
| merck-ENST00000368886_at | IKZF5 |
| merck-AK026718_at | SNX2 |
| merck-NM_203441_at | FRA10AC1 |
| merck-NM_138731_at | MIPOL1 |
| merck-NM_031469_at | SH3BGRL2 |
| merck2-AL832477_at | C10orf32 |
| merck-NM_022117_at | TSPYL2 |
| merck-NM_003939_at | BTRC |
| merck2-AL834189_at | VPS37A MTMR7 |
| merck-CR598481_at | TTC33 |
| merck2-DQ269985_at | AKR1C3 |
| merck-AV654599_s_at | AKR1C3 |
| merck2-NM_031912_at | — |
| merck2-CR593590_at | GNAL MPPE1 |
| merck-NM_000997_at | RPL37 |
| merck2-AL136713_a_at | GHITM |
| merck-NM_014454_s_at | SESN1 |
| merck-NM_021785_at | RAI2 |
| merck-NM_017580_a_at | ZRANB1 |
| merck-AK001299_at | VWF |
| merck-ENST00000346874_at | PARD3 |
| merck2-AB188491_at | OTUD1 |
| merck2-Y07511_at | OAT |
| merck-NM_006624_at | ZMYND11 |
| merck-NM_153277_at | SLC22A6 CHRM1 |
| merck2-DA751278_at | RPL13 |
| merck-AK122845_a_at | GABRG1 |
| merck2-BC050310_at | CCNY |
| merck-ENST00000330762_at | NUTM2D |
| merck-AY491432_at | — |
| merck-AK022354_at | METTL10 |
| merck2-NM_130439_at | MXI1 |
| merck-NM_012141_at | INTS6 |
| merck-ENST00000355854_at | CAB39L |
| merck-ENST00000369203_at | SLC18A2 |
| merck-NM_003216_at | TEF |
| merck-BX366291_at | — |
| merck2-W94048_at | TIAL1 |
| merck-NM_024701_at | ASB13 |
| merck-NM_152503_at | MROH8 |
| merck-ENST00000268533_at | NUDT7 |
| merck2-C04536_a_at | MXI1 |
| merck-DA165254_a_at | CACNA2D3 |
| merck-NM_175607_at | CNTN4 |
| merck-AW959468_s_at | — |
| merck2-AI003348_at | NMNAT2 |
| merck-NM_022039_at | FBXW4 |
| merck2-XM_001127131_at | NUDT7 |
| merck-ENST00000369895_a_at | ARL3 |
| merck2-AI192627_at | PPP3CB |
| merck2-BC035128_a_at | MXI1 |
| merck-NM_032138_at | KBTBD7 |
| merck-ENST00000369619_a_at | MXI1 |
| merck-NM_016929_at | CLIC5 |
| merck-ENST00000298035_at | OTUD1 |
| merck-NM_021132_at | PPP3CB |
| merck-CB048235_at | — |
| merck2-AA815447_at | CACNA2D3 |
| merck2-BF248252_at | — |
| merck-NM_001050_at | SSTR2 |

TABLE 27
Prognosis signature component 2
(correlated with poor outcome) genes

| probe | Gene |
|---|---|
| merck-CR596700_a_at | RRM2 |
| merck2-AL517462_s_at | — |
| merck-NM_145060_at | SKA1 |
| merck-NM_198436_s_at | AURKA |
| merck2-NM_001039535_a_at | SKA1 |
| merck2-NM_145060_a_at | SKA1 |
| merck-ENST00000333706_x_at | BIRC5 |
| merck-AK223428_a_at | BIRC5 |
| merck-NM_004219_x_at | PTTG1 |
| merck-NM_012310_at | KIF4A GDPD2 |
| merck-NM_001809_at | CENPA |
| merck2-ENST00000333706_s_at | — |
| merck-NM_001276_at | CHI3L1 |
| merck-NM_018101_at | CDCA8 |
| merck-ENST00000360566_at | RRM2 |
| merck2-BC001651_at | CDCA8 |
| merck2-AF098158_at | TPX2 |
| merck-NM_012112_at | TPX2 |
| merck-NM_005733_at | KIF20A CDC23 |
| merck-U63743_a_at | KIF2C |
| merck2-AK123247_at | MYH11 NDE1 |
| merck2-ENST00000331944_s_at | — |
| merck-NM_181802_at | UBE2C |
| merck2-NM_018410_at | HJURP |
| merck2-BT006759_at | KIF2C |
| merck2-M87338_at | RFC2 |
| merck-NM_152637_at | METTL7B ITGA7 |
| merck-NM_182513_at | SPC24 |
| merck-NM_018154_at | ASF1B PRKACA |
| merck2-AL519719_a_at | BIRC5 |
| merck2-BC007417_at | POC1A |
| merck-NM_021953_at | FOXM1 |
| merck-NM_016426_at | GTSE1 TRMU |
| merck-CR602926_s_at | CCNB1 |
| merck-NM_014791_at | MELK |
| merck-NM_006342_at | TACC3 |
| merck-NM_004701_at | CCNB2 |
| merck-NM_004217_at | AURKB |
| merck-NM_144569_s_at | SPOCD1 |
| merck2-NM_001168_at | BIRC5 |
| merck2-BC006325_at | GTSE1 TRMU |
| merck-NM_018131_at | CEP55 |
| merck-AY605064_at | CLSPN |
| merck-NM_004336_at | BUB1 RGPD6 |
| merck-NM_031299_at | CDCA3 GNB3 |
| merck2-AF043294_at | BUB1 RGPD6 |
| merck2-NM_014397_at | NEK6 |
| merck-NM_001255_s_at | CDC20 |
| merck2-ENST00000370966_a_at | DEPDC1 OTUD7A |
| merck-ENST00000243201_a_at | HJURP |
| merck-NM_003258_at | TK1 |
| merck-CR602847_a_at | KIAA0101 |
| merck-NM_006547_at | IGF2BP3 AMOTL1 MALSU1 |
| merck2-BC006325_x_at | GTSE1 TRMU |
| merck-BC075828_a_at | GTSE1 |
| merck-NM_014750_at | DLGAP5 |
| merck-NM_203394_at | E2F7 |
| merck-ENST00000308604_s_at | LINC00152 MIR4435-1HG |
| merck-AF469667_a_at | MLF1IP |
| merck-BI868409_a_at | MKI67 |
| merck-NM_016639_at | TNFRSF12A CLDN9 |
| merck-CR607300_a_at | MKI67 |
| merck-NM_001237_a_at | CCNA2 EXOSC9 |
| merck-NM_152515_at | CKAP2L |
| merck-AK055931_a_at | SHCBP1 |
| merck-NM_005192_at | CDKN3 |
| merck2-AK000490_a_at | DEPDC1 |
| merck-NM_012291_at | ESPL1 PFDN5 |
| merck-BC106033_s_at | SMC4 |
| merck2-BC034607_at | ASPM |
| merck-NM_152562_s_at | CDCA2 |
| merck-NM_004237_at | TRIP13 |
| merck2-AK026140_at | — |
| merck-NM_001813_at | CENPE |
| merck2-BC005978_at | KPNA2 |
| merck2-NM_024745_at | SHCBP1 |
| merck-CR610123_a_at | POC1A |
| merck-NM_001790_at | CDC25C |
| merck2-Y00472_a_at | SOD2 |
| merck2-BC025232_at | CDC6 |
| merck2-NM_017779_at | DEPDC1 |
| merck-NM_004526_at | MCM2 |
| merck2-BC107750_at | CDK1 RHOBTB1 |
| merck-BX649059_at | GAS2L3 |
| merck-NM_005480_at | TROAP |
| merck-NM_007243_a_at | NRM |
| merck2-NM_031966_at | CCNB1 |
| merck-NM_001024466_s_at | SOD2 |
| merck2-BC005978_s_at | KPNA2 |
| merck-NM_080668_at | CDCA5 |
| merck-NM_004911_at | PDIA4 |
| merck-BC004202_a_at | CHEK1 |
| merck-NM_003504_at | CDC45 |
| merck2-BC098582_at | KIF14 |
| merck2-M36693_a_at | SOD2 |
| merck-NM_012145_a_at | DTYMK |
| merck-NM_017581_at | CHRNA9 |
| merck2-BM464374_at | CENPE |
| merck-NM_001845_at | COL4A1 |
| merck2-DQ890621_at | CDC45 |

TABLE 28
Hypoxia signature

| probe | Gene |
|---|---|
| merck-NM_002627_at | PFKP PITRM1 |
| merck-NM_000302_at | PLOD1 |
| merck-NM_001216_at | CA9 RMRP |
| merck-ENST00000377093_at | KIF1B |
| merck-BC004202_a_at | CHEK1 |
| merck-NM_030949_at | PPP1R14C |
| merck-CR593119_a_at | CLIC4 |
| merck-NM_001255_s_at | CDC20 |
| merck-BG679113_s_at | KRT6A KRT6B KRT6C |
| merck-NM_002421_at | MMP1 |
| merck-BQ217236_a_at | SERPINB5 |
| merck-NM_001793_at | CDH3 |
| merck-NM_001238_at | CCNE1 |
| merck-BU597348_s_at | SYNCRIP |
| merck-NM_006516_at | SLC2A1 |
| merck-BX648425_a_at | DSC2 |
| merck-X15014_a_at | RALA |
| merck-NM_018685_at | ANLN |
| merck-CR614206_a_at | ERO1L |
| merck-NM_001124_at | ADM |
| merck-NM_015440_at | MTHFD1L |
| merck-ENST00000367307_a_at | MTHFD1L |
| merck-NM_058179_at | PSAT1 |
| merck-NM_031415_s_at | GSDMC |
| merck-NM_005557_x_at | KRT16 |
| merck-NM_053016_at | PALM2 PALM2-AKAP2 |
| merck-CR602579_a_at | CTPS1 |
| merck-NM_001428_s_at | ENO1 |
| merck-ENST00000305850_at | CENPN CMC2 |
| merck-NM_005978_at | S100A2 |
| merck-NM_018643_at | TREM1 |
| merck-NM_006505_at | PVR |
| merck-NM_080655_s_at | MSANTD3 |
| merck-NM_001012507_at | CENPW |
| merck-ENST00000258005_a_at | NHSL1 |
| merck-AK129763_at | LINC00673 |
| merck-XM_927868_s_at | PGK1 |
| merck-XM_928117_x_at | FAM106B |
| merck-AL359337_at | ADM |
| merck-AA148856_s_at | SYNCRIP |
| merck2-AI989728_at | SERPINB5 |
| merck2-DQ892208_at | CA9 RMRP |
| merck2-AK022036_at | WWTR1 |
| merck2-AA677426_at | — |
| merck2-AA677426_s_at | — |
| merck2-BC004856_at | NCS1 |
| merck2-BG252150_at | PFKP |
| merck2-BC007633_at | AGO2 |
| merck2-BG400371_at | — |
| merck2-DQ891441_at | — |
| merck2-NM_017522_AS_at | LRP8 |
| merck2-AF039652_at | RNASEH1 |
| merck2-AV714642_at | ANLN |
| merck2-AB030656_at | CORO1C |
| merck2-NM_000291_at | PGK1 |
| merck2-NM_005554_at | KRT6A |
| merck2-BC002829_at | S100A2 |
| merck2-BU681245_at | — |
| merck2-AK225899_a_at | CTPS1 |
| merck2-BC062635_a_at | XPO5 |
| merck2-AF257659_a_at | CALU |
| merck2-CA308717_at | — |
| merck2-X56807_at | DSC2 |
| merck2-CR936650_at | ANLN |
| merck2-AY423725_a_at | PGK1 |
| merck2-BC103752_a_at | PGK1 |

The prognosis model was built in the training set using a general linear model (from the R package) using the following equation:

Brain Cancer Risk Score=−0.28894+(−0.12713*prg1)+(0.09353*prg2)+(0.15399*hscore) (Formula 9),

where “prg1” is a score calculated from prognosis genes in Table 26, “prg2” is a score calculated from prognosis genes in Table 27, and “hscore” is a hypoxia pathway score calculated from genes in Table 28. The scores can be calculated by averaging the log2(intensity) of each probe in the geneset.

The performance of this model was evaluated in reserved validation set of 257 samples. FIG. 18 shows the predicted death rate vs. the actual average (running average of 100 samples as ranked by the prediction score) death rate. As shown in the Figure, the model predicts the average death rate very well.

The detailed information about number of samples, number of deaths, and the death rate in each prediction score bin are summarized in Table 29.

TABLE 29
Average death rate versus prediction score.

| Prediction score | Number of samples | Number of deaths | Rate |
|---|---|---|---|
| <0.3 | 57 | 9 | 0.157894737 |
| 0.3-0.5 | 35 | 14 | 0.4 |
| 0.5-0.7 | 30 | 17 | 0.566666667 |
| 0.7-0.9 | 83 | 58 | 0.698795181 |
| >0.9 | 52 | 38 | 0.730769231 |

Using a threshold of 0.58, the odds ratio for overall survival was 6.3 (95% CI: 3.6-10.9), Fisher's Exact Test p-value=1.5×10⁻¹¹.

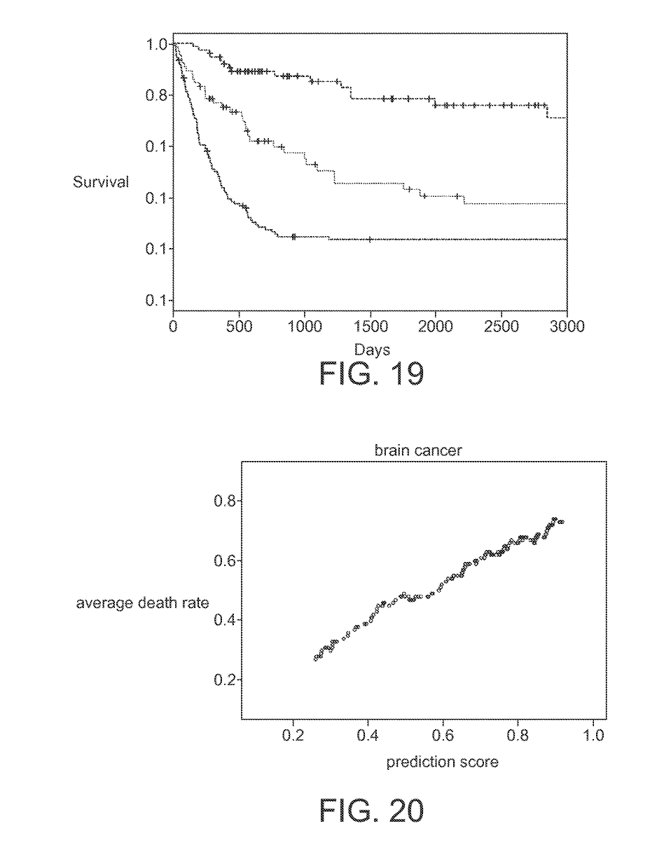

Patients can be further divided into good (risk score<0.4), medium (score 0.4-0.75) and poor (score>0.75) prognosis groups. FIG. 19 shows the Kaplan-Meier curves for these 3 groups. The Chi-square on 2 degrees of freedom is 57.5 (P=3.2×10⁻¹³).

The number of genes in each pathway was reduced to 10 genes.

Prognosis Signature Component 1 (prg1):
Probe IDs: merck-NM_002126_at, merck2-BF055210_a_at, merck-NM_014912_at, merck2-BM975249_at, merck2-NM_001329_at, merck-BM450726_at, merck-NM_003939_at, merck-NM_001609_at, merck-NM_001010888_s_at, merck-ENST00000380064 at
Gene symbols: HLF, CTBP2, CPEB3, SGMS1, CTBP2, ZRANB1, BTRC, ACADSB, ZC3H12B, REPS2

Prognosis Signature Component 2 (prg2):
Probe IDs: merck-NM_145060_at, merck-NM_012112_at, merck-NM_004701_at, merck-NM_001809_at, merck-ENST00000333706_x_at, merck-CR596700_a_at, merck-NM_198436_s_at, merck-NM_004217_at, merck-U63743_a_at, merck2-BC001651_at
Gene symbols: SKA1, TPX2, CCNB2, CENPA, BIRC5, RRM2, AURKA, AURKB, KIF2C, CDCA8

Hypoxia Signature:
Probe IDs: merck-NM_018643_at, merck-BC010860_a_at, merck-NM_013332_at, merck-X15014_a_at, merck-NM_001625_a_at, merck-NM_001024466_s_at, merck2-BQ015108_at, merck2-BC103752_a_at, merck-NM_001039667_s_at, merck2-NM_001042422_at
Gene symbols: TREM1, SERPINE1, HILPDA, RALA, AK2, SOD2, ARL4C, PGK1, ANGPTL4, SLC16A3

The scores derived from these 10-genes are correlated to the original scores at the level of 0.97 for prgl, 0.98 for prg2 and 0.84 for the hypoxia signature.

Using the reduced gene sets, the updated predictive model is:

Brain Cancer Risk Score=−1.320607+(−0.003094*prg1)+(0.094341*prg2)+(0.143865*hscore) (Formula 10).

Note, the exact coefficients will change depending on the final selection of the technology platform (RNAseq vs. arrays, PCR), and the probe sets or gene lists.

FIG. 20 shows the predicted death rate vs. the actual average (running average of 100 samples as ranked by the prediction score) death rate for this updated model. As shown in the Figure, the model predicts the average death rate very well.

The detailed information about number of samples, number of deaths, and the death rate in each prediction score bin are summarized in Table 30.

TABLE 30
Average death rate versus prediction score.

| Prediction score | Number of samples | Number of deaths | Rate |
|---|---|---|---|
| <0.3 | 59 | 11 | 0.186440678 |
| 0.3-0.5 | 32 | 12 | 0.375 |
| 0.5-0.7 | 40 | 24 | 0.6 |
| 0.7-0.9 | 73 | 46 | 0.630136986 |
| >0.9 | 53 | 43 | 0.811320755 |

Using a threshold of 0.6, the odds ratio for overall survival is 5.7 (95% CI: 3.3-9.9), Fisher's Exact Test p-value=6.7×10⁻¹¹.

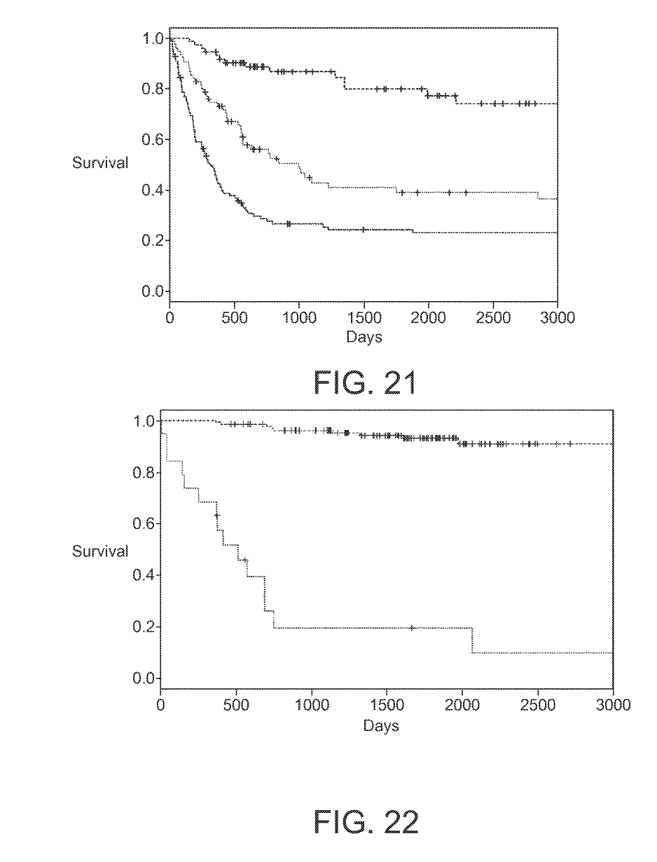

Patients can be further divided into good (risk score<0.4), medium (score 0.4-0.75) and poor (score>0.75) prognosis groups. FIG. 21 shows the Kaplan-Meier curves for these 3 groups. The Chi-square on 2 degrees of freedom is 56.0 (P=6.8×10⁻¹³).

### Example 6

### Prognostic Model for Prostate Cancer

This example describes a prostate cancer prognosis model based on gene expression profiling data. The model contains two gene expression signatures as components. In the second part of the example, the number of genes in each signature was reduced to 10 genes to simplify the implementation of this prognosis model.

A total of 302 samples were profiled by Affymetrix® expression arrays. A composite model was built using the first half of samples and the model validated in the second half of samples. In the first half of samples, 151 samples had outcome data (live or death). In the second half of samples, 151 samples had outcome data. The detailed last follow-up dates for the good outcome patients are incomplete. In the first half of samples, 16 out of 137 good outcome patients did not have the last follow-up date. In the second half of samples, 16/127 good outcome patients did not have the last follow-up date. In poor outcome patients, all but one had last follow-up dates.

Two groups of genes (100 Affymetrix® probe-sets each) were identified in 151 training samples which were either correlated or anti-correlated with poor outcome. These two groups of genes are displayed in Tables 31 & 32. Genes in Table 32 are highly enriched for cell cycle and cell proliferation pathways.

The model was built in the training set using a general linear model (from the R package) using the following equation:

Prostate Cancer Risk Score=0.41973 +0.08610*(prg2−prg1) (Formula 11),

where “prg1” is a score calculated from prognosis genes in Table 31 and “prg2” is a score calculated from prognosis genes in Table 32. Scores can be calcualted by averaging the log2(intensity) of each probe in the geneset.

The performance of this model is evaluated in reserved validation set of 151 samples.

Using a threshold of 0.4, the odds ratio for overall survival was 51.4 (95% CI: 14.1-186.9), Fisher's Exact Test p-value =2.2×10⁻¹¹.

The Kaplan-Meier curves using the same threshold are shown in FIG. 22. The Chi-square on 1 degrees of freedom is 123 (P=0).

The number of genes in each pathway was reduced to 10 genes.

Prognosis Signature Component 1 (prg1):
Probe IDs: merck-NM_012134_at, merck-NM_021965_s_at, merck-BC064695_s_at, merck2-BF681326_at, merck2-NM_015385_at, merck-NM_032105_at, merck-AF055081_s_at, merck-NM_001299_at, merck2-AI745408_a_at, merck-CA438563_at
Gene symbols: LMOD1, PGM5, MYLK, SYNPO2, SORBS1, PPP1R12B, DES, CNN1, MYH11, MYOCD

Prognosis Signature Component 2 (prg2):
Probe IDs: merck-NM_012112_at, merck-NM_181802_at, merck-NM_004219_x_at, merck2-AK023483_at, merck-NM_001809_at, merck-NM_198436_s_at, merck-NM_080668_at, merck-NM_018454_at, merck-NM_004217_at, merck-ENST00000333706_x_at
Gene symbols: TPX2, UBE2C, PTTG1, NUSAP1, CENPA, AURKA, CDCA5, NUSAP1, AURKB, BIRC5,

The scores derived from these 10-genes correlated to the original scores at the level of 0.98 for both prg1 and prg2.

Using the reduced gene sets, the updated predictive model is:

Prosate Cancer Risk Score=0.34044+0.06186*(prg2−prg1) (Formula 12).

Note, the exact coefficients will change depending on the final selection of the technology platform (RNAseq vs. arrays, PCR), and the probe sets or gene lists.

The performance of the reduced genesets was the same as the original genesets. Using a threshold of 0.4, the odds ratio for overall survival is 51.4 (95% CI: 14.1-186.9), Fisher's Exact Test p-value=2.2×10⁻¹¹.

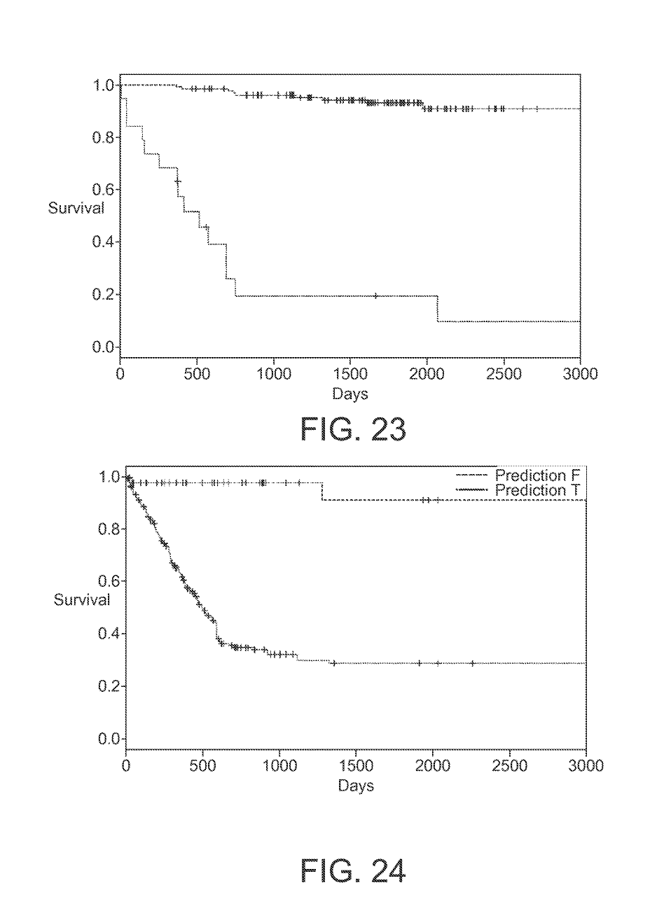

The Kaplan-Meier curves using the same threshold are shown in FIG. 23. The Chi-square on 1 degrees of freedom is 123 (P=0).

TABLE 31
Prognosis signature component 1
(anti-correlated with poor outcome)

| probe | Gene |
|---|---|
| merck-NM_021965_s_at | PGM5 |
| merck-BC064695_s_at | MYLK |
| merck2-NM_152795_at | HIF3A PPP5C |
| merck2-BU195365_at | LMOD1 |
| merck-NM_005197_s_at | FOXN3 |
| merck-NM_032801_at | JAM3 |
| merck2-BC036093_at | HLF |
| merck-ENST00000343365_a_at | LMOD1 |
| merck-AL832580_at | RNF180 |
| merck2-BX118828_at | — |
| merck-NM_001025266_at | C3orf70 |
| merck2-AW964876_at | FOXN3 |
| merck-NM_004078_at | CSRP1 |
| merck-J02854_at | MYL9 |
| merck2-AI598275_at | CSRP1 |
| merck-AK098218_a_at | PGM5-AS1 |
| merck-BQ709647_a_at | HLF |
| merck-NM_213674_x_at | TPM2 RMRP |
| merck-NM_181526_s_at | MYL9 |
| merck-NM_014365_at | HSPB8 |
| merck-AK093957_s_at | MIR143HG |
| merck2-BX350133_at | — |
| merck-NM_033303_at | ADRA1A |
| merck-NM_003462_at | DNALI1 |
| merck-NM_002126_at | HLF |
| merck-NM_007177_at | FAM107A |
| merck-NM_012134_at | LMOD1 |
| merck2-CD557691_at | NFIA |
| merck-ENST00000371189_s_at | NFIA |
| merck-ENST00000372045_at | CHRDL1 |
| merck2-BG674122_a_at | HLF |
| merck2-EB387139_a_at | ATP1A2 |
| merck2-AI692523_at | — |
| merck-NM_001042_at | SLC2A4 |
| merck2-BF681326_at | SYNPO2 |
| merck-NM_013377_at | PDZRN4 |
| merck-NM_000898_at | MAOB MAOA |
| merck-ENST00000261302_a_at | FOXN3 |
| merck2-NM_022844_s_at | — |
| merck-BC107758_at | TNS1 |
| merck-NM_004137_at | KCNMB1 KCNIP1 LOC101928033 |
| merck2-NM_015385_at | SORBS1 |
| merck-D10667_a_at | MYH11 NDE1 |
| merck2-AL532587_at | TPM2 RMRP |
| merck2-BC107783_s_at | — |
| merck-BX381493_s_at | ANKRD35 |
| merck-AL833294_s_at | SYNPO2 |
| merck2-NM_000195_at | HPS1 |
| merck2-AL831991_at | ATP1A2 |
| merck2-NM_003734_at | AOC3 |
| merck2-DC364710_x_at | NEXN |
| merck-ENST00000361490_a_at | HPS1 |
| merck-ENST00000330010_a_at | NEXN |
| merck-NM_004975_at | KCNB1 |
| merck-NM_000961_at | PTGIS |
| merck-NM_003734_at | AOC3 |
| merck2-AI745408_a_at | MYH11 |
| merck2-NM_147162_at | IL11RA |
| merck2-BC113456_at | MYLK |
| merck2-H40930_at | NECAB1 |
| merck-NM_053029_s_at | MYLK |
| merck2-CD299407_x_at | NEXN |
| merck2-EB387733_a_at | SORBS1 |
| merck-BQ888844_a_at | SORBS1 |
| merck-ENST00000312358_s_at | SPEG |
| merck-AI918006_at | UBXN10 |
| merck-NM_002398_at | MEIS1 |
| merck-NM_198995_s_at | CCDC178 |
| merck2-NM_033254_at | — |
| merck-BU681386_at | SCN7A |
| merck2-CD299407_at | NEXN |
| merck-NM_001299_at | CNN1 |
| merck-NM_025220_s_at | ADAM33 |
| merck-NM_203441_at | FRA10AC1 |
| merck2-BX464303_at | GSTM3 |
| merck2-ENST00000371953_at | PTEN |
| merck-NM_020899_s_at | ZBTB4 |
| merck2-H40930_x_at | NECAB1 |
| merck-NM_001456_s_at | FLNA |
| merck2-NM_001037954_at | DIXDC1 |
| merck-AK024986_at | PTEN |
| merck2-AL554563_at | ACTA2 |
| merck-NM_022062_s_at | PKNOX2 |
| merck-AY358229_a_at | MSRB3 |
| merck-NM_001387_at | DPYSL3 |
| merck2-BC034387_at | SLC2A4 |
| merck2-AA536214_at | — |
| merck-NM_020925_s_at | CACHD1 |
| merck-AK056079_s_at | JAM2 GABPA |
| merck-AL833622_a_at | MSRB3 |
| merck-NM_001083_at | PDE5A |
| merck2-BC055084_at | NEXN |
| merck2-NM_016826_at | OGG1 CAMK1 |
| merck-NM_001759_at | CCND2 |
| merck-NM_014057_a_at | OGN |
| merck-AK026168_at | — |
| merck2-AI288607_at | — |
| merck-NM_145728_at | SYNM |
| merck2-AK056845_at | — |
| merck-NM_002725_at | PRELP OPTC |

TABLE 32
Prognosis signature component 2
(correlated with poor outcome)

| probe | Gene |
|---|---|
| merck2-AF225416_at | SPC25 |
| merck-NM_020675_at | SPC25 |
| merck-BC003664_a_at | KIF4A |
| merck2-NM_024037_at | AUNIP |
| merck-NM_001809_at | CENPA |
| merck-NM_181802_at | UBE2C |
| merck-NM_014176_at | UBE2T |
| merck-NM_005733_at | KIF20A CDC23 |
| merck-NM_013277_a_at | RACGAP1 |
| merck-CR602847_a_at | KIAA0101 |
| merck2-DQ890621_at | CDC45 |
| merck-NM_018248_at | NEIL3 |
| merck-BC035392_at | HMMR |
| merck2-NM_005196_at | CENPF |
| merck-NM_004219_x_at | PTTG1 |
| merck2-AK097710_at | CDC25C |
| merck-NM_001786_a_at | CDK1 RHOBTB1 |
| merck-NM_144508_at | CASC5 |
| merck-NM_016343_at | CENPF |
| merck-DA823877_a_at | CDK1 RHOBTB1 |
| merck-NM_152259_s_at | TICRR KIF7 |
| merck-NM_004701_at | CCNB2 |
| merck-NM_003504_at | CDC45 |
| merck-AK055176_s_at | FANCI |
| merck-BC075828_a_at | GTSE1 |
| merck-NM_203394_at | E2F7 |
| merck-NM_001039841_s_at | ARHGAP11A ARHGAP11B |
| merck-NM_001790_at | CDC25C |
| merck-NM_004217_at | AURKB |
| merck-NM_002497_at | NEK2 |
| merck-ENST00000246083_s_at | DNAJC9 ZFYVE26 |
| merck2-AB046790_at | CASC5 |
| merck-NM_031299_at | CDCA3 GNB3 |
| merck-BC048988_a_at | SKA3 |
| merck-NM_016426_at | GTSE1 TRMU |
| merck-NM_014750_at | DLGAP5 |
| merck-NM_021953_at | FOXM1 |
| merck2-BC107750_at | CDK1 RHOBTB1 |
| merck-NM_014791_at | MELK |
| merck-NM_002466_at | MYBL2 |
| merck-NM_001067_at | TOP2A |
| merck2-NM_203399_at | STMN1 |
| merck-NM_130398_at | EXO1 |
| merck-NM_006461_at | SPAG5 |
| merck2-BX091454_a_at | RACGAP1 |
| merck2-BE856617_at | AURKA |
| merck-NM_080668_at | CDCA5 |
| merck-AK093235_s_at | TDP1 |
| merck2-AF043294_at | BUB1 RGPD6 |
| merck2-DB485269_a_at | — |
| merck-NM_018101_at | CDCA8 |
| merck-BC024211_a_at | NCAPH |
| merck-NM_012310_at | KIF4A GDPD2 |
| merck-NM_018136_s_at | ASPM |
| merck-BF511624_s_at | BUB1B |
| merck-NM_012112_at | TPX2 |
| merck2-ENST00000372927_at | CENPI |
| merck2-BC006325_x_at | GTSE1 TRMU |
| merck-AK129748_s_at | STMN1 |
| merck-BF308644_s_at | CENPI |
| merck-NM_174942_a_at | GAS2L3 |
| merck-NM_198436_s_at | AURKA |
| merck-NM_002417_at | MKI67 |
| merck-NM_001255_s_at | CDC20 |
| merck2-AK025810_at | WDR5 |
| merck-NM_003258_at | TK1 |
| merck2-DQ892840_a_at | CDC6 |
| merck-NM_003201_at | TFAM |
| merck-NM_017669_at | ERCC6L |
| merck2-BC014353_a_at | STMN1 |
| merck-CR622584_s_at | CHEK2 |
| merck-NM_004336_at | BUB1 RGPD6 |
| merck2-AL517462_s_at | — |
| merck-AK057037_at | FEZF1-AS1 |
| merck2-AL703195_s_at | — |
| merck-NM_001002876_at | CENPM |
| merck-NM_004203_a_at | PKMYT1 |
| merck2-XM_937756_a_at | FEN1 |
| merck-ENST00000243201_a_at | HJURP |
| merck-ENST00000373940_a_at | ZWINT |
| merck-AI418253_at | PMS2LP2 |
| merck-BI868409_a_at | MKI67 |
| merck2-ENST00000373899_at | TFAM |
| merck-NM_020394_at | ZNF695 ZNF670-ZNF695 |
| merck-BQ653044_a_at | EZH2 |
| merck-CR602926_s_at | CCNB1 |
| merck2-NM_018944_at | MIS18A |
| merck-NM_032117_at | MND1 |
| merck-NM_018454_at | NUSAP1 |
| merck-NM_005192_at | CDKN3 |
| merck-BC038772_s_at | MCM4 |
| merck2-BT006759_at | KIF2C |
| merck-CR596700_a_at | RRM2 |
| merck2-BC106011_a_at | ACP1 |
| merck2-AK023483_at | NUSAP1 |
| merck-NM_003533_at | HIST1H3I |
| merck2-BC022400_at | METTL6 |
| merck2-BC034607_at | ASPM |
| merck2-NM_031966_at | CCNB1 |
| merck-NM_138419_s_at | MTFR2 |

### Example 7

### Prognostic Model for Pancreatic Cancer

This example describes a pancreatic cancer prognosis model based on gene expression profiling data. The model contains two gene expression signatures as components. In the second part of the example, the number of genes in each signature is reduced to 10 genes to simplify the implementation of this prognosis model.

A total of 525 samples were profiled by Affymetrix® expression arrays. A composite model was built using the first half of samples and the model validated using the second half of samples. In the first half of samples, 261 samples had outcome data (live or death). In the second half of samples, also 263 samples had outcome data. The detailed last follow-up dates for the good outcome patients are incomplete. In the first half of samples, 12 out of 97 good outcome patients did not have the last follow-up date. In the second half of samples, 30/136 good outcome patients did not have the last follow-up date.

Two groups of genes (100 Affymetrix® probe-sets each) were identified in 261 training samples which are either correlated or anti-correlated with poor outcome. These two groups of genes are displayed in Tables 33 & 34. Genes in Table 34 are highly enriched for cell cycle and cell proliferation pathways.

A model was built in the training set using a general linear model (from the R package) using the following equation:

Pancreatic Cancer Risk Score=Risk Score=0.467962 +0.076686*(prg2−prg1) (Formula 13),

where “prg1” is a score calculated from prognosis genes in Table 33 and “prg2” is a score calculated from prognosis genes in Table 34. The scores can be calculated by averaging the log2(intensity) of each probe in the geneset.

The performance of this model is evaluated in reserved validation set of 263 samples.

Using a threshold of 0.5, the odds ratio for overall survival was 35.2 (95% CI:6 8.3-148), Fisher's Exact Test p-value=3.7×10⁻¹⁴.

The Kaplan-Meier curves using the same threshold is shown in FIG. 24. The Chi-square on 1 degrees of freedom is 33.9 (P=5.82×10⁻⁹).

The number of genes in each pathway was reduced to 10 genes.

Prognosis Signature Component 1 (prg1):
Probe IDs: merck2-AL133657_at, merck2-NM_033026_at, merck-NM_018711_at, merck-BC001946_a_at, merck-NM_006650_at, merck-BI552493_a_at, merck-ENST00000371069_a_at, merck-NM_004644_at, merck-BC045704 a_at,merck2-NM_005374_at
Gene symbols: RUNDC3A, PCLO, SVOP, CELF4, CPLX2, SCG3, DNAJC6, AP3B2, SCN3B, MPP2

Prognosis Signature Component 2 (prg2):
Probe IDs: merck-NM_006142_at, merck-NM_000228_at, merck2-NM_183247_a_at, merck-NM_016445_at, merck-NM_002447_at, merck-NM_{—}024009_at merck-NM_080388 at merck-NM_003979 at merck-NM_001005376 at merck-NM_001747_at
Gene symbols: SFN, LAMB3, TMPRSS4, PLEK2, MST1R, GJB3, S100A16, GPRC5A, PLAUR, CAPG

The scores derived from these 10-genes correlated to the original scores at the level of 0.97 for prg1 and 0.98 for prg2.

Using the reduced gene sets, the updated predictive model is:

Pancreatic Cancer Risk Score=Risk Score=0.504576+0.049284*(prg2−prg1) (Formula 14).

Note, the exact coefficients will change depending on the final selection of the technology platform (RNAseq vs. arrays, PCR), and the probe sets or gene lists.

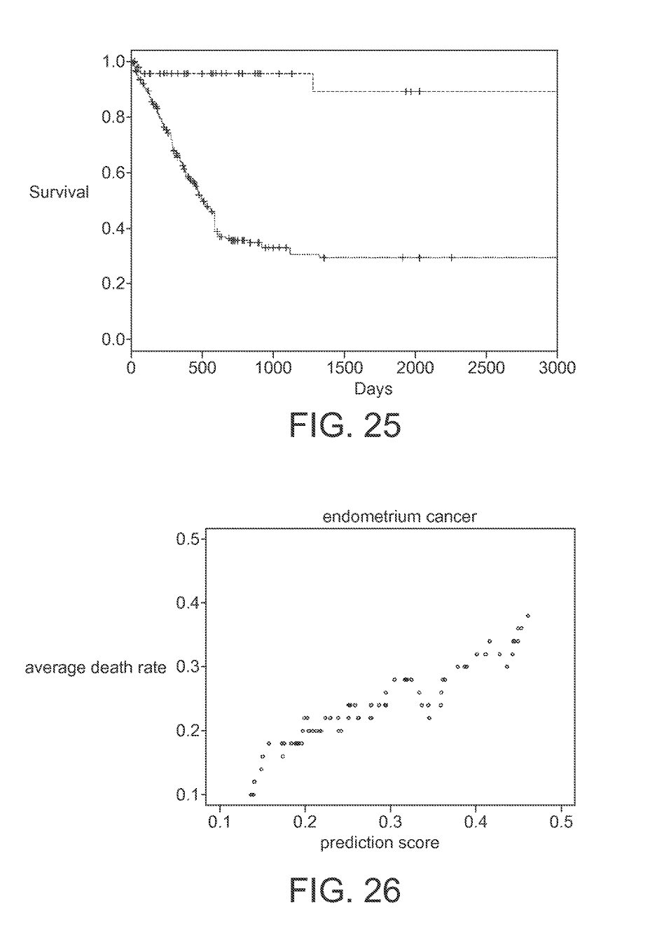

The performance of the reduced genesets is similar the original genesets. Using a threshold of 0.5, the odds ratio for overall survival is 22.5 (95% CI: 6.8-74.7), Fisher's Exact Test p-value=8.4×10⁻¹³. The Kaplan-Meier curves using the same threshold are shown in FIG. 25. The Chi-square on 1 degrees of freedom is 30.2 (P=3.8×10⁻⁸).

TABLE 33
Prognosis signature component 1
(anti-correlated with poor outcome)

| probe | Gene |
|---|---|
| merck-NM_024557_at | RIC3 |
| merck-NM_171998_at | RAB39B |
| merck-ENST00000379272_at | ACSL6 |
| merck-XM_938173_at | CELF4 |
| merck-NM_024026_x_at | MRP63 |
| merck-BC001946_a_at | CELF4 |
| merck2-BX647514_a_at | RIC3 |
| merck2-NM_020180_at | CELF4 |
| merck2-DB523436_at | ACSL6 |
| merck-AK056249_at | — |
| merck2-AL832601_at | RIC3 TUB |
| merck-NM_144576_at | COQ10A |
| merck-NM_020818_at | UNC79 |
| merck2-AL133657_at | RUNDC3A |
| merck-AK075495_at | NDFIP1 |
| merck-NM_030802_at | FAM117A |
| merck-BC044777_at | TMX4 |
| merck-NM_006695_a_at | RUNDC3A |
| merck-NM_032829_at | FAM222A |
| merck2-AL532654_at | CIRBP |
| merck-AK125327_a_at | UNC79 |
| merck-BG212691_s_at | EPM2A |
| merck-ENST00000377770_a_at | DPP6 |
| merck2-NM_138362_at | FAM104B |
| merck-CR605402_at | TBCK |
| merck2-AF546872_at | PACRG |
| merck-NM_020708_at | SLC12A5 |
| merck-AW297465_at | — |
| merck2-BI761148_a_at | CIRBP |
| merck2-AK092094_at | SLC25A5-AS1 SLC25A5 |
| merck-NM_152410_at | PACRG |
| merck-BC037882_at | — |
| merck-NM_020949_s_at | SLC7A14 |
| merck-AK055712_at | LOC728705 |
| merck-NM_022151_at | MOAP1 |
| merck-NM_138362_at | FAM104B |
| merck-NM_003179_at | SYP PRICKLE3 |
| merck-NM_021156_a_at | TMX4 |
| merck-NM_006650_at | CPLX2 |
| merck-NM_001033002_s_at | RPAIN |
| merck-NM_170710_at | WDR17 |
| merck2-NM_033026_at | PCLO |
| merck-BU170673_at | — |
| merck-NM_016188_at | ACTL6B TFR2 |
| merck2-BC028357_at | CLGN |
| merck2-AL832187_at | ARMCX5-GPRASP2 |
| | GPRASP2 BHLHB9 |
| merck-NM_001280_a_at | CIRBP |
| merck-BX640845_a_at | FSTL4 |
| merck2-AK094546_at | QDPR |
| merck2-NM_172232_at | ABCA5 |
| merck2-ENST00000379240_at | ACSL6 |
| merck-NM_004362_at | CLGN |
| merck-NM_001039350_at | DPP6 |
| merck-BC035377_at | DMTF1 |
| merck-AF052119_at | SLC25A4 |
| merck2-AK074845_x_at | NUDT9 |
| merck2-AK093871_at | CXXC4 |
| merck-ENST00000332709_at | PGRMC2 |
| merck-BC018917_a_at | MYT1 |
| merck-BC009714_a_at | RAB39B |
| merck-CA868555_a_at | RIC3 |
| merck-NM_007185_at | CELF3 |
| merck-AK094547_at | SLC7A14 |
| merck2-BM977387_at | — |
| merck-ENST00000371069_a_at | DNAJC6 |
| merck-NM_144611_s_at | CYB5D2 |
| merck2-DB479534_at | BEX2 |
| merck2-BY798024_at | UNC80 |
| merck-NM_173092_a_at | KCNH6 DCAF7 |
| merck-AI474150_a_at | ISCA1 |
| merck2-BU687744_at | — |
| merck-NM_152503_at | MROH8 |
| merck2-CK903584_at | SERPINI1 |
| merck-NM_019114_at | EPB41L4B |
| merck-NM_014723_at | SNPH SDCBP2 |
| merck2-CD742622_at | TARBP2 |
| merck-CK819476_s_at | XPNPEP2 |
| merck-AF086195_at | DCUN1D5 |
| merck-NM_145170_at | TTC18 |
| merck2-BC020263_at | CYB5D2 |
| merck2-NM_019589_at | YLPM1 |
| merck2-BF224377_at | — |
| merck-CR596771_a_at | QDPR |
| merck-AK123831_at | CDS2 |
| merck2-BF433548_at | — |
| merck-NM_015063_at | SLC8A2 |
| merck-NM_025212_a_at | CXXC4 LOC101929468 |
| merck-BX537526_at | SLC24A5 |
| merck2-BG695979_at | — |
| merck-AK090762_s_at | — |
| merck2-AL517382_at | AKAP14 |
| merck-AK127804_at | RFX3 LOC101929247 |
| merck-AK123201_at | MTMR7 VPS37A |
| merck-BM681832_at | — |
| merck-AK127501_at | — |
| merck-AK002023_at | CTDP1 |
| merck-NM_033053_s_at | DMRTC1 DMRTC1B |
| merck-AK124803_at | PGBD5 |
| merck2-BF304197_at | — |
| merck-ENST00000372943_at | FITM2 |

TABLE 34
Prognosis signature component 2
(correlated with poor outcome)

| probe | Gene |
|---|---|
| merck-NM_001747_at | CAPG |
| merck-NM_004004_s_at | GJB2 |
| merck2-BC071703_at | GJB2 |
| merck-NM_006142_at | SFN |
| merck2-AF177862_a_at | HN1 |
| merck-NM_000228_at | LAMB3 |
| merck-NM_080388_at | S100A16 |
| merck-NM_007267_at | TMC6 |
| merck2-NM_009587_s_at | — |
| merck-NM_018685_at | ANLN |
| merck2-NM_001048201_at | UHRF1 |
| merck2-NM_001042685_s_at | — |
| merck2-CR936650_at | ANLN |
| merck2-X74039_at | PLAUR |
| merck-NM_001005376_at | PLAUR |
| merck-NM_000213_at | ITGB4 GALK1 |
| merck2-AF491781_a_at | OSBPL3 |
| merck-NM_018131_at | CEP55 |
| merck-BC017731_a_at | OSBPL3 |
| merck-BC105943_s_at | LGALS9 LGALS9B |
| | LGALS9C FAM106B |
| merck2-NM_001042422_at | SLC16A3 |
| merck-NM_003979_at | GPRC5A |
| merck-NM_006681_at | NMU |
| merck2-BM543893_x_at | PLAUR |
| merck-NM_005980_at | S100P |
| merck-X15014_a_at | RALA |
| merck2-AF318350_at | TTYH3 |
| merck2-BG680883_at | — |
| merck-BC046920_a_at | NQO1 |
| merck-CR407664_a_at | PHLDA2 |
| merck-BI868409_a_at | MKI67 |
| merck2-AK223027_at | PHLDA2 |
| merck-BG677853_a_at | LAMC2 |
| merck-NM_005620_at | S100A11 |
| merck2-NM_183247_a_at | TMPRSS4 |
| merck-AF086216_at | SERPINB5 |
| merck-NM_005562_at | LAMC2 |
| merck-NM_145903_s_at | HMGA1 |
| merck2-NM_001005377_at | PLAUR |
| merck2-AK097588_at | ATL3 |
| merck-NM_018715_a_at | RCC2 |
| merck-NM_000189_at | HK2 |
| merck-NM_001005377_s_at | PLAUR |
| merck-NM_019034_at | RHOF TMEM120B |
| merck-AI924527_a_at | TMPRSS4 |
| merck-BC042436_at | — |
| merck-NM_015459_s_at | ATL3 |
| merck-BM806310_a_at | OSBPL3 |
| merck2-BC013892_at | PVRL4 |
| merck-NM_001037330_s_at | TRIM16L TRIM16 |
| merck2-AL517462_s_at | — |
| merck-CR596700_a_at | RRM2 |
| merck-NM_014568_s_at | GALNT5 |
| merck-NM_025250_at | TTYH3 |
| merck2-AI701192_at | LAMC2 |
| merck-NM_002639_at | SERPINB5 |
| merck-NM_004701_at | CCNB2 |
| merck-NM_012112_at | TPX2 |
| merck-NM_001793_at | CDH3 |
| merck2-BG675923_x_at | — |
| merck2-AI701192_x_at | LAMC2 |
| merck2-AV714642_at | ANLN |
| merck-NM_002447_at | MST1R |
| merck-NM_033520_at | C19orf33 YIF1B PPP1R14A |
| merck-NM_014791_at | MELK |
| merck2-M62898_x_at | ANXA2 |
| merck-NM_000422_x_at | KRT17 |
| merck-NM_000445_at | PLEC |
| merck-ENST00000335534_s_at | KIF18B |
| merck-NM_002250_at | KCNN4 |
| merck2-AF098158_at | TPX2 |
| merck-NM_014624_at | S100A6 |
| merck-CR607300_a_at | MKI67 |
| merck-NM_003844_at | TNFRSF10A |
| merck-NM_181802_at | UBE2C |
| merck-NM_002068_at | GNA15 |
| merck-BC001459_s_at | RAD51 |
| merck-NM_005975_at | PTK6 |
| merck-AY358204_a_at | TMEM92 |
| merck2-AF070544_at | SLC2A1 |
| merck2-NM_001083947_at | TMPRSS4 |
| merck-NM_012101_at | TRIM29 |
| merck2-AL831846_at | CELSR1 |
| merck-NM_002417_at | MKI67 |
| merck-AL582254_x_at | — |
| merck2-NM_005975_a_at | — |
| merck2-BT009912_x_at | — |
| merck-AB208913_a_at | ITGB4 |
| merck-NM_014750_at | DLGAP5 |
| merck2-BT009912_at | — |
| merck-NM_003258_at | TK1 |
| merck-NM_024009_at | GJB3 |
| merck-NM_199129_at | TMEM189 |
| merck-NM_016445_at | PLEK2 |
| merck-NM_002306_s_at | LGALS3 |
| merck-NM_021103_a_at | TMSB10 |
| merck-NM_005978_at | S100A2 |
| merck-NM_020672_at | S100A14 |
| merck-ENST00000360566_at | RRM2 |
| merck-NM_025049_at | PIF1 |

### Example 8

### Prognostic Model for Endometrium Cancer

This example describes an endometrium cancer prognosis model based on gene expression profiling data. The model contains two gene expression signatures as components. In the second part of the example, the number of genes in each signature is reduced to 10 genes to simplify the implementation of this prognosis model.

A total of 410 samples were profiled by Affymetrix® expression arrays. A composite model was built using the first half of samples and the model validated using the second half of samples. In the first half of samples, 204 samples had outcome data (alive or dead). Among them, 140 had good outcome and 64 had poor outcome. In the good outcome patients, 12 did not have tumor grade data, and in the poor outcome patients, 17 did not have tumor grade data. In the second half of samples, also 204 had outcome data. Among them, 158 had good outcome and 46 had poor outcome. 13 and 7 patients did not have tumor grade data in good and poor outcome patients respectively.

Two groups of genes (100 Affymetrix® probe-sets each) were identified in 204 training samples which are either correlated or anti-correlated with poor outcome. These two groups of genes are displayed in Tables 35 & 36. Genes in Table 36 are highly enriched for cell cycle and cell proliferation pathways.

A model was built in the training set using a general linear model (from the R package) using the following equation:

Endometrium Cancer Risk Score=Risk Score=0.01786 +0.08208*(prg2−prg1)+(0.14297*Grade) (Formula 15),

where “prg1” is a score calculated from prognosis genes in Table 35 and “prg2” is a score calculated from prognosis genes in Table 36. The scores can be calculated by averaging the log2(intensity) of each probe in the geneset. It's worth pointing out that PGR, ESR1 and AR are all in Table 35, and Table 36 is enriched for proliferation genes. Grade represents tumor grade.

The performance of this model is evaluated in reserved validation set of 184 samples with both gene expression and tumor grade data. FIG. 26 shows the predicted death rate vs. the actual average (running average of 50 samples as ranked by the prediction score) death rate. As shown in the Figure, the model predicts the average death rate very well.

The detailed information about number of samples, number of deaths, and the death rate in each prediction score bin are summarized in Table 37.

TABLE 37
Average death rate versus prediction score.

| Score | Number of samples | Number of death | Death Rate |
|---|---|---|---|
| <0.1 | 67 | 9 | 0.134 |
| 0.1-0.3 | 63 | 11 | 0.175 |
| 0.3-0.5 | 33 | 8 | 0.242 |
| >0.5 | 21 | 11 | 0.524 |

Using a threshold of 0.2, the odds ratio for overall survival is 3.8 (95% CI: 1.8-8.1), Fisher's Exact Test p-value=4.8×10⁻⁴.

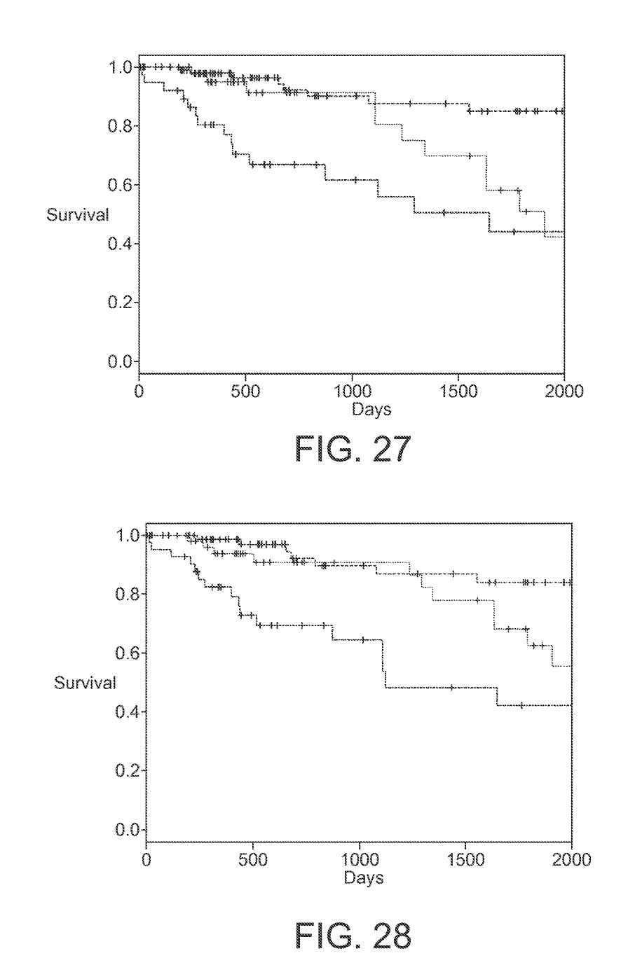

Patients can be further divided into good (risk score<0.2), medium (score 0.2-0.4) and poor (score>0.4) prognosis groups. FIG. 27 shows the Kaplan-Meier curves for these 3 groups. The Chi-square on 2 degrees of freedom is 18.5 (P=9.7×10⁻⁵).

The number of genes in each pathway was reduced to 10 genes.

Prognosis Signature Component 1 (prg1):
Probe IDs: merck-AF016381_a_at, merck-AI918006_at, merck2-NM_001080537_at, merck-NM_145263_at, merck2-NM_173615_at, merck2-XM_371638_at, merck-NM_025145_at, merck2-NM_016930_at, merck-NM_173081_at, merck-AL040975_at
Gene symbols: PGR, UBXN10, SNTN, SPATA18, VWA3A, CDHR4, WDR96, STX18, ARMC3, ESR1

Prognosis Signature Component 2 (prg2):
Probe IDs: merck2-BM904739_at, merck-ENST00000311926_s_at, merck-NM_003875_at, merck-NM_007274_s_at, merck-NM_005225_at, merck-AK027859_s_at, merck-NM_018270_at, merck-NM_198436_s_at, merck2-NM_001168_at, merck2-AF098158_at
Gene symbols: MRGBP, UBE2S, GMPS, ACOT7, E2F1, CENPO, MRGBP, AURKA, BIRC5, TPX2

The scores derived from these 10-genes are correlated to the original scores at the level of 0.96 for prg1, 0.85 for prg2.

Using the reduced gene sets, the updated predictive model is:

Endometrium Cancer Risk Score=Risk Score=−0.13842+0.04180*(prg2−prg1)+(0.18547*Grade) (Formula 16).

Note, the exact coefficients will change depending on the final selection of the technology platform (RNAseq vs. arrays, PCR), and the probe sets or gene lists.

In the validation set, patients are grouped by the prediction score. Table 38 shows the detailed information about number of samples, number of deaths, and the death rate in each prediction score bin.

TABLE 38
Average death rate versus prediction score.

| Score | Number of samples | Number of death | Death Rate |
|---|---|---|---|
| <0.2 | 89 | 10 | 0.112 |
| 0.2-0.4 | 53 | 12 | 0.226 |
| 0.4-0.6 | 36 | 13 | 0.361 |
| >0.6 | 6 | 4 | 0.667 |

Using a threshold of 0.2, the odds ratio for overall survival is 3.5 (95% CI: 1.6-7.6), Fisher's Exact Test p-value=2.1×10⁻³.

Patients can be further divided into good (risk score<0.2), medium (score 0.2-0.4) and poor (score>0.4) prognosis groups. FIG. 28 shows the Kaplan-Meier curves for these 3 groups. The Chi-square on 2 degrees of freedom is 18.4 (P=1.0×10⁻⁴).

TABLE 35
Prognosis signature component 1
(anti-correlated with poor outcome)

| probe | Gene |
|---|---|
| Hmerck-BX106921_at | PGR |
| merck-AL137566_at | PGR |
| merck-AF016381_a_at | PGR |
| merck-AL040975_at | ESR1 |
| merck-ENST00000369936_at | KIAA1324 |
| merck2-AL050116_at | ESR1 |
| merck-BX647987_at | LOC100507053 |
| merck-AL702564_at | PGR |
| merck2-NM_000125_at | ESR1 |
| merck-NM_000125_at | ESR1 |
| merck-AI918006_at | UBXN10 |
| merck2-BX648631_at | UBXN10 |
| merck2-NM_016930_at | STX18 |
| merck-NM_145263_at | SPATA18 |
| merck-NM_001025593_at | ARFIP1 |
| merck-AW970795_at | — |
| merck-NM_152376_s_at | UBXN10 |
| merck2-AI288607_at | — |
| merck2-M69297_at | — |
| merck-NM_020775_s_at | KIAA1324 |
| merck2-BM695584_at | ARHGAP26 |
| merck2-NM_006961_at | ZNF19 |
| merck-NM_013367_s_at | ANAPC4 |
| merck-NM_000266_at | NDP |
| merck-NM_025059_at | CCDC170 |
| merck-CR609491_a_at | STX18 |
| merck2-NM_005327_at | HADH |
| merck-ENST00000324607_s_at | MBOAT1 |
| merck2-CA309763_at | NDP |
| merck-ENST00000369949_s_at | C1orf194 |
| merck-NM_014668_s_at | GREB1 |
| merck-NM_025145_at | WDR96 |
| merck-NM_001002912_s_at | C1orf173 |
| merck2-ENST00000342217_at | C1orf173 |
| merck2-AK025905_at | SOX17 |
| merck-BC094795_a_at | PIK3R1 |
| merck2-BG619802_at | EYA2 |
| merck-NM_015071_at | ARHGAP26 |
| merck-BX648957_at | LOC100505776 |
| merck-BC028018_at | LOC100129098 |
| merck-NM_178456_at | C20orf85 |
| merck-NM_022454_at | SOX17 |
| merck-ENST00000347491_s_at | ESR1 |
| merck-NM_214462_at | DACT2 |
| merck-NM_003551_at | NME5 |
| merck-ENST00000319471_a_at | SORBS2 |
| merck2-AM392558_at | SORBS2 |
| merck2-CB999963_at | RNF180 |
| merck-NM_181523_at | PIK3R1 |
| merck-NM_018242_at | SLC47A1 |
| merck-AK057330_a_at | ZNF19 |
| merck-NM_022123_a_at | NPAS3 |
| merck2-BQ894504_at | PIK3R1 |
| merck-BC063677_at | TMEM231 CHST5 |
| merck-NM_145170_at | TTC18 |
| merck-BC063866_at | COL28A1 |
| merck-NM_003774_at | POC1B-GALNT4 GALNT4 |
| merck-NM_018043_at | ANO1 |
| merck2-AY358612_at | TMEM231 CHST5 |
| merck-AF085947_at | NPAS3 |
| merck-NM_015460_at | MYRIP |
| merck2-DT217746_at | ASRGL1 |
| merck2-AK225360_at | SLC47A1 |
| merck2-NM_001080537_at | SNTN |
| merck-CF453637_s_at | NPAS3 |
| merck2-BX093691_at | TTC18 |
| merck-NM_004816_s_at | FAM189A2 |
| merck-ENST00000299840_s_at | VWA3A |
| merck-BC037328_at | MAP2K6 |
| merck-AL832580_at | RNF180 |
| merck2-NM_144722_at | SPEF2 |
| merck-NM_005244_at | EYA2 |
| merck-NM_025080_s_at | ASRGL1 |
| merck-AI624058_at | FAM216B |
| merck2-ENST00000374690_at | AR |
| merck-NM_018091_s_at | ELP3 |
| merck-XM_942673_at | SNTN |
| merck2-BX648791_at | — |
| merck-CD687039_a_at | DNAH12 |
| merck2-BQ684833_at | ACSL5 |
| merck2-BX096668_at | — |
| merck-AY312852_s_at | GTF2IRD2 GTF2IRD2B GTF2I |
| merck-NM_145058_at | RILPL2 |
| merck-NM_201520_s_at | SLC25A35 RANGRF |
| merck-BC047078_at | SLC25A15 |
| merck2-NM_173615_at | VWA3A |
| merck-NM_015058_at | VWA8 |
| merck2-NM_173537_s_at | — |
| merck2-NM_001003795_s_at | — |
| merck-T68445_a_at | AR |
| merck2-XM_371638_at | CDHR4 |
| merck2-BC026182_at | NME5 |
| merck-NM_005397_at | PODXL MKLN1 |
| merck-NM_001029875_at | RGS7BP |
| merck-NM_015271_at | TRIM2 |
| merck2-BC047091_a_at | ZNF19 |
| merck2-AA148029_at | PODXL MKLN1 |
| merck2-NM_145283_at | NXNL2 |
| merck-AL050026_at | PALLD |
| merck-NM_020879_s_at | CCDC146 |

TABLE 36
Prognosis signature component 2 (correlated with poor outcome)

| probe | Gene |
|---|---|
| merck2-BM904739_at | MRGBP |
| merck-NM_018270_at | MRGBP |
| merck-NM_007274_s_at | ACOT7 |
| merck-NM_004358_at | CDC25B |
| merck2-BQ437524_at | CDC25B |
| merck-AF533230_x_at | USP32 |
| merck2-BX647988_a_at | CDC25B |
| merck2-BC007074_a_at | TNNT1 |
| merck2-BC001395_at | CIAO1 |
| merck2-ENST00000356433_at | DLL3 |
| merck-BX442394_a_at | SOX11 |
| merck2-BQ644821_at | — |
| merck2-AK026140_at | — |
| merck-XM_926989_s_at | ACAA2 |
| merck-CR609746_a_at | C17orf96 |
| merck-NM_138570_s_at | SLC38A10 |
| merck-NM_001010911_at | CASC10 |
| merck2-AY762903_at | TNNT1 |
| merck-NM_003283_s_at | TNNT1 |
| merck2-DQ893376_s_at | ACAA2 |
| merck2-BC002615_at | CSNK2A1 CSNK2A3 |
| merck-NM_001031713_s_at | MCUR1 |
| merck-BC003580_s_at | CIAO1 |
| merck-NM_003108_at | SOX11 |
| merck-NM_021972_at | SPHK1 |
| merck2-DQ893376_at | ACAA2 |
| merck-NM_004181_at | UCHL1 |
| merck-BC037270_a_at | AKAP8 |
| merck-NM_001039467_s_at | RGS19 |
| merck-NM_203486_s_at | DLL3 |
| merck-NM_153485_at | NUP155 |
| merck-ENST00000311926_s_at | UBE2S |
| merck-NM_006111_at | ACAA2 |
| merck-NM_004708_s_at | PDCD5 |
| merck-NM_021158_at | TRIB3 |
| merck-ENST00000381973_s_at | CSNK2A1 CSNK2A3 |
| merck-NM_000071_s_at | CBS U2AF1 |
| merck-NM_004209_at | SYNGR3 |
| merck-NM_152310_at | ELOVL3 PITX3 |
| merck-NM_004112_at | FGF11 CHRNB1 |
| merck2-BI602361_s_at | — |
| merck2-BC068553_at | DR1 |
| merck-DW451489_s_at | MED8 |
| merck-NM_002808_at | PSMD2 |
| merck-CR610223_a_at | SCARB2 |
| merck-NM_003875_at | GMPS |
| merck-BC028386_a_at | RRP1B |
| merck-CR619305_a_at | GNB1 |
| merck-NM_000022_at | ADA |
| merck-CR592459_a_at | MAPRE1 |
| merck2-BC030582_at | TCP11L1 |
| merck2-BC002615_s_at | CSNK2A1 CSNK2A3 |
| merck-NM_001089_at | ABCA3 |
| merck-NM_015122_at | FCHO1 |
| merck-NM_001281_at | TBCB |
| merck-NM_001489_a_at | NR6A1 |
| merck-AK023842_a_at | BAZ2A |
| merck-NM_002792_s_at | PSMA7 |
| merck-BC025264_a_at | YTHDF1 |
| merck-NM_001426_at | EN1 |
| merck-NM_003198_at | TCEB3 |
| merck2-ENST00000305989_at | FTL GYS1 |
| merck-AK027859_s_at | CENPO |
| merck-ENST00000264607_a_at | ASB1 |
| merck-NM_013409_at | FST |
| merck-NM_080618_at | CTCFL |
| merck2-BQ227259_at | SCARB2 |
| merck-BX649059_at | GAS2L3 |
| merck-NM_152699_s_at | SENP5 |
| merck-NM_014109_a_at | ATAD2 |
| merck-AK126101_a_at | PLXNA1 |
| merck-NM_004341_at | CAD |
| merck2-NM_001079862_at | DBI |
| merck-NM_013321_at | SNX8 |
| merck2-EF560732_a_at | CKAP2 |
| merck-CR617826_a_at | TIMM50 |
| merck2-BC007338_at | CDV3 |
| merck-NM_206831_a_at | DPH3 OXNAD1 RFTN1 |
| merck2-ENST00000374536_at | TCEB3 |
| merck-NM_007224_at | NXPH4 SHMT2 |
| merck-ENST00000373683_s_at | SKA2 |
| merck2-AA169659_s_at | — |
| merck2-BC121146_at | TIMM50 |
| merck2-ENST00000305989_x_at | FTL GYS1 |
| merck-BM722157_a_at | SOX11 |
| merck-BM909568_s_at | PRMT2 S100B |
| merck2-BC025843_at | L1CAM |
| merck-NM_024871_at | MAP6D1 |
| merck2-BE264170_at | PLCXD1 |
| merck-NM_003088_at | FSCN1 |
| merck2-AK025810_at | WDR5 |
| merck2-BM674474_at | — |
| merck-BU145850_at | — |
| merck2-AK222554_at | SF3A3 |
| merck2-AF225416_at | SPC25 |
| merck-NM_198207_at | CERS1 |
| merck2-AI149996_at | ADRM1 |
| merck-NM_000175_s_at | GPI |
| merck-AK074937_a_at | NETO2 |
| merck-ENST00000330234_a_at | DGCR5 |

### Example 9

### Prognostic Model for Melanoma

This example describes a melanoma prognosis model based on gene expression profiling data. The model contains two gene expression signatures as components. In the second part of the example, the number of genes in each signature is reduced to 10 genes to simplify the implementation of this prognosis model.

A total of 711 samples were profiled by Affymetrix® expression arrays, of which 559 were malignant melanoma. A composite model was built using the first half of samples and the model validated using the second half of samples. In the first half of samples, 292 samples had outcome data (alive or dead). Among them, 123 had good outcome and 169 had poor outcome. In the second half of samples, all 267 had outcome data. Among them, 105 had good outcome and 162 had poor outcome. Besides malignant melanoma, there are also 152 other skin cancer samples including squamous cell carcinoma, Merkel cell carcinoma, Basal cell carcinoma, etc. The model developed by malignant melanoma was also evaluated in these 152 samples.

Two groups of genes (100 Affymetrix® probe-sets each) were identified in 267 training samples which are either correlated or anti-correlated with poor outcome. These two groups of genes are displayed in Tables 37 & 38. Genes in Table 38 are highly enriched for cell cycle and cell proliferation pathways.

A model was built in the training set using a general linear model (from the R package) using the following equation:

Melanoma Cancer Risk Score=Risk Score=0.16708+0.10739*(prg2−prg1) (Formula 17),

where “prg1” is a score calculated from prognosis genes in Table 37 and “prg2” is a score calculated from prognosis genes in Table 38. The scores can be calculated by averaging the log2(intensity) of each probe in the geneset.

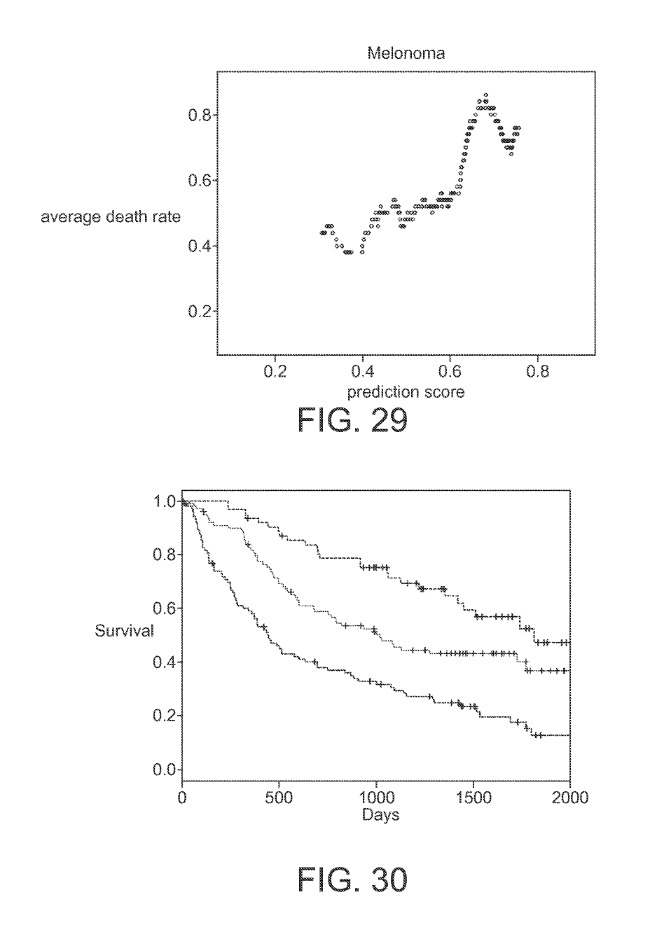

The performance of this model is evaluated in reserved validation set of 267 samples with also the stage data. FIG. 29 shows the predicted death rate vs. the actual average (running average of 50 samples as ranked by the prediction score) death rate. As shown in the Figure, the model predicts the average death rate very well.

The detailed information about number of samples, number of deaths, and the death rate in each prediction score bin are summarized in Table 38.

TABLE 38
Average death rate versus prediction score.

| Score | Number of samples | Number of death | Death Rate |
|---|---|---|---|
| <0.4 | 45 | 18 | 0.400 |
| 0.4-0.5 | 32 | 15 | 0.469 |
| 0.5-0.6 | 47 | 24 | 0.511 |
| 0.6-0.7 | 66 | 49 | 0.742 |
| >0.7 | 77 | 56 | 0.727 |

Using a threshold of 0.58, the odds ratio for overall survival is 3.0, 95% CI: 1.8-5.0, Fisher's Exact Test p-value=2.5×10⁻⁵.

Patients can be further divided into good (risk score<0.45), medium (score 0.45-0.65) and poor (score>0.65) prognosis groups. FIG. 30 shows the Kaplan-Meier curves for these 3 groups. The Chi-square on 2 degrees of freedom is 37.0 (P=9.3×10⁻⁹).

The number of genes in each pathway was reduced to 10 genes.

Prognosis Signature Component 1 (prg1):
Probe IDs: merck-AK128436_at, merck-NM_000073_at, merck-NM_002351_s_at, merck2-NM_052931_at, merck-NM_000734_at, merck-NM_052931_at, merck-NM_018556_s_at, merck2-NM_025228_at, merck2-NM_001010923_at, merck-NM_198517_at
Gene symbols: IKZF3, CD3G, SH2D1A, SLAMF6, CD247, SLAMF6, SIRPG, TRAF3IP3, THEMIS, TBC1D10C

Prognosis Signature Component 2 (prg2):
Probe IDs: merck-NM_032039_at, merck-NM_001010866_at, merck2-AL157485_at, merck-ENST00000336690_s_at, merck-NM_014291_at, merck-NM_001014832_s_at, merck-BM981759_a_at, merck-ENST00000372943_at, merck-ENST00000360797_s_at, merck2-CA311625_at
Gene symbols: ITFG3, TMEM201, TBC1D16, PPT2, GCAT, PAK4, OTUD7B, FITM2, PCGF2, GCAT

The scores derived from these 10-genes are correlated to the original scores at the level of 0.98 for prg1, 0.87 for prg2.

Using the reduced gene sets, the updated predictive model is:

Melanoma Cancer Risk Score=Risk Score=0.43492+0.06120*(prg2−prg1) (Formula 18).

Note, the exact coefficients will change depending on the final selection of the technology platform (RNAseq vs. arrays, PCR), and the probe sets or gene lists.

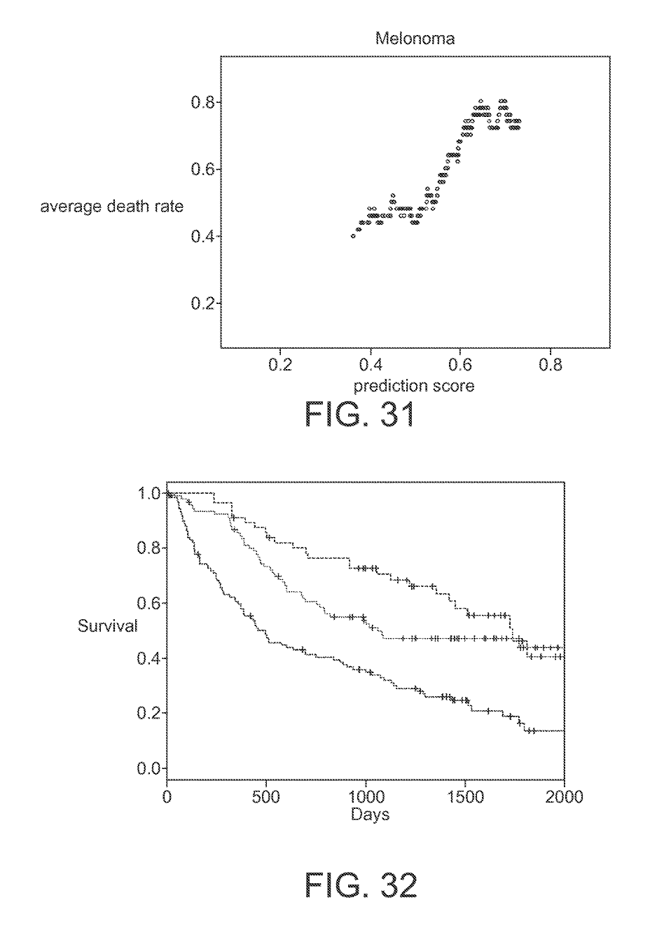

FIG. 31 shows the predicted death rate vs. the actual average (running average of 50 samples as ranked by the prediction score) death rate for this updated model. As shown in the Figure, the model predicts the average death rate very well.

The detailed information about number of samples, number of deaths, and the death rate in each prediction score bin are summarized in Table 39.

TABLE 39
Average death rate versus prediction score.

| Score | Number of samples | Number of death | Death Rate |
|---|---|---|---|
| <0.4 | 36 | 14 | 0.389 |
| 0.4-0.5 | 46 | 24 | 0.522 |
| 0.5-0.6 | 66 | 34 | 0.515 |
| 0.6-0.7 | 69 | 53 | 0.768 |
| >0.7 | 50 | 37 | 0.740 |

Using a threshold of 0.6, the odds ratio for overall survival is 3.3 (95% CI: 1.9-5.6), Fisher's Exact Test p-value=8.9×10⁻⁶.

Patients can be further divided into good (risk score<0.45), medium (score 0.45-0.6) and poor (score>0.6) prognosis groups. FIG. 32 shows the Kaplan-Meier curves for these 3 groups. The Chi-square on 2 degrees of freedom is 32.2 (P=1.0×10⁻⁷).

The Model is predictive in other skin cancers: Besides malignant melanoma, there are also 152 other skin cancer samples including squamous cell carcinoma, Merkel cell carcinoma, Basal cell carcinoma, etc. The same model was applied to these 152 samples to evaluate its predictive power.

At a threshold of 0.45, the odds ratio is 5.4, 95% CI: 1.9-15.1, Fisher's exact P-value is 6.3×10⁻⁴.

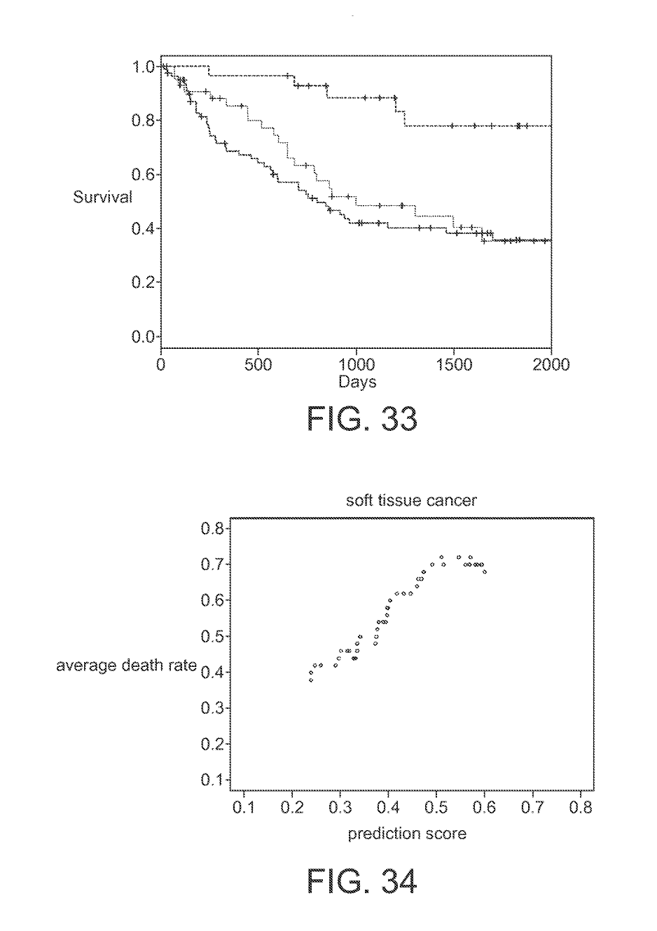

FIG. 33 shows the Kaplan-Meier curves when patients are divided into 3 groups (<0.45, 0.45-0.6 and >0.6). The Chi-square for 2 degrees of freedom is 14 (P=9.2×10⁻⁴).

TABLE 37
Prognosis signature component 1 (anti-correlated with poor outcome)

| probe | Gene |
|---|---|
| merck-AI912585_at | — |
| merck-AK124031_a_at | THEMIS |
| merck-NM_016388_at | TRAT1 |
| merck2-AY292266_at | — |
| merck-NM_173799_at | TIGIT |
| merck-NM_000619_at | IFNG |
| merck-NM_002351_s_at | SH2D1A |
| merck-NM_001001895_at | UBASH3A |
| merck-NM_012092_at | ICOS |
| merck-ENST00000383671_a_at | TIGIT |
| merck2-ENST00000390352_x_at | — |
| merck-Z22965_s_at | — |
| merck2-NM_004931_a_at | CD8B |
| merck-BC036924_at | PATL2 SPG11 |
| merck-NM_000073_at | CD3G |
| merck2-U39114_s_at | — |
| merck-NM_198333_s_at | P2RY10 |
| merck-DT807100_at | CD3D CD3G |
| merck2-AY292266_x_at | — |
| merck2-BX108263_at | LOC101929510 LOC101929531 |
| merck2-ENST00000390435_x_at | TRAV8-3 MGC40069 |
| merck-NM_013308_at | GPR171 |
| merck-BX648371_at | LINC00861 |
| merck2-NM_001010923_at | THEMIS |
| merck-ENST00000206681_at | — |
| merck2-NM_152615_at | PARP15 |
| merck-Z75948_s_at | TRAV14DV4 |
| merck-CD700761_s_at | PPP1R16B |
| merck2-ENST00000390353_at | IFI6 TRBV6-1 |
| merck2-ENST00000390352_at | — |
| merck2-ENST00000390400_at | TRBV28 |
| merck2-BM677447_at | MIAT |
| merck-NM_172101_at | CD8B |
| merck-NM_152693_a_at | FAM226A FAM226B |
| merck-AK124004_at | AKAP5 |
| merck2-AF459027_at | FCRL3 |
| merck-NM_003151_a_at | STAT4 |
| merck2-AY006176_x_at | — |
| merck2-AW170566_at | — |
| merck2-ENST00000390386_a_at | TRBV12-3 TRBV12-4 |
| merck2-ENST00000390363_at | — |
| merck-CR597260_at | LOC101059954 |
| merck-AK097158_at | LINC00996 |
| merck2-ENST00000390454_at | — |
| merck-ENST00000341173_s_at | TRAF3IP3 |
| merck2-NM_025228_at | TRAF3IP3 |
| merck-NM_032553_at | GPR174 |
| merck2-X92770_x_at | — |
| merck-BC040064_at | ITGB2-AS1 ITGB2 |
| merck-ENST00000316577_s_at | TESPA1 |
| merck2-ENST00000390439_at | — |
| merck2-AJ007770_at | — |
| merck-NM_014450_at | SIT1 RMRP |
| merck-AK127925_at | CD2 |
| merck-ENST00000303432_a_at | CD8B |
| merck2-ENST00000390387_a_at | TRBV12-3 TRBV12-4 |
| merck2-AF532855_x_at | — |
| merck2-ENST00000390435_at | TRAV8-3 MGC40069 |
| merck2-ENST00000390449_at | — |
| merck2-ENST00000390350_at | — |
| merck2-ENST00000390433_at | — |
| merck2-ENST00000390393_at | TRBV19 |
| merck-Y15200_s_at | — |
| merck-AK098833_s_at | MIAT |
| merck-AY190088_s_at | — |
| merck-AI281804_at | GPR174 |
| merck2-M27337_x_at | TRGV2 TRGV4 |
| merck2-L01087_at | PRKCQ |
| merck-AF327297_s_at | TRAJ17 |
| merck-AK128436_at | IKZF3 |
| merck2-ENST00000390394_s_at | — |
| merck2-ENST00000390359_x_at | TRBV4-2 TRBV7-2 |
| merck2-Z22966_a_at | — |
| merck-NM_005292_at | GPR18 |
| merck2-NM_001006638_at | RAB37 SLC9A3R1 |
| merck-NM_002262_at | KLRD1 |
| merck-NM_152781_at | C17orf66 |
| merck-NM_000732_at | CD3D |
| merck-NM_000639_at | FASLG |
| merck-NM_153615_s_at | RGL4 |
| merck2-ENST00000390359_at | TRBV4-2 TRBV7-2 |
| merck2-AJ007771_at | TRAV8-6 |
| merck-NM_014716_at | ACAP1 |
| merck-NM_032206_a_at | NLRC5 |
| merck-NM_001024667_s_at | FCRL3 |
| merck-NM_198517_at | TBC1D10C |
| merck2-ENST00000390353_x_at | IFI6 TRBV6-1 |
| merck-NM_000595_a_at | LTA |
| merck-BF870822_at | — |
| merck-ENST00000379833_at | GVINP1 |
| merck2-ENST00000390442_at | TRAV12-3 |
| merck2-AF129512_at | IKZF3 |
| merck-NM_006566_at | CD226 |
| merck-AK095686_s_at | MIAT |
| merck-BC028218_a_at | ZBP1 |
| merck-NM_006257_at | PRKCQ |
| merck-NM_018556_s_at | SIRPG |
| merck-AI203370_at | GBP5 |
| merck2-NM_001005176_a_at | SP140 |
| merck-BM700951_at | KLRK1 KLRC4-KLRK1 |

TABLE 38
Prognosis signature component 2 (correlated with poor outcome)

| probe | Gene |
|---|---|
| merck-NM_005027_s_at | PIK3R2 |
| merck-NM_001015055_s_at | RTKN |
| merck2-BT019930_a_at | — |
| merck2-BC001528_at | — |
| merck2-NM_178121_at | MEGF8 |
| merck2-NM_003250_a_at | THRA NR1D1 |
| merck-NM_178148_at | SLC35B2 HSP90AB1 |
| merck-NM_178121_at | MEGF8 |
| merck-NM_181521_at | CMTM4 |
| merck-CR619245_a_at | BSG |
| merck2-AB018267_at | IPO13 |
| merck-AK222827_a_at | GGCX |
| merck2-BM464059_at | — |
| merck2-NM_198591_at | BSG |
| merck-H05603_a_at | THRA NR1D1 |
| merck2-NM_001078172_at | FAM127B |
| merck-AF086201_at | TMEM63B |
| merck-NM_032039_at | ITFG3 |
| merck-NM_003872_s_at | NRP2 |
| merck-NM_004793_s_at | LONP1 RPL36 |
| merck-ENST00000375101_a_at | AGPAT1 |
| merck-NM_018426_at | TMEM63B |
| merck-NM_001069_at | TUBB2A |
| merck-NM_032806_at | POMGNT2 |
| merck-NM_003051_at | SLC16A1 |
| merck-AK128554_at | IRGQ |
| merck2-CX758384_at | DDR1 |
| merck-NM_024085_at | ATG9A ABCB6 |
| merck-NM_032088_s_at | PCDHGA1 PCDHGA10 |
| | PCDHGA11 PCDHGA12 |
| | PCDHGA2 PCDHGA3 |
| | PCDHGA4 PCDHGA5 |
| | PCDHGA6 PCDHGA7 |
| | PCDHGA8 PCDHGA9 |
| | PCDHGB1 PCDHGB2 |
| | PCDHGB3 PCDHGB4 |
| | PCDHGB5 PCDHGB6 |
| | PCDHGB7 PCDHGC3 |
| | PCDHGC4 PCDHGC5 |
| merck-NM_001954_a_at | DDR1 |
| merck-NM_015388_s_at | YIPF3 |
| merck-NM_014623_at | MEA1 |
| merck-ENST00000372943_at | FITM2 |
| merck-NM_004053_at | BYSL |
| merck-NM_018028_at | SAMD4B |
| merck-NM_001012981_at | ZKSCAN2 |
| merck-ENST00000321333_x_at | FAM127B |
| merck2-BU553968_x_at | — |
| merck2-NM_000821_at | GGCX |
| merck-NM_006876_at | B3GNT1 |
| merck-ENST00000261497_at | USP22 |
| merck-ENST00000372235_a_at | TMEM53 |
| merck2-BC016713_a_at | PARVA |
| merck-BC001048_s_at | CDK16 |
| merck2-NM_003250_at | — |
| merck-ENST00000263381_a_at | WIZ |
| merck-ENST00000336690_s_at | PPT2 |
| merck-NM_001410_at | MEGF8 |
| merck-NM_004854_at | CHST10 |
| merck-ENST00000360797_s_at | PCGF2 |
| merck-AI263624_a_at | POFUT1 |
| merck-NM_001035507_a_at | AGBL5 |
| merck-NM_001024736_s_at | CD276 |
| merck-CR624090_a_at | PARVA |
| merck-NM_004860_at | FXR2 |
| merck2-AK055481_at | SAE1 |
| merck2-BI093105_at | NR1I2 |
| merck-NM_016223_at | PACSIN3 |
| merck2-NM_024103_x_at | SLC25A23 |
| merck-NM_005689_at | ABCB6 |
| merck-NM_182980_at | OSGIN1 |
| merck-ENST00000313594_x_at | GCSH LOC101060817 |
| merck-NM_006062_at | SMYD5 |
| merck2-NM_005035_at | POLRMT |
| merck-NM_001014832_s_at | PAK4 |
| merck2-BM970572_at | OTUD7B |
| merck-NM_001492_s_at | CERS1 |
| merck2-ENST00000358681_at | EXT2 |
| merck-NM_012476_at | VAX2 ATP6V1B1 |
| merck-NM_020378_at | NAT14 |
| merck2-AK026006_a_at | TMEM53 |
| merck-NM_004082_at | DCTN1 |
| merck2-NM_005789_at | PSME3 AOC2 |
| merck2-NM_014015_at | — |
| merck2-AL832023_at | POFUT1 |
| merck-NM_017802_s_at | HEATR2 |
| merck-BC072383_s_at | NPAS2 |
| merck2-BC002515_s_at | — |
| merck-CD014070_s_at | TUBG2 |
| merck-NM_001040716_at | PC |
| merck-NM_006690_s_at | MMP24 |
| merck2-CR600560_at | EMC8 |
| merck-NM_180976_at | PPP2R5D |
| merck-NM_015277_s_at | NEDD4L |
| merck-NM_178012_at | TUBB2B |
| merck2-AF059195_at | MAFG |
| merck-NM_001182_at | ALDH7A1 PDE8B |
| merck-NM_004422_at | DVL2 ACADVL |
| merck2-CK821133_a_at | — |
| merck-NM_003780_at | B4GALT2 |
| merck-ENST00000334310_a_at | TEAD1 |
| merck-NM_005234_at | NR2F6 |
| merck2-AF147421_at | ARHGAP5-AS1 |
| merck-AY672105_a_at | POLRMT CYP4F11 CYP4F2 |
| merck-NM_016147_s_at | PPME1 |
| merck-NM_032829_at | FAM222A |
| merck-NM_152600_at | ZNF579 |
| merck-NM_001037131_at | AGAP1 |
| merck-NM_017797_s_at | BTBD2 |
| merck-BC005142_a_at | AP3D1 |

### Example 10

### Prognostic Model for Soft Tissue Cancer

This example describes a soft tissue cancer prognosis model based on gene expression profiling data. The model contains two gene expression signatures as components. In the second part of the example, the number of genes in each signature is reduced to 10 genes to simplify the implementation of this prognosis model. Since both the prognosis signatures derived from the current dataset and the pre-defined proliferation signature predict patient outcome, both predictors were combined.

A total of 190 samples were profiled by Affymetrix® expression arrays. A composite model was built using the first half of samples and the model validated using the second half of samples. In the first half of samples, 261 samples had outcome data (live or death). In the first half of samples, 95 samples had outcome data (alive or dead). Among them, 49 had good outcome and 46 had poor outcome. 11 of the 49 good outcome patients did not have detailed last follow-up dates. In the second half of samples, all 95 had outcome data. Among them, 46 had good outcome and 49 had poor outcome. 5 out of the 46 good outcome patients did not have detailed follow-up dates.

Two groups of genes (100 Affymetrix® probe-sets each) were identified in 95 training samples which are either correlated or anti-correlated with poor outcome. These two groups of genes are displayed in Tables 40 & 41.

A model was built in the training set using a general linear model (from the R package) using the following equation:

Soft Tissue Cancer Risk Score=Risk Score=0.39820+0.30357*(prg2−prg1) (Formula 19),

where “prg1” is a score calculated from prognosis genes in Table 40 and “prg2” is a score calculated from prognosis genes in Table 41. The scores can be calculated by averaging the log2(intensity) of each probe in the geneset.

The performance of this model is evaluated in reserved validation set of 95 samples. FIG. 34 shows the predicted death rate vs. the actual average (running average of 50 samples as ranked by the prediction score) death rate. As shown in the Figure, the model predicts the average death rate very well.

The detailed information about number of samples, number of deaths, and the death rate in each prediction score bin are summarized in Table 42.

TABLE 42
Average death rate versus prediction score.

| Score | Number of samples | Number of death | Death Rate |
|---|---|---|---|
| <0.2 | 20 | 0 | 0.000 |
| 02.-0.4 | 29 | 14 | 0.483 |
| 0.4-0.6 | 20 | 13 | 0.650 |
| >0.6 | 26 | 18 | 0.692 |

Using a threshold of 0.34, the odds ratio for overall survival is 6.9, 95% CI: 2.7-17.6, Fisher's Exact Test p-value=2.4×10⁻⁵.

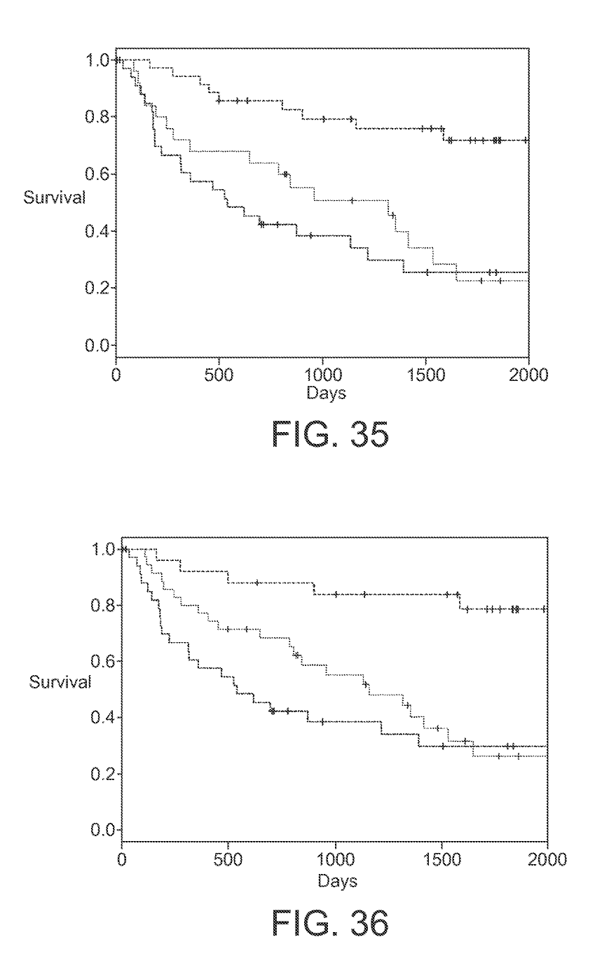

Patients can be further divided into good (risk score<0.34), medium (score 0.34-0.55) and poor (score>0.55) prognosis groups. FIG. 35 shows the Kaplan-Meier curves for these 3 groups. The Chi-square on 2 degrees of freedom is 18.3 (P=1.1×10′).

The number of genes in each pathway was reduced to 10 genes.

Prognosis Signature Component 1 (prg1):
Probe IDs: merck2-CN308012_at, merck-NM_003617_at, merck-NM_001981_at, merck-NM_014774_at, merck-NM_033439_at, merck-NM_017719_at, merck-NM_012158_at, merck2-AA551214_a_at, merck-BC030112_at, merck2-ENST00000377993_at

Gene symbols: EFCAB14, RGS5, EPS15, EFCAB14, IL33, SNRK, FBXL3, MBNL1, HIPK3, CMAHP

Prognosis Signature Component 2 (prg2):
Probe IDs: merck-CR407609_a_at, merck2-NM_005782_at, merck-BI084560_s_at, merck-BC066298_a_at, merck-ENST00000311926_s_at, merck-NM_003860_s_at, merck2-BM504304_a_at, merck2-XM_001134348_at, merck2-DC428989_at, merck-BG504479_s_at
Gene symbols: MRPS12, ALYREF, SNRPB, LSM12, UBE2S, BANF1, LSM4, ANAPC11, HNRNPK, RANBP1

The scores derived from these 10-genes are correlated to the original scores at the level of 0.92 for prg1, 0.94 for prg2.

Using the reduced gene sets, the updated predictive model is:

Soft Tissue Cancer Risk Score=0.74291+0.16726*(prg2−prg1) (Formula 20).

Note, the exact coefficients will change depending on the final selection of the technology platform (RNAseq vs. arrays, PCR), and the probe sets or gene lists.

Patients in the validation set are grouped by the prediction score. Table 43 shows the detailed information about number of samples, number of deaths, and the death rate in each prediction score bin.

TABLE 43
Average death rate versus prediction score.

| Score | Number of samples | Number of death | Death Rate |
|---|---|---|---|
| <0.2 | 12 | 2 | 0.167 |
| 0.2-0.4 | 26 | 9 | 0.346 |
| 0.4-0.6 | 32 | 22 | 0.688 |
| >0.6 | 25 | 16 | 0.640 |

Using a threshold of 0.34, the odds ratio for overall survival is 7.4 (95% CI: 2.5-22.0), Fisher's Exact Test p-value=1.6×10⁻⁴.

Patients can be further divided into good (risk score<0.34), medium (score 0.34-0.55) and poor (score>0.55) prognosis groups. FIG. 36 shows the Kaplan-Meier curves for these 3 groups. The Chi-square on 2 degrees of freedom is 16.1 (P=3.2×10-4).

A predefined proliferation signature (Table 44) is also prognostic in soft tissue cancer patients. The correlation of the proliferation score and the Risk Score of Formula 20 in soft tissue patients is 0.51.

The model was built in the training set using a general linear model (from the R package) with the following components:

Soft Tissue Cancer Risk Score=−0.32072+0.10405*pscore (Formula 21).

Where pscore is the score calculated from prognosis genes in Table 44 by averaging the log2(intensity) of each probe in the geneset.

The performance of this model is evaluated in reserved validation set of 95 samples. FIG. 37 shows the predicted death rate vs. the actual average (running average of 50 samples as ranked by the prediction score) death rate. As shown in the Figure, the model predicts the average death rate very well.

The detailed information about number of samples, number of deaths, and the death rate in each prediction score bin are summarized in Table 45.

TABLE 45
Average death rate versus prediction score.

| Score | Number of samples | Number of death | Death Rate |
|---|---|---|---|
| <0.4 | 23 | 3 | 0.130 |
| 0.4-0.5 | 20 | 10 | 0.500 |
| 0.5-0.6 | 24 | 16 | 0.667 |
| >0.6 | 28 | 20 | 0.714 |

Using a threshold of 0.42, the odds ratio for overall survival is 7.4, 95% CI: 2.5 -22.0, Fisher's Exact Test p-value=1.6×10⁻⁴.

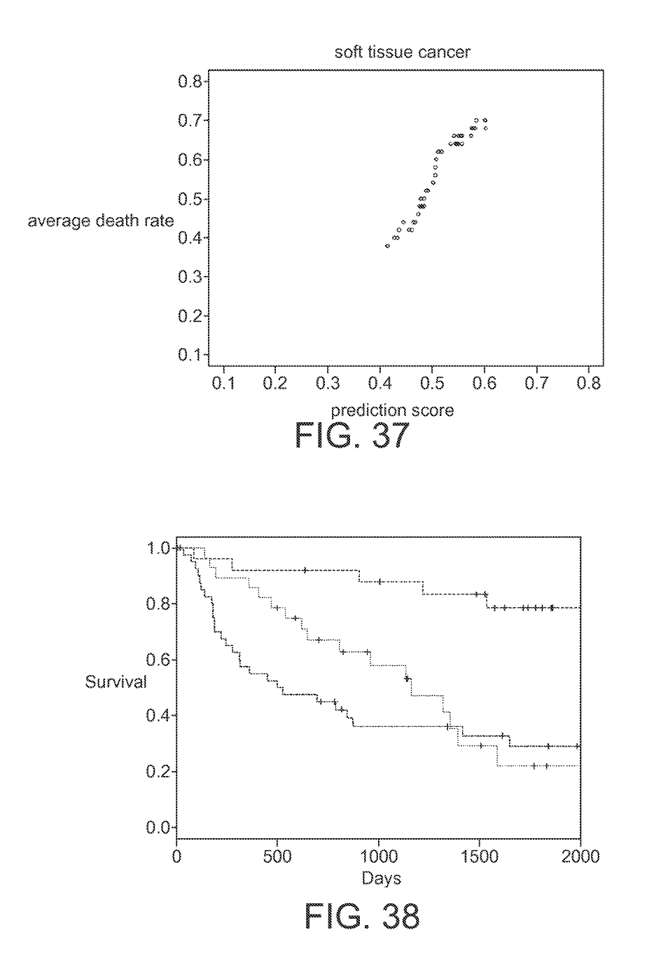

Patients can be further divided into good (risk score<0.42), medium (score 0.42-0.55) and poor (score>0.55) prognosis groups. FIG. 38 shows the Kaplan-Meier curves for these 3 groups. The Chi-square on 2 degrees of freedom is 16.8 (P=2.3×10⁻⁴).

The number of genes in proliferation signature can be reduced to 10 genes.
Probe IDs: merck-NM_012112_at, merck-NM_004701_at, merck-NM_001809_at, merck-NM_145060_at, merck-CR602926_s_at, merck-U63743_a_at, merck-NM_018101_at, merck2-AK000490_a_at, merck-NM_080668_at, merck-ENST00000333706_x_at
Gene symbols: TPX2, CCNB2, CENPA, SKA1, CCNB1, KIF2C, CDCA8, DEPDC1, CDCA5, BIRC5

The scores derived from these 10-genes are correlated to the original scores at the level of 0.99.

Using the reduced gene sets, the updated predictive model is:

Soft Tissue Cancer Risk Score=−0.24302+0.08483*pscore (Formula 22).

Note, the exact coefficients will change depending on the final selection of the technology platform (RNAseq vs. arrays, PCR), and the probe sets or gene lists.

In the validation set, the detailed information about number of samples, number of deaths, and the death rate in each prediction score bin are summarized in Table 46.

TABLE 46
Average death rate versus prediction score.

| Score | Number of samples | Number of death | Death Rate |
|---|---|---|---|
| <0.4 | 21 | 3 | 0.143 |
| 0.4-0.5 | 20 | 11 | 0.550 |
| 0.5-0.6 | 29 | 19 | 0.655 |
| >0.6 | 25 | 16 | 0.640 |

Using a threshold of 0.40, the odds ratio for overall survival is 9.9 (95% CI: 2.7-36.5), Fisher's Exact Test p-value=1.3×10⁻⁴.

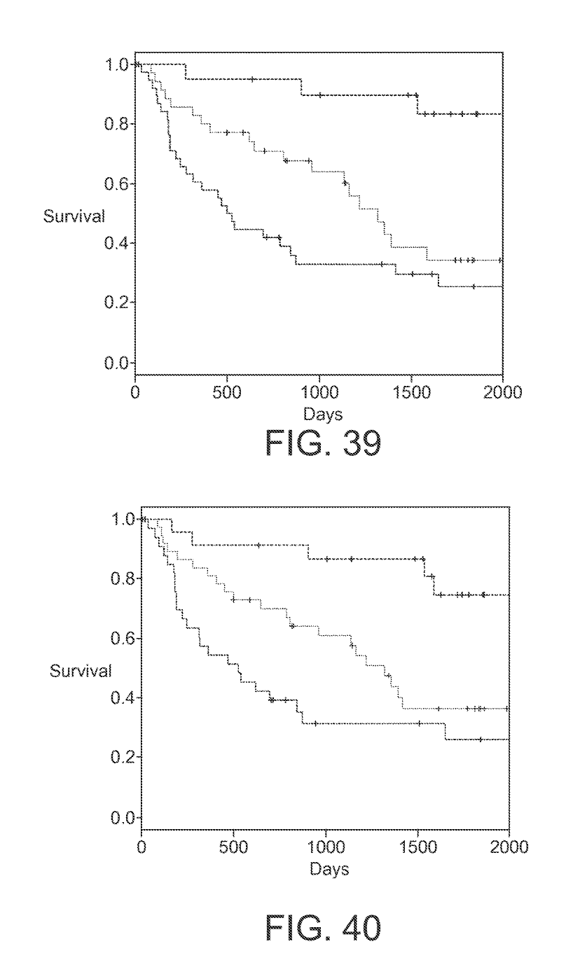

Patients can be further divided into good (risk score<0.4), medium (score 0.4-0.55) and poor (score>0.55) prognosis groups. FIG. 39 shows the Kaplan-Meier curves for these 3 groups. The Chi-square on 2 degrees of freedom is 18.0 (P=1.2×10⁻⁴).

The two models (Formula 20 and Formula 22) can be combined to a single model to predict patient outcome. The combination can be done either by averaging the prediction scores, or by counting the risk factors.

FIG. 40 shows the Kaplan-Meier plot using the average risk score RS:

Soft Tissue Cancer Risk Score=(RS1+RS2)/2 (Formula 23).

Where RS1 is the risk score from Formula 20 and RS2 the risk score from Formula 22. When patients in the validation set were binned into three groups (<0.4, 0.4-0.55, and >0.55), the Chi-square on 2 degrees of freedom is 16.4 (P=2.7×10⁻⁴).

Alternatively, the risk scores from Formula 20 and Formula 22 can be first dichotomized into risk factors as:

RF1=1 if RS1>0.408, and RF1=0 if RS1<=0.408

RF2=1 if RS2>0.436, and RF2=0 if RS2<=0.436

RF=RF1+RF2

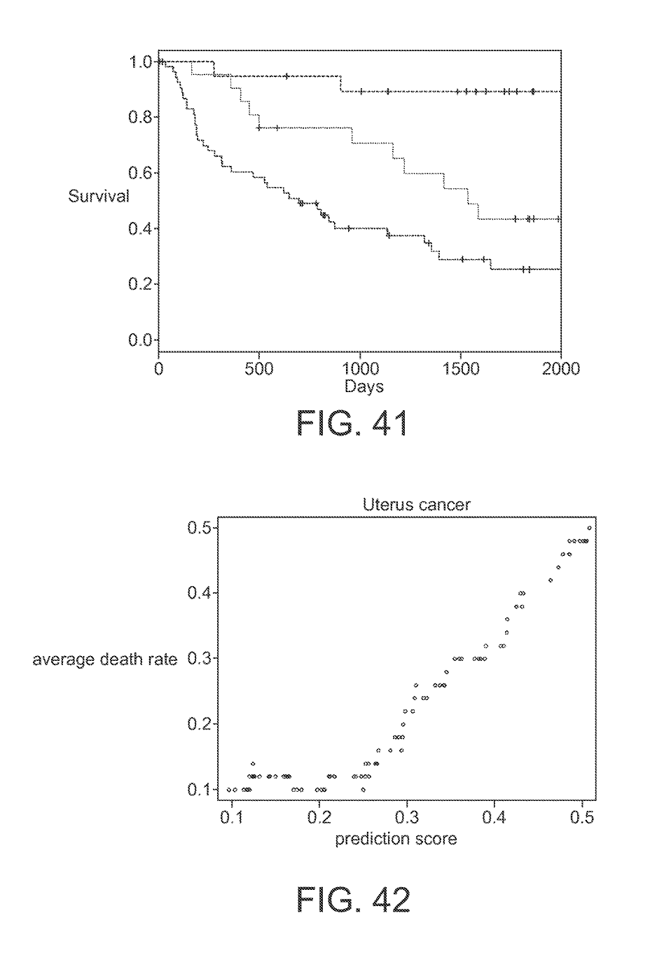

FIG. 41 shows the Kaplan-Meier plot for patients with RF ranges from 0 to 2. The Chi-square for 2 degrees of freedom is 19.6 (P=5.7×10⁻⁵).

TABLE 40
Prognosis signature component 1 (anti-correlated with poor outcome)

| probe | Gene |
|---|---|
| merck-NM_015208_at | ANKRD12 |
| merck-NM_005410_s_at | SEPP1 CCDC152 |
| merck-NM_013262_s_at | MYLIP |
| merck-NM_012096_at | APPL1 |
| merck-AK057337_at | LINC00924 |
| merck-AK091904_at | — |
| merck-NM_000867_at | HTR2B |
| merck2-BX647414_a_at | — |
| merck-NM_014774_at | EFCAB14 |
| merck-NM_003022_at | SH3BGRL |
| merck-BX647414_s_at | — |
| merck2-CN371999_a_at | FBXL3 |
| merck2-AA155774_at | RHOJ |
| merck-AV703096_s_at | — |
| merck-NM_031474_at | NRIP2 |
| merck-AK022074_a_at | RUFY3 |
| merck-NM_012158_at | FBXL3 |
| merck2-CN308012_at | EFCAB14 |
| merck2-NM_003922_at | HERC1 |
| merck-ENST00000375110_at | EPC1 |
| merck2-ENST00000367436_a_at | CDC73 |
| merck-BX647696_a_at | TACC1 |
| merck-BC036296_at | — |
| merck-BF663662_at | — |
| merck-AK022059_at | SNX18 |
| merck-AK092045_s_at | CCDC50 |
| merck-ENST00000368886_at | IKZF5 |
| merck-NM_194434_at | VAPA |
| merck2-CR623081_x_at | — |
| merck2-AK223450_a_at | MPPE1 GNAL |
| merck-BX098521_at | MAF LOC101928230 |
| merck-NM_015602_a_at | TOR1AIP1 |
| merck2-DA809388_at | CCDC50 |
| merck2-NM_012158_at | FBXL3 |
| merck2-AF063564_x_at | — |
| merck2-AF063564_at | — |
| merck-AB008109_a_at | RGS5 |
| merck2-CD512895_at | MYCBP2 |
| merck2-AF030108_at | RGS5 |
| merck-ENST00000361850_at | LINC00310 |
| merck2-AI201749_x_at | AR |
| merck-NM_016089_at | ZNF589 |
| merck-NM_183419_s_at | RNF19A |
| merck-NM_003895_at | SYNJ1 |
| merck-NM_198159_at | MITF |
| merck2-AI201749_at | AR |
| merck-NM_033439_at | IL33 |
| merck-BC090936_at | ZBTB20 |
| merck2-BC013872_at | TP73-AS1 |
| merck-AF131806_at | RGS3 |
| merck-AW977864_at | — |
| merck2-CA312624_at | UQCRB |
| merck2-N95413_at | CREBL2 |
| merck-NM_017831_at | RNF125 |
| merck-CR604678_s_at | KRCC1 |
| merck2-AL049423_at | — |
| merck-AY007149_at | CEP350 |
| merck2-NM_024529_at | CDC73 |
| merck-AF147316_at | — |
| merck-BC030112_at | HIPK3 |
| merck2-AL049787_at | N4BP2L1 |
| merck-NM_002022_at | FMO4 |
| merck-NM_005449_at | FAIM3 IL24 |
| merck2-NM_021140_at | KDM6A CXorf36 |
| merck-AL834204_a_at | ANKRD12 |
| merck2-CB852612_at | SNX18 |
| merck-NM_017719_at | SNRK |
| merck-NM_015346_at | ZFYVE26 |
| merck-BC039516_s_at | — |
| merck2-NM_152267_at | RNF185 |
| merck2-NM_207292_at | MBNL1 |
| merck2-NM_031491_at | RBP5 |
| merck-NM_020940_s_at | FAM160B1 |
| merck2-BG701526_at | — |
| merck-NM_000109_at | DMD |
| merck-BX648284_s_at | ITGA1 |
| merck2-NM_016302_at | CRBN |
| merck-NM_002697_a_at | POU2F1 |
| merck-CR595827_s_at | PNRC2 |
| merck-AK055652_at | CCDC50 |
| merck-NM_001025197_s_at | CHI3L2 |
| merck-NM_001289_at | CLIC2 |
| merck-AF086173_at | TOR1AIP1 |
| merck-NM_005149_at | TBX19 |
| merck-NM_001008390_at | CGGBP1 |
| merck-NM_032738_at | FCRLA |
| merck-AB011115_at | ZNF862 |
| merck-NM_015460_at | MYRIP |
| merck2-NM_032738_at | FCRLA |
| merck-BX648371_at | LINC00861 |
| merck-BM561378_at | ACER3 |
| merck2-DB317311_at | GIMAP1 |
| merck-NM_018105_at | THAP1 |
| merck2-AK129610_at | SH3BGRL |
| merck-AL832613_at | SLC46A1 |
| merck2-NM_023075_at | MPPE1 GNAL |
| merck2-AA551214_a_at | MBNL1 |
| merck-NM_024756_at | MMRN2 |
| merck-AK128852_a_at | — |
| merck2-NM_080416_a_at | |

TABLE 41
Prognosis signature component 2 (correlated with poor outcome)

| probe | Gene |
|---|---|
| merck-BQ919512_s_at | ALYREF |
| merck-NM_198175_s_at | NME1 |
| merck2-NM_005782_at | ALYREF |
| merck-NM_001536_at | PRMT1 |
| merck2-AI654832_a_at | ALYREF |
| merck2-NM_033362_at | MRPS12 |
| merck2-DC428989_at | HNRNPK |
| merck-NM_172341_at | PSENEN |
| merck-NM_020438_at | DOLPP1 |
| merck2-BI602361_s_at | — |
| merck2-BC002505_at | SNRPF |
| merck-CR407609_a_at | MRPS12 |
| merck-ENST00000311926_s_at | UBE2S |
| merck2-DA435913_at | NCL |
| merck-NM_003860_s_at | BANF1 |
| merck2-DA572591_a_at | NCL |
| merck-NM_005796_a_at | NUTF2 CEP112 |
| merck-NM_015179_s_at | RRP12 |
| merck-DA418198_s_at | LARP1 |
| merck-NM_052850_s_at | GADD45GIP1 |
| merck-NM_003707_s_at | RUVBL1 |
| merck-NM_001970_s_at | EIF5AL1 EIF5A |
| merck2-BX363921_x_at | TOMM22 |
| merck2-AL599091_x_at | C5orf15 |
| merck-NM_002809_at | PSMD3 |
| merck-NM_006428_at | MRPL28 |
| merck-NM_002949_at | MRPL12 |
| merck2-XM_001134348_at | ANAPC11 |
| merck-NM_003258_at | TK1 |
| merck-BI860175_a_at | COQ4 |
| merck-NM_032301_at | FBXW9 |
| merck2-BQ674733_at | NUTF2 |
| merck2-BM504304_a_at | LSM4 |
| merck-NM_016199_s_at | LSM7 |
| merck2-BM759128_a_at | DDX54 |
| merck-NM_144998_at | STRA13 ASPSCR1 |
| merck-BC025772_s_at | EHMT1 |
| merck-NM_002720_at | PPP4C |
| merck-NM_015679_at | TRUB2 |
| merck-ENST00000322030_x_at | SET |
| merck2-EF036485_at | — |
| merck-NM_177542_at | SNRPD2 |
| merck-CR594938_s_at | RRP1 |
| merck2-AI809856_at | RPL27A |
| merck-BG771720_a_at | EMC8 |
| merck-NM_001002031_s_at | ATP5G2 |
| merck-CB995181_a_at | LSM4 |
| merck2-BG829700_at | — |
| merck-NM_016034_at | MRPS2 |
| merck-NM_001833_at | CLTA |
| merck-NM_006114_s_at | TOMM40 APOE |
| merck-NM_032353_at | VPS25 WNK4 |
| merck2-CB122391_x_at | — |
| merck-ENST00000306014_a_at | DDX54 |
| merck2-EF534308_x_at | — |
| merck2-BG822880_x_at | — |
| merck-CA866470_a_at | RAD23B |
| merck-NM_006808_at | SEC61B |
| merck-NM_017503_at | SURF2 |
| merck-BC066298_a_at | LSM12 |
| merck-CR596106_a_at | CNPY2 |
| merck-ENST00000355703_s_at | PCNXL3 |
| merck-ENST00000376263_a_at | HNRNPK |
| merck-AK057925_at | CDKN2AIPNL |
| merck2-NM_001040161_x _at | C16orf13 |
| merck2-CN304837_at | PFDN2 |
| merck-BC000118_at | CLTA |
| merck2-DB483456_at | YWHAG |
| merck2-CA848513_at | CALR |
| merck-AI911220_s_at | VPS4A |
| merck-NM_004870_at | MPDU1 |
| merck2-U28936_s_at | — |
| merck-BC036909_at | LOC284889 MIF |
| merck-NM_025233_at | COASY |
| merck2-BC065000_a_at | TCEB2 |
| merck2-CD579847_at | CALR |
| merck2-AU132133_at | UBE2Q2 |
| merck-NM_006221_at | PIN1 |
| merck-AY735339_s_at | CSNK2A1 CSNK2A3 |
| merck-BM555073_s_at | SNHG16 |
| merck2-NM_003096_at | SNRPG |
| merck-ENST00000372692_s_at | SET PARD3 |
| merck-NM_006356_a_at | ATP5H RAP1B |
| merck2-CB122391_at | — |
| merck2-BM755263_a_at | YWHAE |
| merck-NM_000990_x_at | RPL27A |
| merck2-BG748146_a_at | FXN |
| merck-NM_152383_s_at | DIS3L2 |
| merck-NM_006666_at | RUVBL2 |
| merck2-DA643319_at | EHMT1 |
| merck-NM_002904_a_at | NELFE CFB |
| merck2-NM_016050_a_at | MRPL11 |
| merck-NM_003310_at | TSSC1 LOC101927554 |
| merck-NM_006579_at | EBP TBC1D25 |
| merck-NM_014047_at | C19orf53 |
| merck2-BU623044_at | ERCC2 |
| merck-NM_175614_at | NDUFA11 |
| merck-BP224564_a_at | YY1 |
| merck-XM_939690_at | RPS15P9 |
| merck2-AA081397_x_at | — |

TABLE 44
Proliferation signature

| probe | Gene |
|---|---|
| merck-NM_003318_at | TTK |
| merck-NM_014791_at | MELK |
| merck-NM_001786_a_at | CDK1 RHOBTB1 |
| merck-NM_001790_at | CDC25C |
| merck-NM_014176_at | UBE2T |
| merck-BF511624_s_at | BUB1B |
| merck-NM_005030_at | PLK1 |
| merck-NM_181802_at | UBE2C |
| merck-NM_004217_at | AURKB |
| merck-NM_201567_at | CDC25A |
| merck-NM_198436_s_at | AURKA |
| merck-NM_001255_s_at | CDC20 |
| merck-NM_003579_at | RAD54L |
| merck-NM_004336_at | BUB1 RGPD6 |
| merck-NM_031299_at | CDCA3 GNB3 |
| merck-NM_004237_at | TRIP13 |
| merck-BC001459_s_at | RAD51 |
| merck-NM_012484_at | HMMR |
| merck-AB042719_a_at | MCM10 |
| merck-NM_018518_at | MCM10 |
| merck-NM_012291_at | ESPL1 PFDN5 |
| merck-NM_014750_at | DLGAP5 |
| merck-NM_199413_at | PRC1 |
| merck-NM_130398_at | EXO1 |
| merck-NM_199420_s_at | POLQ |
| merck-NM_005733_at | KIF20A CDC23 |
| merck-NM_004856_at | KIF23 |
| merck-NM_004701_at | CCNB2 |
| merck-NM_014321_at | ORC6 |
| merck-NM_002466_at | MYBL2 |
| merck-NM_030919_at | FAM83D |
| merck-NM_003504_at | CDC45 |
| merck-BC075828_a_at | GTSE1 |
| merck-NM_016426_at | GTSE1 TRMU |
| merck-NM_001012409_at | SGOL1 |
| merck-NM_018136_s_at | ASPM |
| merck-NM_018685_at | ANLN |
| merck-NM_012112_at | TPX2 |
| merck-NM_018101_at | CDCA8 |
| merck-NM_001237_a_at | CCNA2 EXOSC9 |
| merck-NM_018454_at | NUSAP1 |
| merck-NM_001211_at | BUB1B |
| merck-U63743_a_at | KIF2C |
| merck-CRS96700_a_at | RRM2 |
| merck-NM_012310_at | KIF4A GDPD2 |
| merck-NM_013277_a_at | RACGAP1 |
| merck-NM_018154_at | ASF1B PRKACA |
| merck-BC024211_a_at | NCAPH |
| merck-NM_152515_at | CKAP2L |
| merck-NM_018131_at | CEP55 |
| merck-NM_002417_at | MKI67 |
| merck-CR607300_a_at | MKI67 |
| merck-BI868409_a_at | MKI67 |
| merck-NM_001813_at | CENPE |
| merck-CR602926_s_at | CCNB1 |
| merck-NM_001809_at | CENPA |
| merck-NM_080668_at | CDCA5 |
| merck-AK223428_a_at | BIRC5 |
| merck-NM_005480_at | TROAP |
| merck-NM_021953_at | FOXM1 |
| merck-NM_144508_at | CASC5 |
| merck-NM_019013_at | FAM64A PITPNM3 |
| merck-hCT1776373.2_s_at | DEPDC1 OTUD7A |
| merck-NM_004091_at | E2F2 |
| merck-NM_004219_x_at | PTTG1 |
| merck-NM_002263_a_at | KIFC1 |
| merck-AF331796_a_at | NCAPG |
| merck-NM_145060_at | SKA1 |
| merck-BC048988_a_at | SKA3 |
| merck-NM_152259_s_at | TICRR KIF7 |
| merck-ENST00000243201_a_at | HJURP |
| merck-ENST00000333706_x_at | BIRC5 |
| merck-ENST00000335534_s_at | KIF18B |
| merck-AY605064_at | CLSPN |
| merck2-AK097710_at | CDC25C |
| merck2-AF043294_at | BUB1 RGPD6 |
| merck2-AU132185_at | MKI67 |
| merck2-BC098582_at | KIF14 |
| merck2-BT006759_at | KIF2C |
| merck2-BC006325_at | GTSE1 TRMU |
| merck2-BC006325_x_at | GTSE1 TRMU |
| merck2-AL832036_at | CKAP2L |
| merck2-DQ890621_at | CDC45 |
| merck2-NM_005196_at | CENPF |
| merck2-AV714642_at | ANLN |
| merck2-BC034607_at | ASPM |
| merck2-BC001651_at | CDCA8 |
| merck2-AF098158_at | TPX2 |
| merck2-NM_001168_at | BIRC5 |
| merck2-AK023483_at | NUSAP1 |
| merck2-NM_145061_at | SKA3 |
| merck2-NM_018410_at | HJURP |
| merck2-AL517462_s_at | — |
| merck2-ENST00000333706_s_at | — |
| merck2-BX648516_at | SGOL1 |
| merck2-AK000490_a_at | DEPDC1 |
| merck2-ENST00000370966_a_at | DEPDC1 OTUD7A |
| merck2-AB046790_at | CASC5 |
| merck2-CR936650_at | ANLN |
| merck2-AL519719_a_at | BIRC5 |
| merck2-NM_145060_a_at | SKA1 |
| merck2-NM_001039535_a_at | SKA1 |

### Example 11

### Prognostic Model for Uterus

This example describes a uterus prognosis model based on gene expression profiling data. The model contains two gene expression signatures as components. In the second part of the example, the number of genes in each signature is reduced to 10 genes to simplify the implementation of this prognosis model.

A total of 342 samples were profiled by Affymetrix® expression arrays. A composite model was built using the first half of samples and the model validated using the second half of samples. In the first half of samples, 168 samples had outcome data (alive or dead). Among them, 119 had good outcome and 49 had poor outcome. One good outcome patient did not have stage data. In the second half of samples, all 171 had outcome data. Among 130 good outcome patients, 13 did not have stage data. In the 41 poor outcome patients, 5 did not have stage data.

Two groups of genes (100 Affymetrix® probe-sets each) were identified in 168 training samples which are either correlated or anti-correlated with poor outcome. These two groups of genes are displayed in Tables 47 & 48.

A model was built in the training set using a general linear model (from the R package) using the following equation:

Uterus Cancer Risk Score=0.33692+0.10294*(prg2−prg1)+0.09746*stage (Formula 24),

where “prg1” is a score calculated from prognosis genes in Table 47 and “prg2” is a score calculated from prognosis genes in Table 48. The scores can be calculated by averaging the log2(intensity) of each probe in the geneset.

The performance of this model is evaluated in reserved validation set of 153 samples with also the stage data. FIG. 42 shows the predicted death rate vs. the actual average (running average of 50 samples as ranked by the prediction score) death rate. As shown in the Figure, the model predicts the average death rate very well.

The detailed information about number of samples, number of deaths, and the death rate in each prediction score bin are summarized in Table 49.

TABLE 49
Average death rate versus prediction score.

| Score | Number of samples | Number of death | Death Rate |
|---|---|---|---|
| <0.2 | 61 | 5 | 0.082 |
| 0.2-0.4 | 46 | 7 | 0.152 |
| 0.4-0.6 | 32 | 15 | 0.469 |
| >0.6 | 14 | 9 | 0.643 |

Using a threshold of 0.4, the odds ratio for overall survival is 9.3, 95% CI: 3.8 -22.5, Fisher's Exact Test p-value=1.1×10⁻⁷.

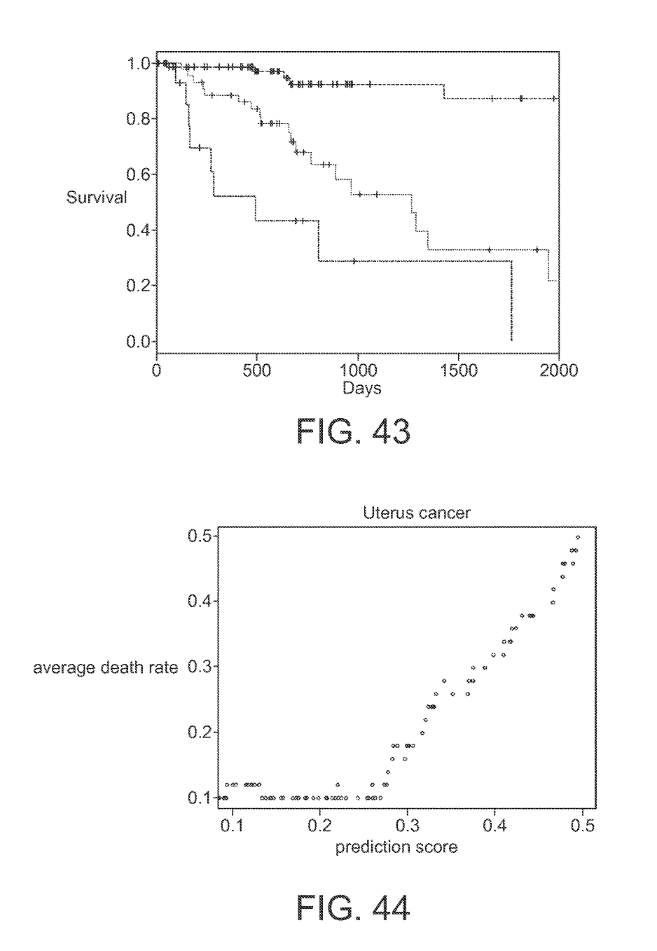

Patients can be further divided into good (risk score<0.32), medium (score 0.32-0.6) and poor (score>0.6) prognosis groups. FIG. 43 shows the Kaplan-Meier curves for these 3 groups. The Chi-square on 2 degrees of freedom is 40 (P=2.1×10⁻⁹).

The number of genes in each pathway was reduced to 10 genes.

Prognosis signature component 1 (prg1):
Probe IDs: merck-ENST00000369936_at, merck-NM_004058_at, merck-NM_002407_at, merck-AI918006_at, merck2-AK025905_at, merck-NM_145051_s_at, merck2-DT217746_at, merck-NM_152376_s_at, merck-NM_006551_at, merck2-CA489714 at
Gene symbols: KIAA1324, CAPS, SCGB2A1, UBX1V10, SOX17, RNF183, ASRGL1, UBXN10, SCGB1D2, SPDEF

Prognosis Signature Component 2 (prg2):
Probe IDs: merck2-BM904739_at, merck-NM_153485_at, merck-NM_003875_at, merck-NM_000540_at, merck-NM_021922_at, merck-NM_181573_s_at, merck-ENST 00000311926_s_at, merck2-BC112898_at, merck-NM_007274_s_at, merck-NM_004181_at
Gene symbols: MRGBP, NUP155, GMPS, RYR1, FANCE, RFC4, UBE2S, ZNF623, ACOT7, UCHL1

The scores derived from these 10-genes are correlated to the original scores at the level of 0.97 for prg1, 0.94 for prg2.

Using the reduced gene sets, the updated predictive model is:

Uterus Cancer Risk Score=0.15030+0.06071*(prg2−prg1)+0.10849*stage (Formula 25).

Note, the exact coefficients will change depending on the final selection of the technology platform (RNAseq vs. arrays, PCR), and the probe sets or gene lists.

FIG. 44 shows the predicted death rate vs. the actual average (running average of 50 samples as ranked by the prediction score) death rate for this updated model. As shown in the Figure, the model predicts the average death rate very well.

The detailed information about number of samples, number of deaths, and the death rate in each prediction score bin are summarized in Table 50.

TABLE 50
Average death rate versus prediction score.

| | Number os | Number of | |
|---|---|---|---|
| Score | samples | death | Death Rate |
| <0.2 | 63 | 6 | 0.095 |
| 0.2-0.4 | 44 | 7 | 0.159 |
| 0.4-0.6 | 34 | 14 | 0.412 |
| >0.6 | 12 | 9 | 0.750 |

Using a threshold of 0.32, the odds ratio for overall survival is 8.5 (95% CI: 3.5-20.6), Fisher's Exact Test p-value=4.1×10⁻⁷.

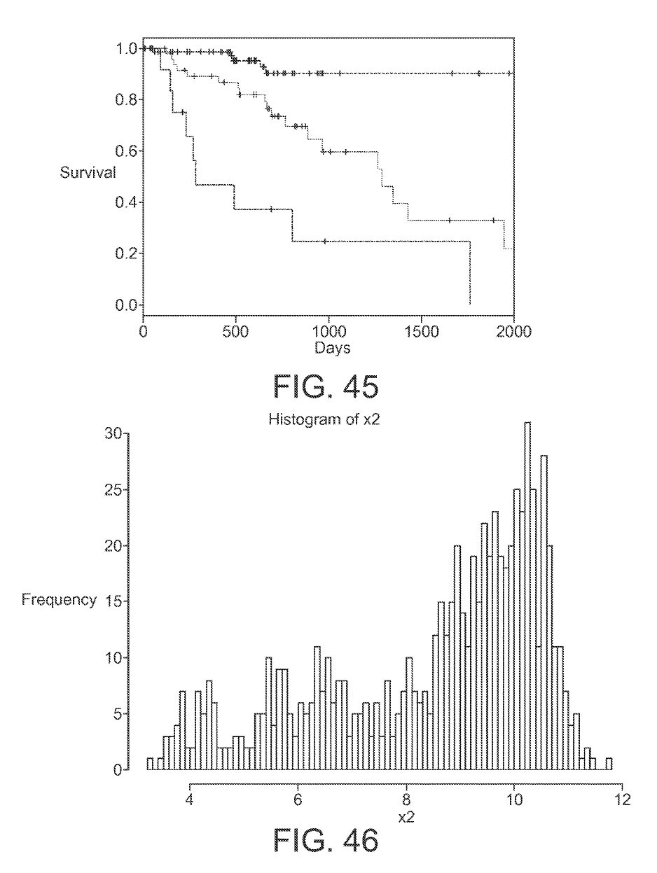

Patients can be further divided into good (risk score<0.32), medium (score 0.32-0.6) and poor (score>0.6) prognosis groups. FIG. 45 shows the Kaplan-Meier curves for these 3 groups. The Chi-square on 2 degrees of freedom is 40.9 (P=1.3×10⁻⁹).

TABLE 47
Prognosis signature component 1 (anti-
correlated with poor outcome)

| Probe | Gene |
|---|---|
| merck-AL040975_at | ESR1 |
| merck-NM_005397_at | PODXL MKLN1 |
| merck-AI918006_at | UBXN10 |
| merck-AL137566_at | PGR |
| merck-NM_022454_at | SOX17 |
| merck2-AA148029_at | PODXL MKLN1 |
| merck2-AK025905_at | SOX17 |
| merck-NM_002407_at | SCGB2A1 |
| merck-NM_001012993_at | C9orf152 |
| merck2-NM_000125_at | ESR1 |
| merck-NM_000125_at | ESR1 |
| merck-NM_018728_at | MYO5C |
| merck2-AL050116_at | ESR1 |
| merck-AF016381_a_at | PGR |
| merck-BX106921_at | PGR |
| merck-NM_006551_at | SCGB1D2 |
| merck-BX648070_at | C2orf88 HIBCH |
| merck-ENST00000369936_at | KIAA1324 |
| merck-NM_152376_s_at | UBXN10 |
| merck-NM_014178_s_at | STXBP6 |
| merck2-BX648631_at | UBXN10 |
| merck-BC028018_at | LOC100129098 |
| merck2-BQ684833_at | ACSL5 |
| merck-NM_014211_at | GABRP |
| merck-NM_021069_at | SORBS2 |
| merck-BC011052_a_at | TRIM2 |
| merck-AL834346_at | STXBP6 |
| merck-ENST00000347491_s_at | ESR1 |
| merck2-DT217746_at | ASRGL1 |
| merck-NM_004058_at | CAPS |
| merck-NM_025080_s_at | ASRGL1 |
| merck-NM_005080_at | XBP1 |
| merck-NM_018414_at | ST6GALNAC1 |
| merck-NM_020775_s_at | KIAA1324 |
| merck2-AM392558_at | SORBS2 |
| merck-ENST00000319471_a_at | SORBS2 |
| merck2-NM_021777_at | ADAM28 |
| merck-NM_015541_s_at | LRIG1 |
| merck-ENST00000285039_at | MYO5B |
| merck-NM_002644_s_at | PIGR |
| merck2-CB852618_at | GRAMD3 |
| merck2-NM_016930_at | STX18 |
| merck-BC017958_at | CCDC160 |
| merck-NM_013992_at | PAX8 |
| merck-NM_174921_at | SMIM14 |
| merck-NM_003212_at | TDGF1 |
| merck2-CA489714_at | SPDEF |
| merck2-BG742453_a_at | PAM |
| merck-AJ420553_at | ID4 |
| merck-NM_138766_s_at | PAM |
| merck2-AF137334_at | ADAM28 |
| merck-NM_001669_at | ARSD |
| merck2-NM_014133_at | SORBS2 |
| merck-NM_175887_at | PRR15 |
| merck-NM_018050_at | MANSC1 |
| merck2-CB241906_at | ST6GALNAC1 |
| merck-ENST00000369949_s_at | C1orf194 |
| merck-AL702564_at | PGR |
| merck-NM_001025593_at | ARFIP1 |
| merck-NM_018043_at | ANO1 |
| merck-NM_012391_at | SPDEF |
| merck-NM_021785_at | RAI2 |
| merck-NM_014265_at | ADAM28 |
| merck2-BC008590_at | GRAMD3 |
| merck2-CB962832_at | ID4 |
| merck-NM_003774_at | POC1B-GALNT4 GALNT4 |
| merck-NM_015271_at | TRIM2 |
| merck-AK128437_a_at | GALNT7 |
| merck2-BM695584_at | ARHGAP26 |
| merck-NM_001004303_at | C1orf168 |
| merck-BC094795_a_at | PIK3R1 |
| merck-NM_015071_at | ARHGAP26 |
| merck-NM_145051_s_at | RNF183 |
| merck-NM_001915_at | CYB561 |
| merck-AW970730_at | ST6GALNAC1 |
| merck-BC002976_s_at | CYB561 |
| merck-NM_015198_at | COBL |
| merck-CA427248_at | CCDC122 |
| merck-NM_001490_at | GCNT1 |
| merck-NM_022783_at | DEPTOR |
| merck2-AK026697_at | CDS1 |
| merck-NM_020879_s_at | CCDC146 |
| merck-NM_001040001_at | MLLT4 KIF25 |
| merck-NM_032321_a_at | C2orf88 |
| merck2-NM_033087_at | ALG2 |
| merck-NM_001006615_s_at | WDR31 |
| merck-NM_030630_s_at | HID1 |
| merck-NM_153000_at | APCDD1 |
| merck-NM_176813_at | AGR3 |
| merck-CR749204_s_at | PTPN3 |
| merck-NM_000266_at | NDP |
| merck-NM_004727_s_at | SLC24A1 |
| merck2-BC012630_at | SLC24A1 |
| merck-NM_015993_at | PLLP |
| merck-BC068555_a_at | ARHGAP26 |
| merck-T68445_a_at | AR |
| merck-NM_001002912_s_at | C1orf173 |
| merck2-AK023916_at | DEPTOR |
| merck-AB032983_at | PPM1H |
| merck-AK075059_at | GLIS3 |

TABLE 48
Prognosis signature component 2 (correlated with poor outcome)

| Probe | Gene |
|---|---|
| merck2-AB071393_a_at | TTL |
| merck2-AK127448_at | B4GALNT1 |
| merck2-NM_153712_at | TTL |
| merck-NM_001010911_at | CASC10 |
| merck2-BM904739_at | MRGBP |
| merck-NM_000540_at | RYR1 |
| merck-NM_006442_s_at | DRAP1 |
| merck2-AK222554_x_at | SF3A3 |
| merck-BU594972_a_at | TSC1 |
| merck-CR599730_a_at | TTL |
| merck2-BU620949_at | DRAP1 |
| merck2-AK222554_at | SF3A3 |
| merck-BC029828_at | B4GALNT1 |
| merck-NM_003875_at | GMPS |
| merck-ENST00000222607_at | STEAP1B |
| merck-NM_006143_at | GPR19 |
| merck2-BC112898_at | ZNF623 |
| merck-NM_021922_at | FANCE |
| merck2-BI602361_s_at | — |
| merck-AL832168_at | — |
| merck2-AI825916_at | TSC1 |
| merck2-BC041955_at | — |
| merck2-NM_199427_at | ZFP64 |
| merck2-AI149996_at | ADRM1 |
| merck-NM_004181_at | UCHL1 |
| merck-NM_181573_s_at | RFC4 |
| merck-BC028609_a_at | CCDC93 |
| merck-AF368281_a_at | SGTB |
| merck-ENST00000311926_s_at | UBE2S |
| merck-NM_021158_at | TRIB3 |
| merck-NM_006087_at | TUBB4A |
| merck2-AK026140_at | — |
| merck2-AK130014_at | SHC1 |
| merck-NM_003610_at | RAE1 |
| merck-NM_018270_at | MRGBP |
| merck-NM_016447_at | MPP6 |
| merck-NM_182627_at | WDR53 |
| merck-AL713706_at | DPYSL5 |
| merck-NM_014696_s_at | GPRIN2 |
| merck-AB015342_a_at | ZNF318 |
| merck2-ENST00000356433_at | DLL3 |
| merck2-BF739910_at | RBM33 |
| merck-NM_004341_at | CAD |
| merck-ENST00000313019_s_at | SHOX2 |
| merck-BC003580_s_at | CIAO1 |
| merck-NM_001426_at | EN1 |
| merck-NM_002503_at | NFKBIB |
| merck-NM_016625_s_at | RSRC1 |
| merck2-DA447204_at | SHOX2 |
| merck-AF533230_x_at | USP32 |
| merck-NM_013409_at | FST |
| merck2-BC012379_at | ZHX1-C8ORF76 |
| merck-NM_007274_s_at | ACOT7 |
| merck-AK123535_at | FBXL18 |
| merck-NM_152699_s_at | SENP5 |
| merck-NM_007002_at | ADRM1 |
| merck2-BC025263_at | CDCA4 |
| merck-NM_006553_at | SLMO1 |
| merck-NM_206831_a_at | DPH3 OXNAD1 RFTN1 |
| merck-NM_006818_at | MLLT11 |
| merck-NM_000523_at | HOXD13 |
| merck-AK025697_at | FBXO45 |
| merck2-BX340398_at | SMIM13 |
| merck-AW821325_at | RAE1 |
| merck2-BC001395_at | CIAO1 |
| merck-BT009760_s_at | ZFP64 |
| merck-NM_000022_at | ADA |
| merck-DW451489_s_at | MED8 |
| merck2-NM_001017406_at | S100PBP |
| merck-ENST00000343379_a_at | SS18L1 |
| merck2-BC051770_a_at | ACTN2 |
| merck-AK129880_a_at | UBXN7 |
| merck-BC064390_a_at | HAUS5 |
| merck-NM_001039617_at | ZDHHC19 |
| merck2-NM_145733_at | 3-Sep |
| merck-BC068057_a_at | YRDC |
| merck2-NM_023008_at | KRI1 |
| merck2-BC040609_at | SENP2 |
| merck2-AB053301_at | TMEM237 |
| merck-NM_007027_at | TOPBP1 |
| merck-NM_001008949_at | ITPRIPL1 |
| merck-NM_178830_at | C19orf47 |
| merck-NM_183001_a_at | SHC1 |
| merck-AF151697_a_at | SENP2 |
| merck-ENST00000362037_at | LOC645195 |
| merck-NM_012318_at | LETM1 |
| merck-NM_153485_at | NUP155 |
| merck-NM_002808_at | PSMD2 |
| merck-BC047330_at | MPP6 |
| merck-NM_024333_at | FSD1 STAP2 |
| merck-NM_152363_at | ANKLE1 |
| merck-AK126101_a_at | PLXNA1 |
| merck2-AB209521_at | ACTN2 |
| merck-NM_015327_at | SMG5 PTS |
| merck2-BM674474_at | — |
| merck-BC014211_x_at | TCEA2 |
| merck-NM_024721_a_at | ZFHX4 |
| merck-BC042486_a_at | KIF3C |
| merck-NM_203486_s_at | DLL3 |
| merck-NM_001350_s_at | DAXX |

### Example 12

### Prognostic Model for Ovarian Cancer

This example describes an ovarian cancer prognosis model based on gene expression profiling data. The model contains two gene expression signatures as components. In the second part of the example, the number of genes in each signature is reduced to 10 genes to simplify the implementation of this prognosis model. Since both the prognosis signatures derived from the current dataset and the pre-defined proliferation signature predict patient outcome, both predictors were combined.

A total of 731 samples were profiled by Affymetrix® expression arrays. Among them 362 were alive and 367 were dead (2 with status unknown) at the time of data collection. Samples were equally divided into training (365 samples) and validation (366 samples) set. In the training set, patients were first divided into two groups based on genome-wide 2-D clustering, and the markers associated with these two groups were identified. Among the markers correlated with group IDs, one group of markers (X2) led to successful prognosis biomarker identification when used in the patient stratification.

In the training set, a 2D-clustering based on 3171 highly variable genes (standard deviation of log2 intensity)>1.5) was performed, and patients were partitioned into two groups. Genes were then selected that are highly variable (std(log2 intensity)>2) and with correlation to the group ID greater than 0.5 (positive- and negative-correlation). Each group of genes was used to stratify patients for prognosis, and a group of genes (listed in Table 51) enabled discovery of strong prognosis patterns in the training set.

TABLE 51
patient stratification markers

| | | Correlation to |
|---|---|---|
| Probe ID | Gene | group ID |
| merck-AI732822_at | KCND2 | 0.523155 |
| merck2-AI264554_at | — | 0.543379 |
| merck-BX103595_at | — | 0.580491 |
| merck-NM_015507_at | EGFL6 | 0.541111 |
| merck-NM_001878_at | CRABP2 | 0.526755 |
| merck-NM_012427_at | KLK5 | 0.54748 |
| merck-NM_005046_s_at | KLK7 | 0.554217 |
| merck-NM_016725_s_at | FOLR1 | 0.502639 |
| merck-NM_001276_at | CHI3L1 | 0.506725 |
| merck-ENST00000373692_a_at | PTGS1 | 0.582718 |

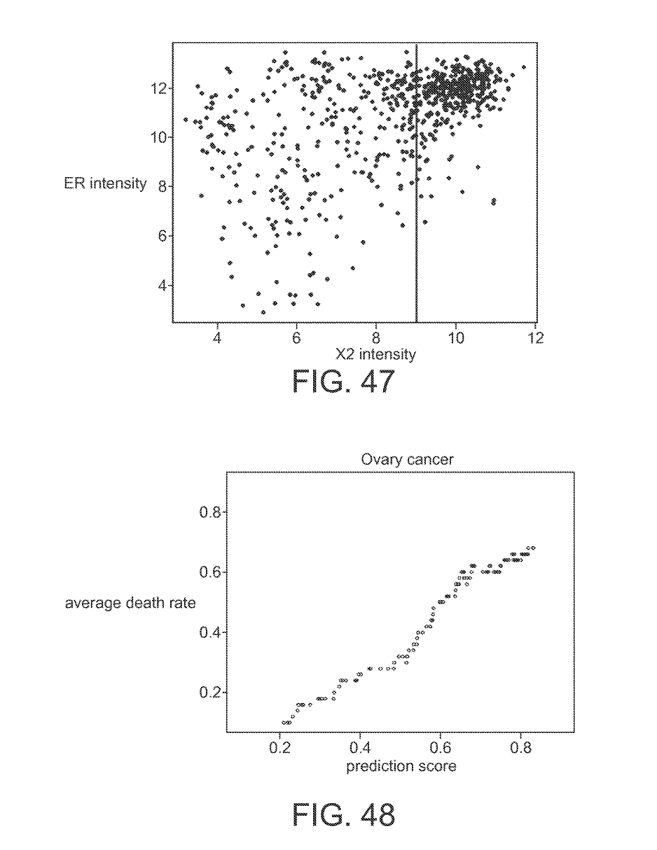

Patient stratification was based on the average log2 intensity from the probes listed in Table 51. FIG. 46 shows the histogram of the X2 probe intensities in ovarian cancer. There is peak around log2 intensity of 10, and a uniform distribution below the intensity peak. When the X2 intensity versus the estrogen-receptor level was checked, almost all the patients with high X2 intensity also had uniformly high ER intensity, contrasting to the low-X2 patients where ER levels had wide range (FIG. 47). A threshold was therefore placed at X2 =9. Patients with X2>9 and X2<9 will be termed X2+ and X2− in the rest of the example.

In the training set with 365 samples, 175 patients had X2− (X2<9), and 190 patients with X2+ (X2>9). In the X2−, 174 patients had outcome data, 88 were dead at the time of data collection. In the X2+ patients, 189 had outcome data, 118 were dead. Prognosis signature discovery was tried for both X2− and X2+ populations. For this example, the focus is on X2− since it yielded a more significant prognostic model.

In the validation set with 366 samples, 170 patients are X2− and 196 patients are X2+. The poor outcome patients (dead at the last time of data collection) are 75 and 86 respectively.

Patients with high X2 had slightly higher poor outcome rate, but X2 itself is not a strong prognosis factor.

Two groups of genes (100 Affymetrix® probe-sets each) were identified in 174 X2− raining samples which are either correlated or anti-correlated with poor outcome. These two groups of genes are displayed in Tables 52 & 53.

A model was built in the X2− training set using a general linear model (from the R package) using the following equation:

Ovarian Cancer Risk Score=−0.01678−(0.09271*prg1)+(0.10882*prg2)+(0.17827*stage) (Formula 26),

where “prg1” is a score calculated from prognosis genes in Table 52 and “prg2” is a score calculated from prognosis genes in Table 53, and the stage is the composite stage. The scores can be calculated by averaging the log2(intensity) of each probe in the geneset.

The performance of this model is evaluated in reserved validation set of 170 X2− samples. FIG. 48 shows the predicted death rate vs. the actual average (running average of 50 samples as ranked by the prediction score) death rate. As shown in the Figure, the model predicts the average death rate very well.

The detailed information about number of samples, number of deaths, and the death rate in each prediction score bin are summarized in Table 54.

TABLE 54
Average death rate versus prediction score.

| | Number of | Number of | |
|---|---|---|---|
| Score | samples | death | Death Rate |
| <0.2 | 23 | 0 | 0.000 |
| 0.2-0.4 | 25 | 4 | 0.160 |
| 0.4-0.6 | 27 | 11 | 0.407 |
| 0.6-0.08 | 50 | 30 | 0.600 |
| >0.8 | 35 | 27 | 0.771 |

Using a threshold of 0.5, the odds ratio for overall survival is 9.6 (95% CI: 4.1-22.4), Fisher's Exact Test p-value=6.2×10⁻⁹.

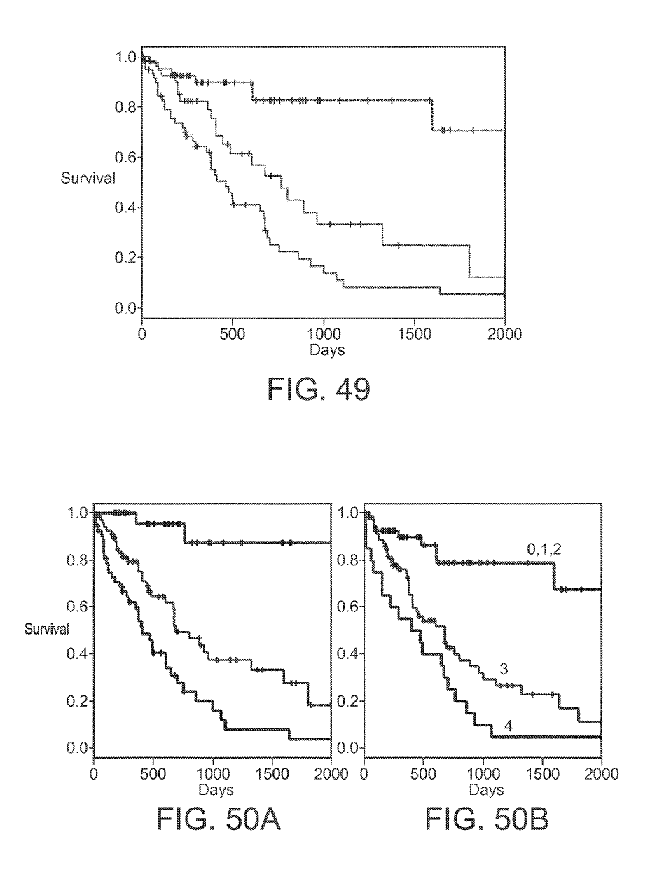

Patients can be further divided into good (risk score<0.5), medium (score 0.5-0.7) and poor (score>0.7) prognosis groups. FIG. 49 shows the Kaplan-Meier curves for these 3 groups. The Chi-square on 2 degrees of freedom is 34.3 (P=3.6×10⁻⁸).

In the prognosis model, two components are based signatures, and one component based on tumor stage. The signatures and tumor stage had similar prognosis power in the validation set. FIGS. 50A and 50B shows the prediction based on the signature only (using Formula 26 but drop the stage component) and tumor stage only. The predictive powers are very similar (Chi-squares on 2 degree of freedom are 34 for the signatures and 27.9 for the tumor stage).

The number of genes in each signature can be reduced to 10 genes.

Prognosis Signature Component 1 (prg1):
Probe IDs: merck-NM_025145_at, merck-AB051484_at, merck-NM_018430_s_at, merck-NM_018897_at, merck-NM_145170_at, merck-NM_181643_at, merck-NM_031421_at, merck-NM_003551_at, merck-NM_024763_at, merck-NM_178452_s_at
Gene symbols: WDR96, DNAH6, TSNAXIP1, DNAH7, TTC18, PIFO, TTC25, NME5, WDR78, DNAAF1

Prognosis Signature Component 2 (prg2):
Probe IDs: merck-NM_021972_at, merck2-BQ002341_at, merck2-NM_007115_at, merck-NM_004460_at, merck-NM_000960_at, merck-NM_002658_at, merck-X77690_at, merck-BC007858 a_at, merck-NM_003485_at, merck-AY358331_{‥}s_{‥}at
Gene symbols: SPHK1, LINC00607, TNFAIP6, FAP, PTGIR, PLAU, TIMP3, INHBA, GPR68, NTM

The scores derived from these 10-genes are correlated to the original scores at the level of 0.96 for prg1, 0.91 for prg2.

Using the reduced gene sets, the updated predictive model is:

Ovarian Cancer Risk Score=0.26269−(0.06569*prg1)+(0.03415*prg2)+(0.18904*stage) (Formula 27).

Note, the exact coefficients will change depending on the final selection of the technology platform (RNAseq vs. arrays, PCR), and the probe sets or gene lists.

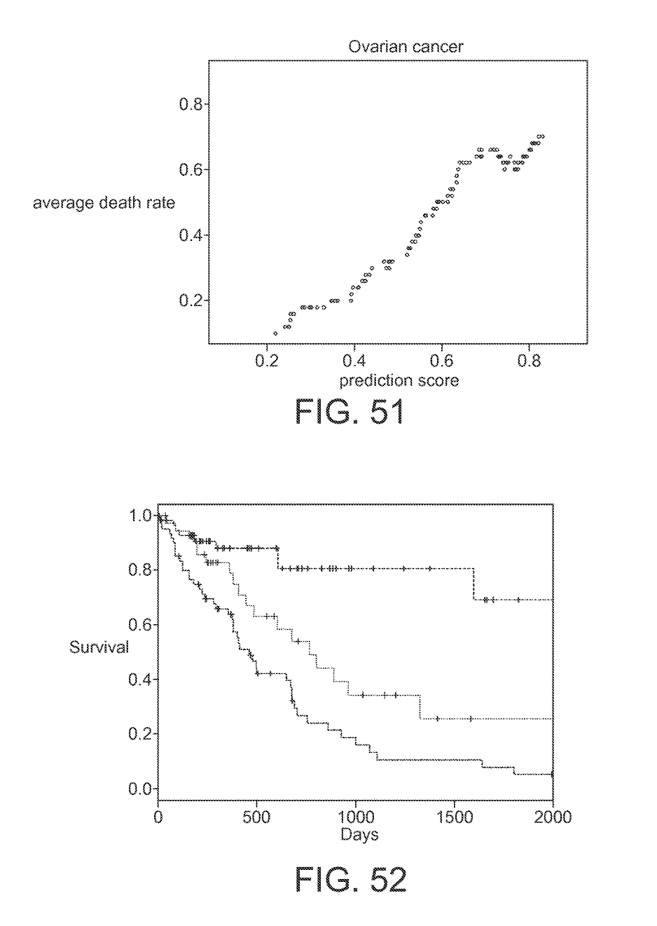

FIG. 51 shows the predicted death rate vs. the actual average (running average of 50 samples as ranked by the prediction score) death rate for this updated model. As shown in the Figure, the model predicts the average death rate very well.

Table 55 shows the detailed information about number of samples, number of deaths, and the death rate in each prediction score bin.

TABLE 55
Average death rate versus prediction score.

| | Number of | Number of | |
|---|---|---|---|
| Score | samples | death | Death Rate |
| <0.2 | 22 | 0 | 0.000 |
| 0.2-0.4 | 23 | 3 | 0.130 |
| 0.4-0.6 | 33 | 12 | 0.364 |
| 0.6-0.08 | 46 | 31 | 0.674 |
| >0.8 | 36 | 26 | 0.722 |

Using a threshold of 0.5, the odds ratio for overall survival is 9.2 (95% CI: 4.1-20.9), Fisher's Exact Test p-value=4.0×10⁻⁹.

Patients can be further divided into good (risk score<0.5), medium (score 0.5-0.7) and poor (score>0.7) prognosis groups. FIG. 52 shows the Kaplan-Meier curves for these 3 groups. The Chi-square on 2 degrees of freedom is 30.7 (P=2.1×10⁻⁷).

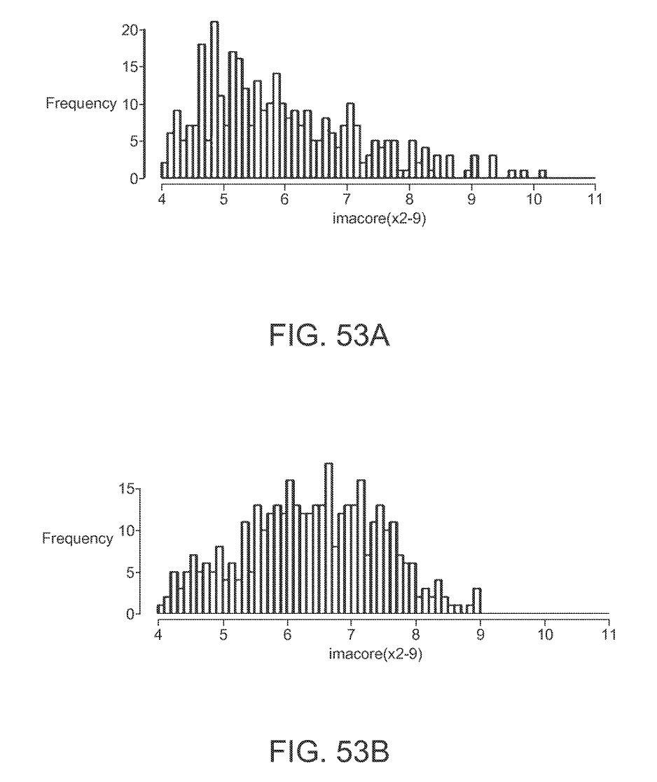

X2− and X2+ patients have different immune signature scores (FIGS. 53A and 53B), X2− patients have more spread but majority had low scores, whereas X2+ is peaked higher. When checking the outcome with immune scores, there is no relation between patient outcome and immune signature score in X2-patients, but in X2+ patients, high immune score is related to relative good outcome (P-value=1.2%).

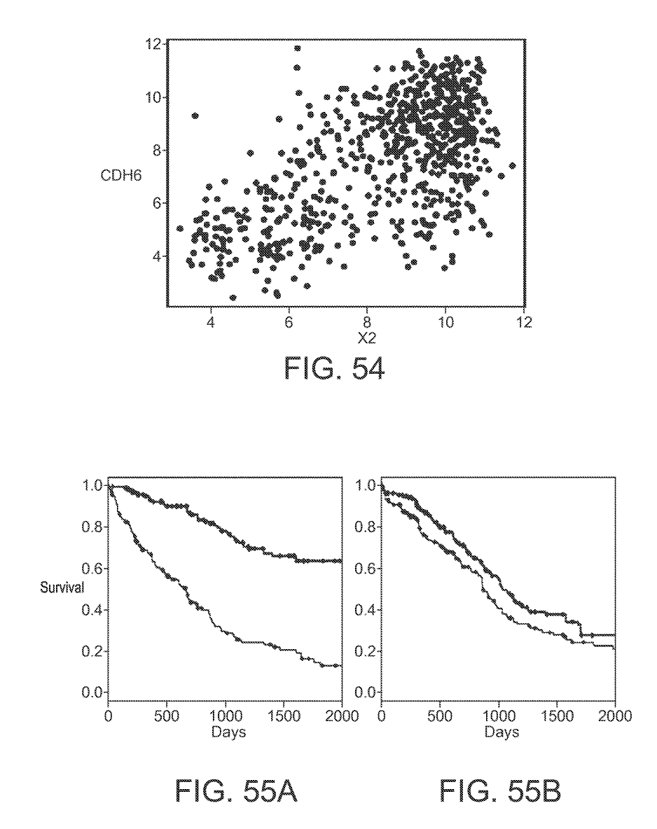

X2 is highly correlated with keratins, and cadherins, and to a certain degree, with integrins as well (FIG. 54). For example, the correlation between X2 and the average of all keratins is 0.59. Clustering based all cadherins almost perfectly segregates X2+ from X2− patients. Among the cadherins, CDH6 is correlated to X2 at 0.61. Hence, X2+ may indicate tumors were originated from more “epithelial-like” tissues.

Table 56 lists the histotype distribution between X2− ad X2+ populations. X2− is enriched for Carcinosarcoma, Clear cell adenocarcinoma, Endometroid adenocarcinoma, Granulosa cell tumor and Mucinous adenocarcinoma, whereas X2+ is enriched for Papillary serous cystadenocarcinoma and Serous cystadenocarcinoma.

TABLE 56
Number of samples in X2− and X2+ population

| | X2− | X2+ |
|---|---|---|
| Adenocarcinoma, NOS | 29 | 31 |
| Carcinoma, NOS | 15 | 27 |
| Carcinosarcoma, NOS | 8 | 0 |
| Clear cell adenocarcinoma, NOS | 21 | 0 |
| Endometrioid adenocarcinoma, NOS | 35 | 7 |
| Granulosa cell tumor, malignant | 32 | 0 |
| Mucinous adenocarcinoma | 10 | 0 |
| Papillary serous cystadenocarcinoma | 46 | 106 |
| Serous cystadenocarcinoma, NOS | 76 | 206 |
| Serous, borderline | 12 | 0 |

When the disclosed endometrium cancer prognosis signature is applied to the ovarian cancer, the performance is significantly different in X2− and X2+ populations (FIG. 55A and 55B). In X2-population, the endometrium signature is a very strong predictor (chi-square=82.5, P=0), but same model is only marginally predictive in X2+ population (chi-square=4.3, P=0.04), suggesting X2− is more “endometrium-like”.

TABLE 52
Prognosis signature component 1 (anti-
correlated with poor outcome)

| Probe | Gene |
|---|---|
| merck-NM_003551_at | NME5 |
| merck2-BC026182_at | NME5 |
| merck-NM_130897_at | DYNLRB2 LOC101928276 |
| merck-NM_003462_at | DNALI1 |
| merck-AF006386_a_at | DNALI1 |
| merck-AK055990_at | DNAH9 |
| merck-NM_145170_at | TTC18 |
| merck2-AB014543_at | CLUAP1 |
| merck2-BX093691_at | TTC18 |
| merck-ENST00000369736_a_at | PIFO |
| merck2-AI167680_a_at | CLUAP1 |
| merck-NM_018430_s_at | TSNAXIP1 |
| merck-NM_015041_a_at | CLUAP1 |
| merck-NM_152676_at | FBXO15 |
| merck-NM_181643_at | PIFO |
| merck2-XM_294004_at | RSPH4A |
| merck2-NM_001039845_at | MDH1B |
| merck-NM_031294_s_at | LRRC48 ATPAF2 |
| merck-NM_053000_s_at | EPB41L4A-AS1 |
| merck-NM_022785_s_at | EFCAB6 |
| merck-NM_145047_s_at | OSCP1 |
| merck-NM_024549_s_at | TCTN1 |
| merck-NM_014433_at | RTDR1 |
| merck2-BC034669_at | DPH5 |
| merck-AB051484_at | DNAH6 |
| merck-ENST00000341790_a_at | NME9 |
| merck-ENST00000374412_a_at | MDH1B |
| merck-G36659_at | FANK1 |
| merck-NM_001010892_at | RSPH4A |
| merck-NM_007081_s_at | RABL2A RABL2B |
| merck-NM_015958_s_at | DPH5 |
| merck2-AF546872_at | PACRG |
| merck-BC017958_at | CCDC160 |
| merck-NM_024763_at | WDR78 |
| merck2-NM_006961_at | ZNF19 |
| merck-AK027161_at | TTC12 |
| merck-NM_013249_at | ZNF214 |
| merck-NM_001551_at | IGBP1 |
| merck-NM_145235_at | FANK1 |
| merck-NM_152410_at | PACRG |
| merck2-NM_001100873_at | C16orf46 CMC2 |
| merck-NM_025145_at | WDR96 |
| merck-NM_176677_at | NHLRC4 |
| merck2-BC062574_at | NPHP1 |
| merck-NM_001008226_at | FAM154B |
| merck-U79257_at | — |
| merck-NM_032257_s_at | ZMYND12 |
| merck2-BQ576016_at | ZNF214 |
| merck-CR593886_a_at | RABL5 |
| merck2-BC043273_at | HYDIN |
| merck-BU681848_a_at | FLJ37035 LOC283038 |
| merck2-AY336746_at | NME9 |
| merck2-AK093204_at | DALRD3 WDR6 |
| merck-BX648527_at | TMEM232 |
| merck-BE044185_a_at | KIF6 |
| merck2-BU785445_at | ZMYND12 |
| merck2-NM_206837_at | OSCP1 |
| merck-BC040979_at | LINC00271 |
| merck-BX647542_s_at | PHKA1 |
| merck2-BM977387_at | — |
| merck2-CA426602_s_at | — |
| merck-NM_001031745_at | RIBC1 HSD17B10 |
| merck-ENST00000303697_at | DCDC5 |
| merck-BX571745_a_at | NPHP1 |
| merck-NM_152572_at | AK8 |
| merck2-BC029902_at | LRRC27 |
| merck-NM_022784_at | IQCH |
| merck-AL832607_s_at | SPEF2 |
| merck2-NM_000967_s_at | — |
| merck2-CA426602_at | LRRC6 |
| merck2-BC047091_a_at | ZNF19 |
| merck-BC058159_a_at | LRRC27 |
| merck-NM_024608_at | NEIL1 MAN2C1 |
| merck-NM_207417_at | C9orf171 |
| merck-NM_017775_at | TTC19 |
| merck-NM_175885_at | FAM181B |
| merck-NM_178832_s_at | MORN4 |
| merck2-AA481616_at | — |
| merck2-AK125886_at | — |
| merck-BC017993_at | SNHG8 |
| merck2-DR159121_at | FBXO21 |
| merck-NM_022777_at | RABL5 |
| merck-NM_015002_at | FBXO21 |
| merck-ENST00000341761_at | WDR31 |
| merck-NM_080667_s_at | CCDC104 |
| merck2-AL833327_at | DNAAF1 |
| merck2-AW959853_at | ATXN10 |
| merck-NM_018897_at | DNAH7 |
| merck-AL137566_at | PGR |
| merck-NM_001006615_s_at | WDR31 |
| merck2-BC007345_at | RPL13 |
| merck2-BC007345_x_at | RPL13 |
| merck-NM_004650_at | PNPLA4 |
| merck-NM_024867_s_at | SPEF2 |
| merck-NM_012119_at | CDK20 |
| merck2-AA383024_s_at | — |
| merck-NM_194270_at | MORN2 |
| merck2-BC031231_at | STK33 |
| merck2-BC033935_at | FBXO36 |
| merck-AK097547_s_at | SPEF2 |

TABLE 53
Prognosis signature component 2 (correlated with poor outcome)

| probe | Gene |
|---|---|
| merck2-AK127448_at | B4GALNT1 |
| merck-NM_021972_at | SPHK1 |
| merck-NM_003942_at | RPS6KA4 |
| merck-BC007582_a_at | CEBPG |
| merck-NM_000960_at | PTGIR |
| merck2-BQ002341_at | LINC00607 |
| merck2-NM_004145_at | MYO9B |
| merck2-BX340398_at | SMIM13 |
| merck-ENST00000332498_x_at | CYCSP3 |
| merck-NM_022338_at | C11orf24 |
| merck-X77690_at | TIMP3 |
| merck-BC005339_a_at | TPMT |
| merck-NM_004521_s_at | KIF5B |
| merck2-AK027899_a_at | RELT |
| merck2-NM_003039_at | SLC2A5 |
| merck-BC051810_a_at | RELT |
| merck-NM_138441_s_at | MB21D1 |
| merck2-D45917_a_at | TIMP3 |
| merck2-NM_007115_at | TNFAIP6 |
| merck-NM_024656_at | COLGALT1 |
| merck2-AI537528_x_at | TUBA1B |
| merck-BC071897_a_at | MCL1 |
| merck-AF006082_a_at | ACTR2 |
| merck2-AB030656_at | CORO1C |
| merck-DW451489_s_at | MED8 |
| merck-AW072050_a_at | MYO9B |
| merck-AY177688_s_at | DNAJC21 |
| merck-NM_002524_at | NRAS |
| merck-NM_054034_a_at | FN1 |
| merck-NM_002928_at | RGS16 |
| merck-NM_006884_s_at | SHOX2 |
| merck-M31164_at | TNFAIP6 |
| merck-AF143684_s_at | MYO9B |
| merck2-AF456425_a_at | DCUN1D1 |
| merck-NM_005192_at | CDKN3 |
| merck2-CA308717_at | — |
| merck-CR627287_at | ALDH1L2 |
| merck-BC073853_a_at | ACER3 |
| merck-AY171233_s_at | PTPDC1 |
| merck2-AX801509_a_at | TIMP3 |
| merck-AI160141_a_at | SLC2A5 |
| merck-NM_030759_a_at | NRBF2 |
| merck-NM_002202_at | ISL1 |
| merck2-AA661461_at | TUBA1B |
| merck2-AI566394_at | COLGALT1 |
| merck2-AA758689_at | SKIL |
| merck-NM_015459_s_at | ATL3 |
| merck2-ENST00000378047_at | FGF1 |
| merck-CR610281_a_at | TIMP3 |
| merck-NM_001189_at | NKX3-2 |
| merck-ENST00000284274_a_at | FAM105B |
| merck-81258956_a_at | PTBP3 |
| merck2-AK097588_at | ATL3 |
| merck-NM_021958_at | HLX |
| merck2-BX096261_a_at | SLC2A5 |
| merck-NM_016573_at | GMIP |
| merck-BC029828_at | B4GALNT1 |
| merck-NM_004226_at | STK17B |
| merck2-BC032912_at | NADK2 |
| merck-NM_006101_at | NDC80 |
| merck2-BM740515_at | — |
| merck-NM_014632_s_at | MICAL2 |
| merck-NM_002093_at | GSK3B |
| merck-NM_015719_at | COL5A3 |
| merck-NM_001945_at | HBEGF |
| merck2-BI824983_a_at | ACER3 |
| merck-NM_004994_at | MMP9 |
| merck-BC032697_a_at | FGF1 |
| merck2-NM_001031800_at | TIPRL |
| merck2-NM_004994_at | MMP9 |
| merck-CD106390_s_at | RAP1A |
| merck-BC006243_a_at | RGS16 |
| merck2-CR594502_at | TIMP3 |
| merck-BC035724_a_at | NAB1 |
| merck-NM_005261_at | GEM |
| merck-NM_001034173_a_at | ALDH1L2 |
| merck-NM_025217_at | ULBP2 |
| merck-NM_145805_at | ISL2 |
| merck-AJ419936_a_at | TNFAIP6 |
| merck-CR619305_a_at | GNB1 |
| merck-NM_024947_at | PHC3 |
| merck-NM_178167_a_at | ZNF598 |
| merck-NM_004460_at | FAP |
| merck2-BC028284_at | MARCKS HDAC2 |
| merck-CB529742_at | — |
| merck-NM_001009936_a_at | PHF19 |
| merck-BC087859_at | LOC401317 |
| merck-NM_018304_s_at | PRR11 |
| merck-AU121101_a_at | THBS2 LOC1011929523 |
| merck-NM_005990_at | STK10 |
| merck-G36532_at | TIMP3 |
| merck-XM 292021_at | SMCO2 |
| merck-NM_032505_at | KBTBD8 |
| merck-NM_016287_at | HP1BP3 |
| merck-NM_005651_at | TDO2 |
| merck2-AI732388_at | MGAT4A |
| merck2-BC126107_a_at | TEP1 |
| merck2-BX349325_at | PRR11 |
| merck-NM_001747_at | CAPG |
| AFFX-HSAC07/X00351_3_at | ACTB |

### Example 13

### Prognostic Model for Bladder Cancer

This example describes a bladder cancer prognosis model based on gene expression profiling data. The model contains two gene expression signatures as components. In the second part of the example, the number of genes in each signature is reduced to 10 genes to simplify the implementation of this prognosis model.

A total of 273 samples were profiled by Affymetrix® expression arrays. A composite model was built using the first half of samples and the model validated using the second half of samples. In the training set, 137 samples had outcome data (alive or death). In the validation set, 136 had outcome data. The detailed last follow-up dates for the good outcome patients are incomplete. In the training set, 18 out of 47 good outcome patients did not have the last follow-up date. In the validation set, 4 out of 37 good outcome patients did not have the last follow-up date.

A model was built in the training set using a general linear model (from the R package) using the following equation:

Bladder Cancer Risk Score=0.60864−(0.06571*imscore)+(0.06168*hscore) (Formula 27),

where imscore is the immune signature score calculated from signature genes in Table 57 and hscore is the hypoxia signature score calculated from signature genes in Table 58. The scores can be calculated by averaging the log2(intensity) of each probe in the geneset.

The performance of this model is evaluated in reserved validation set of 136 samples. Table 59 lists number of samples, number of deaths, and the death rate in each prediction score bin.

TABLE 59
Average death rate versus prediction score.

| | Number of | Number of | |
|---|---|---|---|
| Score | samples | death | Death Rate |
| <0.6 | 22 | 11 | 0.50 |
| 0.6-0.7 | 38 | 26 | 0.68 |
| 0.7-0.8 | 46 | 37 | 0.80 |
| >0.8 | 30 | 25 | 0.83 |

Using a threshold of 0.66, the odds ratio for overall survival is 4.4 (95% CI: 2.0-9.8), Fisher's Exact Test p-value=3.4×10⁻⁴.

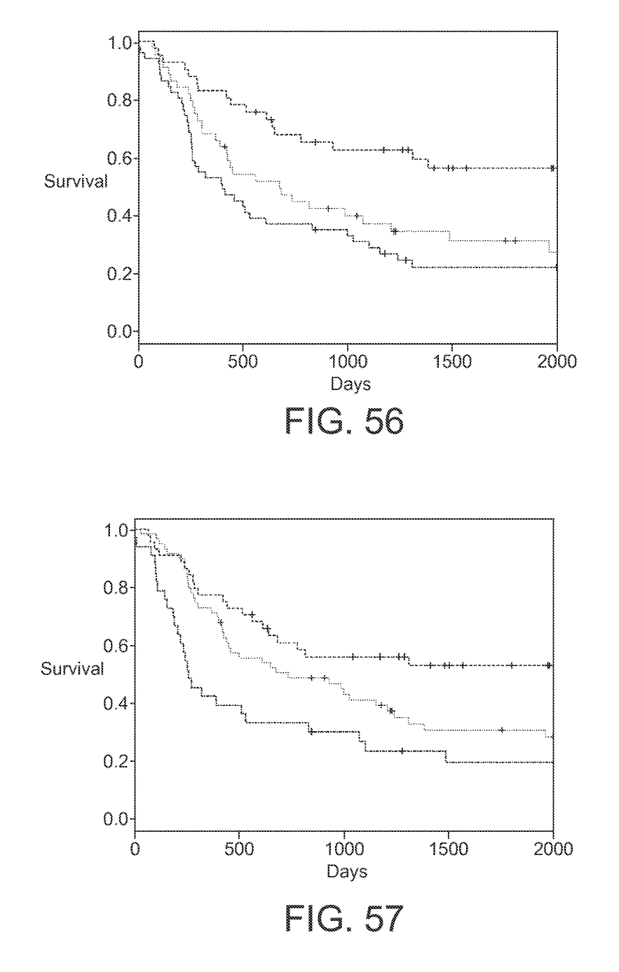

Patients can be further divided into good (risk score<0.66), medium (score 0.66-0.75) and poor (score>0.75) prognosis groups. FIG. 56 shows the Kaplan-Meier curves for these 3 groups. The Chi-square on 2 degrees of freedom is 13.3 (P=1.3×10⁻³).

The number of genes in each pathway can be reduced to 10 genes.

Immune signature:
Probe IDs: merck-NM_002209_at, merck2-BI519527_at, merck-NM_000733_at, merck-NM_001778_at, merck2-NM_052931_at, merck-NM_001767_at, merck-NM_198517_at, merck-NM_024070_at, merck-NM_014207_at, merck-NM_032214_at
Gene symbols: ITGAL, IKZF1, CD3E, CD48, SLAMF6, CD2, TBC1D10C, PVRIG, CD5, SLA2

Hypoxia Signature:
Probe IDs: merck2-NM_005555_at, merck2-X56807_at, merck-BX538327_at, merck-XM_928117_x at, merck2-NM_005554_at, merck-AL572710_s_at, merck-NM_006945_at, merck-X15014_a_at, merck2-AI989728_at, merck-NM_016321_at
Gene symbols: KRT6B, DSC2, DSG3, FAM106B, KRT6A, KRT14, SPRR2D, RALA, SERPINB5, RHCG

The scores derived from these 10-genes are correlated to the original scores at the level of 0.99 for immune signature and 0.89 for the hypoxia signature.

The same model (with the same parameters) was used as Formula 27 for the reduced genesets to estimate the risk score. Table 60 lists number of samples, number of deaths, and the death rate in each prediction score bin.

TABLE 60
Average death rate versus prediction score.

| | Number of | Number of | |
|---|---|---|---|
| Score | samples | death | Death Rate |
| <0.4 | 15 | 7 | 0.47 |
| 0.4-0.6 | 51 | 32 | 0.63 |
| 0.6-0.8 | 50 | 44 | 0.88 |
| >0.8 | 20 | 16 | 0.80 |

Using a threshold of 0.5, the odds ratio for overall survival is 3.7 (95% CI: 1.7-8.1), Fisher's Exact Test p-value=1.7×10.

Patients can be further divided into good (risk score<0.5), medium (score 0.5-0.75) and poor (score>0.75) prognosis groups. FIG. 57 shows the Kaplan-Meier curves for these 3 groups. The Chi-square on 2 degrees of freedom is 12.2 (P=2.2×10⁻³).

TABLE 57
Prognosis signature component 1 (anti-
correlated with poor outcome)

| Probe | Gene |
|---|---|
| merck-NM_005356_at | LCK |
| merck-NM_006144_at | GZMA |
| merck-NM_014207_at | CD5 |
| merck-NM_005608_at | PTPRCAP |
| merck-NM_007181_at | MAP4K1 |
| merck-NM_002738_at | PRKCB |
| merck-Y00638_s_at | PTPRC |
| merck-BC014239_s_at | PTPRC |
| merck-NM_130446_at | KLHL6 |
| merck-NM_005546_at | ITK CYFIP2 |
| merck-NM_006257_at | PRKCQ |
| merck-NM_002104_at | GZMK |
| merck-NM_001504_at | CXCR3 |
| merck-NM_001001895_at | UBASH3A |
| merck-NM_002832_at | PTPN7 |
| merck-NM_018460_at | ARHGAP15 |
| merck-NM_001838_at | CCR7 |
| merck-NM_002209_at | ITGAL |
| merck-NM_006725_at | CD6 |
| merck-BC028068_s_at | JAK3 INSL3 |
| merck-NM_001079_at | ZAP70 |
| merck-NM_005541_at | INPP5D |
| merck-ENST00000318430_s_at | TMC8 |
| merck-NM_006564_at | CXCR6 |
| merck-NM_007237_s_at | SP140 |
| merck-NM_178129_at | P2RY8 |
| merck-NM_000647_s_at | CCR2 |
| merck-BU428565_s_at | P2RY8 |
| merck-NM_002351_s_at | SH2D1A |
| merck-NM_001040033_at | CD53 |
| merck-NM_005816_at | CD96 |
| merck-NM_198517_at | TBC1D10C |
| merck-NM_000733_at | CD3E |
| merck-NM_002163_at | IRF8 |
| merck-NM_000655_at | SELL |
| merck-NM_003037_at | SLAMF1 |
| merck-NM_003151_a_at | STAT4 |
| merck-NM_001007231_s_at | ARHGAP25 |
| merck-NM_018326_at | GIMAP4 |
| merck-NM_000377_at | WAS |
| merck-NM_001558_at | IL10RA |
| merck-NM_002985_at | CCL5 |
| merck-DT807100_at | CD3D CD3G |
| merck-NM_001465_at | FYB |
| merck-BP339517_a_at | FYB |
| merck-NM_030767_at | AKNA |
| merck-NM_005565_at | LCP2 |
| merck-NM_001040031_at | CD37 |
| merck-NM_002872_at | RAC2 |
| merck-NM_019604_at | CRTAM |
| merck-NM_005263_at | GFI1 |
| merck-NM_001037631_at | CTLA4 ICOS |
| merck-NM_016388_at | TRAT1 |
| merck-NM_014450_at | SIT1 RMRP |
| merck-NM_000732_at | CD3D |
| merck-NM_000073_at | CD3G |
| merck-NM_007360_at | KLRK1 KLRC4-KLRK1 |
| merck-NM_013351_at | TBX21 |
| merck-NM_032214_at | SLA2 |
| merck-NM_000639_at | FASLG |
| merck-NM_001242_at | CD27 |
| merck-ENST00000381961_at | IL7R |
| merck-NM_153206_s_at | AMICA1 |
| merck-NM_001025598_at | ARHGAP30 USF1 |
| merck-NM_001768_at | CD8A |
| merck-NM_003978_at | PSTPIP1 |
| merck-NM_014716_at | ACAP1 |
| merck-AK128740_s_at | ILI6 |
| merck-NM_006060_a_at | IKZF1 |
| merck-BC075820_at | IKZF1 |
| merck-NM_016293_at | BIN2 |
| merck-NM_012092_at | ICOS |
| merck-NM_005442_at | EOMES LOC100996624 |
| merck-NM_007074_at | CORO1A |
| merck-NM_000206_at | IL2RG |
| merck-NM_005041_at | PRF1 |
| merck-NM_024898_s_at | DENND1C CRB3 |
| merck-NM_173799_at | TIGIT |
| merck-NM_001767_at | CD2 |
| merck-NM_002348_at | LY9 |
| merck-X60502_s_at | SPN QPRT |
| merck-NM_153236_at | GIMAP7 |
| merck-NM_005601_at | NKG7 |
| merck-NM_032496_at | ARHGAP9 |
| merck-NM_004877_at | GMFG |
| merck-NM_021181_at | SLAMF7 |
| merck-NM_018384_at | GIMAP5 GIMAP1-GIMAP5 |
| merck-NM_181780_at | BTLA |
| merck-NM_001017373_at | SAMD3 |
| merck-NM_000734_at | CD247 |
| merck-NM_003650_at | CST7 |
| merck-NM_172101_at | CD8B |
| merck-NM_001803_at | CD52 |
| merck-NM_001778_at | CD48 |
| merck-NM_001025265_at | CXorf65 |
| merck-NM_198929_at | PYHIN1 |
| merck-ENST00000379833_at | GVINP1 |
| merck-NM_052931_at | SLAMF6 |
| merck-NM_001024667_s_at | FCRL3 |
| merck-NM_002258_at | KLRB1 |
| merck-NM_018556_s_at | SIRPG |
| merck-AK090431_s_at | NLRC3 |
| merck-NM_018990_at | SASH3 XPNPEP2 |
| merck-NM_175900_s_at | C16orf54 QPRT |
| merck-ENST00000316577_s_at | TESPA1 |
| merck-NM_024070_at | PVRIG |
| merck-AY190088_s_at | — |
| merck-NM_001040067_s_at | TRBC2 TRBV3-1 TRBV5-4 |
| | TRBV6-5 TRBV7-2 |
| merck-NM_130848_s_at | C5orf20 |
| merck-ENST00000381153_at | C11orf21 |
| merck-ENST00000382913_s_at | TRAC TRAJ17 TRAV20 |
| | TRDV2 |
| merck-BC030533_s_at | TRBC1 TRBV19 |
| merck-ENST00000244032_a_at | ZNF831 |
| merck-ENST00000371030_at | ZNF831 |
| merck-ENST00000343625_s_at | RASAL3 |
| merck-AF143887_at | — |
| merck-AK128436_at | IKZF3 |
| merck-AI281804_at | GPR174 |
| merck-AF086367_at | — |
| merck-CR598049_at | LINC00426 |
| merck-BM700951_at | KLRK1 KLRC4-KLRK1 |
| merck-BX648371_at | LINC00861 |
| merck-BC070382_at | — |
| merck2-AW798052_at | AKNA |
| merck2-BX640915_at | TIGIT |
| merck2-BM678246_at | CD37 |
| merck2-NM_025228_at | TRAF3IP3 |
| merck2-XM_033379_at | WDFY4 |
| merck2-AJ515553_at | AMICA1 |
| merck2-BP262340_at | IL16 |
| merck2-AK225623_at | DENND1C CRB3 |
| merck2-AL833681_at | CD96 |
| merck2-BF111803_at | ARHGAP15 |
| merck2-BX406128_at | CD3G |
| merck2-NM_153701_at | — |
| merck2-BC020657_at | GIMAP4 |
| merck2-AY185344_at | PYHIN1 |
| merck2-DR159064_at | EOMES LOC100996624 |
| merck2-ENST00000390420_at | TRBV3-1 TRBV5-4 |
| | TRBV6-5 TRBV7-2 |
| merck2-ENST00000390420_s_at | — |
| merck2-NM_001010923_at | THEMIS |
| merck2-ENST00000390409_at | TRBC1 TRBV19 |
| merck2-AX721088_at | — |
| merck2-ENST00000390393_at | TRBV19 |
| merck2-AW341086_at | — |
| merck2-AA278761_at | — |
| merck2-AA278761_x_at | — |
| merck2-ENST00000390394_s_at | — |
| merck2-AA669142_at | — |
| merck2-AW007991_at | PTPRC |
| merck2-BG743900_at | PRKCB |
| merck2-X06318_at | PRKCB |
| merck2-BI519527_at | IKZF1 |
| merck2-ENST00000390537_s_at | — |
| merck2-AY292266_x_at | — |
| merck2-NM_005816_a_at | CD96 |
| merck2-NM_198196_a_at | CD96 |
| merck2-NM_001114380_x_at | ITGAL |
| merck2-NM_007237_a_at | SP140 |
| merck2-NM_007237_at | SP140 |
| merck2-NM_052931_at | SLAMF6 |
| merck2-NM_001558_at | IL10RA |
| merck2-NM_007360_at | KLRK1 KLRC4-KLRK1 |
| merck2-NM_002209_x_at | ITGAL |
| merck2-NM_175900_at | C16orf54 QPRT |

TABLE 58
Prognosis signature component 2 (correlated with poor outcome)

| probe | Gene |
|---|---|
| merck-NM_002627_at | PFKP PITRM1 |
| merck-NM_000302_at | PLOD1 |
| merck-NM_001216_at | CA9 RMRP |
| merck-ENST00000377093_at | KIF1B |
| merck-BC004202_a_at | CHEK1 |
| merck-NM_030949_at | PPP1R14C |
| merck-CR593119_a_at | CLIC4 |
| merck-NM_001255_s_at | CDC20 |
| merck-BG679113_s_at | KRT6A KRT6B KRT6C |
| merck-NM_002421_at | MMP1 |
| merck-BQ217236_a_at | SERPINB5 |
| merck-NM_001793_at | CDH3 |
| merck-NM_001238_at | CCNE1 |
| merck-BU597348_s_at | SYNCRIP |
| merck-NM_006516_at | SLC2A1 |
| merck-BX648425_a_at | DSC2 |
| merck-X15014_a_at | RALA |
| merck-NM_018685_at | ANLN |
| merck-CR614206_a_at | ERO1L |
| merck-NM_001124_at | ADM |
| merck-NM_015440_at | MTHFD1L |
| merck-ENST00000367307_a_at | MTHFD1L |
| merck-NM_058179_at | PSAT1 |
| merck-NM_031415_s_at | GSDMC |
| merck-NM_005557_x_at | KRT16 |
| merck-NM_053016_at | PALM2 PALM2-AKAP2 |
| merck-CR602579_a_at | CTPS1 |
| merck-NM_001428_s_at | ENO1 |
| merck-ENST00000305850_at | CENPN CMC2 |
| merck-NM_005978_at | S100A2 |
| merck-NM_018643_at | TREM1 |
| merck-NM_006505_at | PVR |
| merck-NM_080655_s_at | MSANTD3 |
| merck-NM_001012507_at | CENPW |
| merck-ENST00000258005_a_at | NHSL1 |
| merck-AK129763_at | LINC00673 |
| merck-XM_927868_s_at | PGK1 |
| merck-XM_928117_x_at | FAM106B |
| merck-AL359337_at | ADM |
| merck-AA148856_s_at | SYNCRIP |
| merck2-AI989728_at | SERPINB5 |
| merck2-DQ892208_at | CA9 RMRP |
| merck2-AK022036_at | WWTR1 |
| merck2-AA677426_at | — |
| merck2-AA677426_s_at | — |
| merck2-BC004856_at | NCS1 |
| merck2-BG252150_at | PFKP |
| merck2-BC007633_at | AGO2 |
| merck2-BG400371_at | — |
| merck2-DQ891441_at | — |
| merck2-NM_017522_AS_at | LRP8 |
| merck2-AF039652_at | RNASEH1 |
| merck2-AV714642_at | ANLN |
| merck2-AB030656_at | CORO1C |
| merck2-NM_000291_at | PGK1 |
| merck2-NM_005554_at | KRT6A |
| merck2-BC002829_at | S100A2 |
| merck2-BU681245_at | — |
| merck2-AK225899_a_at | CTPS1 |
| merck2-BC062635_a_at | XPO5 |
| merck2-AF257659_a_at | CALU |
| merck2-CA308717_at | — |
| merck2-X56807_at | DSC2 |
| merck2-CR936650_at | ANLN |
| merck2-AY423725_a_at | PGK1 |
| merck2-BC103752_a_at | PGK1 |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for predicting prognosis of a patient with breast cancer, comprising:
(a) determining from a tumor biopsy sample from the subject gene expression intensities for each of the following categories of signature genes:
(1) estrogen receptor (ER),
(2) human epidermal growth factor receptor 2 (HER2),
(3) at least 5 proliferation signature genes listed in Table 1, and
(4) at least 5 immune signature genes listed in Table 2; and
(b) calculating a breast cancer risk score from the gene expression intensities;
wherein a high breast cancer risk score is an indication that the subject has a high risk for bone metastasis and death.

2. The method of claim 1, wherein the at least 5 proliferation signature genes are selected from the group consisting of TPX2, CENPA, KIF2C, CCNB2, BUB1, HJURP, CDCA5, CEP55, and SKA1

3. The method of claim 1, wherein the at least 5 immune signature genes are selected from the group consisting of CD3D, CD2, CD3E, ITK, TRBC1, TBC1D10C, ACAP1, CD247, SLAMF6, and IKZF1.

4. The method of claim 1, further comprising treating the subject with more aggressive treatment if the subject has a high breast cancer risk score.

5. A method for predicting prognosis of a patient with lung cancer, comprising:
(a) determining from a tumor biopsy sample from the subject gene expression intensities for each of the following categories of signature genes:
(1) at least 5 immune signature genes listed in Table 4,
(2) at least 5 hypoxia signature genes listed in Table 5,
(3) at least 5 lung cancer prognosis signature genes listed in Table 7, and
(4) at least 5 proliferation signature genes listed in Table 8;
(b) determining the composite tumor stage; and
(c) calculating a lung cancer risk score from the gene expression intensities and composite tumor stage;
wherein a high lung cancer risk score is an indication that the subject has a high risk of death.

6. The method of claim 5, wherein the at least 5 immune signature genes are selected from the group consisting of CD2, ITGAL, IKZF1, CD3D, TRBC1, ACAP1, CD3E, TBC1D10C, CD247, and SLAMF6.

7. The method of claim 5, wherein the at least 5 hypoxia signature genes are selected from the group consisting of SLC2A1, S100A2, KRT16, KRT6A, CD109, GJB3, SFN, MICALL1, RNTL2, and COL7A1.

8. The method of claim 5, wherein the at least 5 lung cancer prognosis signature genes are selected from the group consisting of HLF, SCN7A, NR3C2, PCDP1, ABCA8, EMCN, IFT57, BDH2, MAMDC2, and ITGA8.

9. The method of claim 5, wherein the at least 5 proliferation signature genes are selected from the group consisting of TPX2, CENPA, KIF2C, CCNB2, CDCA5, HJURP, KIF4A, BIRC5, DLGAP5, and SKA1.

10. The method of claim 5 further comprising treating the subject with more aggressive treatment if the subject has a high lung cancer risk score.

11. A method for predicting prognosis of a patient with colon cancer, comprising:
(a) determining from a tumor biopsy sample from the subject gene expression intensities for each of the following categories of signature genes:
(1) at least 5 immune signature genes listed in Table 12,
(2) at least 5 hypoxia signature genes listed in Table 13,
(3) at least 5 vimentin (VIM) correlated genes listed in Table 14,
(4) at least 5 CDH1 correlated genes listed in Table 15,
(5) at least 5 first prognosis signature genes listed in Table 16, and
(6) at least 5 second prognosis signature genes listed in Table 17;
(b) determining the composite tumor stage; and
(c) calculating a colon cancer risk score from the gene expression intensities and composite tumor stage; wherein a high colon cancer risk score is an indication that the subject has a high risk of death.

12. The method of claim 11, wherein the at least 5 immune signature genes are selected from the group consisting of IKZF1, ITGAL, CD2, ITK, MAP4K1, CD3E, TBC1D10C, TRBC2, CD247, and CD3D.

13. The method of claim 11, wherein the at least 5 hypoxia signature genes are selected from the group consisting of SLC2A1, RALA, ERO1L, ANLN, S100A2, PHLDA2, CDC20, LAMC2, PLAUR, and SLC16A3.

14. The method of claim 11, wherein the at least 5 vimentin (VIM) correlated genes are selected from the group consisting of CCDC80, VIM, HEG1, CNRIP1, RAB31, EFEMP2, GNB4, MRAS, CMTM3, and TIMP2.

15. The method of claim 11, wherein the at least 5 CDH1 correlated genes are selected from the group consisting of ELF3, CLDN7, CLDN4, CDH1, RAB25, ESRP1, ESRP2, ERBB3, AP1M2, and EPCAM.

16. The method of claim 11, wherein the at least 5 first prognosis signature genes are selected from the group consisting of MZB1, OR6C4 IGKV3-11 IGKV3D-11 IGKV3D-20 RHNO1, TNFRSF17, IGKC IGKV1D-39 IGKV1-39, IGHA1 IGHG1 IGH, IGLC1, IGKC IGKV1-16 IGKV1D-16, IGLV6-57, IGLV1-40 IGLV5-39, and IGJ.

17. The method of claim 11, wherein the at least 5 second prognosis signature genes are selected from the group consisting of SPP1, CDH2, ITGB1, SERPINE1, PLOD2, COL4A1, NTM, MPRIP, PLIN2, and TIMP1.

18. The method of claim 11, further comprising treating the subject with more aggressive treatment if the subject has a high colon cancer risk score.

19. A method for predicting prognosis of a patient with kidney cancer, comprising:
(a) determining from a tumor biopsy sample from the subject gene expression intensities for each of the following categories of signature genes:
(1) at least 5 first prognosis signature genes listed in Table 22, and
(2) at least 5 second prognosis signature genes listed in Table 23; and
(b) calculating a kidney cancer risk score from the gene expression intensities; wherein a high kidney cancer risk score is an indication that the subject has a high risk of death.

20. The method of claim 19, wherein the at least 5 first prognosis signature genes are selected from the group consisting of CRY2, NR3C2, HLF, EMX2OS, FAM221B, BDH2, BCL2, ACADL, NDRG2, and NPR3.

21. The method of claim 19, wherein the at least 5 second prognosis signature genes are selected from the group consisting of TPX2, CCNB2, AURKB, HJURP, CENPA, CENPF, SKA1, CEP55, PTTG1, and FOXM1

22. The method of claim 19, further comprising treating the subject with more aggressive treatment if the subject has a high kidney cancer risk score.

23-70. (canceled)
